SEQUENCE LISTING (2 sequences)

SEQ ID NO: 1 - protein, 449 aa, Artificial Sequence
  Cetuximab heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  Cetuximab light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

(19) United States
(12) Patent Application Publication
(10) Pub. No.: US 2022/0288208 A1
(43) Pub. Date: Sep. 15, 2022

(54) CETUXIMAB-IR700 CONJUGATE COMPOSITIONS

(71) Applicant: Rakuten Medical, Inc., San Diego, CA (US)

(72) Inventors: Anthony MANIBUSAN, San Diego, CA (US); Lewis R. MAKINGS, Encinitas, CA (US)

(73) Assignee: Rakuten Medical, Inc., San Diego, CA (US)

(21) Appl. No.: 17/632,756

(22) PCT Filed: Aug. 6, 2020
(86) PCT No.: PCT/US2020/045283

§ 371 Date: Feb. 3, 2022

Related U.S. Application Data
(60) Provisional application No. 62/883,825, filed on Aug. 7, 2019.

(51) Int. Cl. A61K 41/00; C07K 16/28; A61N 5/06

(52) U.S. Cl. CPC A61K 41/0057; C07K 16/2863; A61N 5/062; A61N 2005/0663; A61N 2005/0659

(57) ABSTRACT

Provided are conjugates of the phthalocyanine dye IR700 and an EGFR-binding antibody, such as a cetuximab antibody, and pharmaceutical compositions thereof. In some aspects, the compositions contain an EGFR-binding antibody, such as a cetuximab antibody, that is modified by conjugation to the IR700 dye at specific positions within the heavy chain and/or the light chain of the cetuximab antibody. In some aspects, such conjugates are capable of targeted cell killing following irradiation of the conjugate Also provided are related methods of manufacture and methods of use and uses, including in treatments for tumors and specific-cancer indications.

### CROSS-REFERENCE TO RELATED APPLICATIONS

This application claims priority from U.S. Provisional Application No. 62/883,825, filed Aug. 7, 2019, entitled “CETUXIMAB-IR700 CONJUGATE COMPOSITIONS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 751702001640SeqList.txt, created Aug. 4, 2020, which is 5.84 kilobytes in size. The information in the electronic format of the Sequence Listing is incorporated by reference in its entirety.

### FIELD

The present disclosure relates to conjugates of the phthalocyanine dye IR700 and an EGFR-binding antibody, such as a cetuximab antibody, and pharmaceutical compositions thereof. In some aspects, the compositions contain an EGFR-binding antibody, such as a cetuximab antibody, that is modified by conjugation to the IR700 dye at specific positions within the heavy chain and/or the light chain of the cetuximab antibody. In some aspects, such conjugates are capable of targeted cell killing following irradiation of the conjugate. Also provided are related methods of manufacture and methods of use and uses, including in treatments for tumors and specific-cancer indications.

### BACKGROUND

Various therapies are available for treating disease, such as cancer. For example, photoimmunotherapy (PIT) is a method that uses a photosensitizer conjugated to an antibody or other targeting molecule to target to a cell surface target molecule, e.g., a cell surface receptor, in order to permit the targeted killing of specific cells. In some cases, PIT can selectively target disease cells, such as tumor cells, and thereby selectively kill such cells without damaging healthy cells. Improved strategies are needed to improve phthalocyanine dye conjugates for use in PIT. Provided are compositions and methods that meet such needs.

### SUMMARY

Provided herein are conjugates, such as an antibody-phthalocyanine dye conjugate, for example, a cetuximab-IR700 conjugate. In some of any of the provided embodiments, the conjugate includes at least two molecules of IR700 conjugated to at least two lysine (K) positions in a cetuximab, and wherein the at least two lysine positions are independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab.

In some of any embodiments, the conjugate includes at least three molecules of IR700 conjugated to at least three lysine positions in the cetuximab. In some of any embodiments, the at least three lysine positions are independently selected from the group consisting of K107, K145, K188, K190, and K207 in the light chain and K5, K75, K215, K248, K292, K328, K336, K416, and K449 in the heavy chain. In some of any embodiments, at least one of the lysine positions conjugated to IR700 is selected from the group consisting of K145 in the light chain or K215, K416 or K449 in the heavy chain. In some of any embodiments, at least one molecule of IR700 is conjugated to a lysine in the light chain, and at least one molecule of IR700 is conjugated to a lysine in the heavy chain.

In some of any embodiments, the conjugate is capable of being activated by light irradiation with a wavelength between 690 nm±50 nm and thereby exhibits a cell killing activity when then conjugate is bound to an epitope on the surface of the cell.

Also provided herein are compositions. In some of any of the provided embodiments, the compositions contain any of the conjugates described herein and a pharmaceutically acceptable excipient.

Also provided herein are compositions that include a population of conjugates, wherein the conjugates in the population comprise IR700 conjugated to a cetuximab, wherein at least at or about 50%, 60%, 70%, 80%, 90%, or more than at or about 90% of the conjugates comprise at least two molecules of IR700 conjugated to at least two lysine (K) positions in the cetuximab, and wherein the two lysine positions are independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab.

In some of any embodiments, at least at or about 50%, 60%, 70%, 80%, 90%, or more than at or about 90% of the conjugates comprise at least three molecules of IR700 conjugated to at least three lysine positions in the cetuximab. In some of any embodiments, at least three lysine positions are independently selected from the group consisting of K107, K145, K188, K190, and K207 in the light chain and K5, K75, K215, K248, K292, K328, K336, K416, and K449 in the heavy chain.

In some of any embodiments, at least at or about 50%, 60%, 70%, 80%, 90%, or more than at or about 90% of the conjugates comprise at least one molecule of IR700 conjugated to K145 in the light chain or K215, K416 or K449 in the heavy chain. In some of any embodiments, at least at or about 50%, 60%, 70%, 80%, 90%, or more than at or about 90% of the conjugates comprise a molecule of IR700 conjugated to K145 in the light chain and a molecule of IR700 conjugated to at least one of K215, K416 or K449 in the heavy chain.

In some of any embodiments, the ratio of IR700 molecule to the cetuximab is between about 2:1 to about 4:1. In some of any embodiments, the ratio of IR700 molecule to the cetuximab is about 2.5:1, 2.6:1, 2.7:1, 2.8:1, 2.9:1, 3.0:1, 3.1:1, 3.2:1, 3.3:1 or 3.4:1. In some of any embodiments, the ratio of IR700 molecule to the cetuximab is between about 2.7:1 to about 3.2:1.

In some of any embodiments, no more than at or about 15% of the cetuximab molecules in the composition are unconjugated with IR700. In some of any embodiments, less than at or about 10% of the cetuximab molecules in the composition are unconjugated with IR700.

In some of any embodiments, the percentage of free dye in the composition is less than at or about 3%, less than at or about 2%, less than at or about 1%, or less than at or about 0.5%.

Also provided herein are compositions that include a population of cetuximab-IR700 conjugates, wherein a plurality of the conjugates in the composition each comprise IR700 conjugated to a cetuximab, at a lysine (K) in the light chain or the heavy chain of the cetuximab selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (1(215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab, and the composition comprises the features of: (a) the ratio of IR700 molecules in the composition to the cetuximab molecules in the population is between about 2:1 and about 3:5, (b) less than at or about 10% of the cetuximab molecules are unconjugated with IR700, and (c) the percentage of free dye in the composition among all dye molecules in the composition is less than at or about 3%.

In some of any embodiments, a plurality of the conjugates comprise a cetuximab conjugated with IR700 at K145 of the light chain. In some of any embodiments, a plurality of the conjugates comprise a cetuximab conjugated with IR700 at K215, K416 or K449 of the heavy chain. In some of any embodiments, a plurality of the conjugates comprise a cetuximab conjugated with at least three molecules of IR700.

In some of any embodiments, the plurality comprises at least at or about 51%, at least at or about 55%, at least at or about 60%, at least at or about 70%, at least at or about 75% or at least at or about 80% of the conjugates in the composition.

In some of any embodiments, the percentage of free dye in the composition is less than at or about 2%, less than at or about 1%, or less than at or about 0.5%.

In some of any embodiments, the cetuximab comprises a heavy chain sequence set forth in SEQ ID NO: 1, a light chain sequence set forth in SEQ ID NO:2, or a combination thereof.

In some of any embodiments, the percentage of free dye in the composition is substantially unchanged after storage for 6 months in dark or reduced light conditions. In some of any embodiments, the composition comprises at least at or about 95%, 96% 97% or 98% monomeric form of the conjugate. In some of any embodiments, the composition comprises less than at or about 5%, 4% or 3% high molecular weight species.

Also provided are methods related to any of the conjugates or compositions provided herein. In some of any of the embodiments, provided methods include methods of killing a tumor or a cancer cell. In some of any of the provided embodiments, the methods involve administering a pharmaceutical composition comprising any of the conjugates or the compositions described herein, to a site at or proximal to the tumor or the cancer cell; and irradiating an area proximal to the tumor or the cancer cell at a wavelength of about 600 nm to about 850 nm at a dose of from about 25 J cm⁻² to about 400 J cm⁻² or from about 25 J/cm of fiber length to about 500 J/cm of fiber length, thereby killing the tumor or the cancer cell.

Also provided are methods of treating a disease or condition in a subject. In some of any of the embodiments, the methods involve administering a pharmaceutical composition comprising any of the conjugates or the compositions described herein to a site at or proximal to the tumor or the cancer cell; and irradiating an area proximal to a lesion or tumor in the subject at a wavelength of about 600 nm to about 850 nm at a dose of from about 25 J cm⁻² to about 400 J cm⁻² or from about 25 J/cm of fiber length to about 500 J/cm of fiber length, thereby treating the disease or condition.

In some of any embodiments, the irradiating step is carried out at a wavelength of 690±50 nm or at a wavelength of or about 690±20 nm. In some of any embodiments, the irradiating step is carried out at a wavelength of about 690 nm.

In some of any embodiments, the disease or condition is a tumor or cancer. In some of any embodiments, the tumor or the cancer cell or the disease or condition is a tumor that is a carcinoma of the bladder, pancreas, colon, ovary, lung, breast, stomach, prostate, cervix, esophagus or head and neck. In some of any embodiments, the tumor or the cancer cell or the disease or condition is a cancer that is located at the head and neck, breast, liver, colon, ovary, prostate, pancreas, brain, cervix, bone, skin, eye, bladder, stomach, esophagus, peritoneum, or lung. In some of any embodiments, the cancer is a head and neck cancer.

Also provided are methods of manufacturing a conjugate, such as a cetuximab-IR700 conjugate described herein. In some of any of the embodiments, provided are methods of manufacturing a stable conjugate. In some of any of the embodiments, the methods involve a) contacting a cetuximab with an IR700 under conditions to produce a cetuximab-IR700 conjugate, wherein the conjugate comprises at least two lysine (K) positions conjugated to IR700 independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab; b) subjecting the conjugate to a step during and/or subsequent to conjugation which substantially reduces IR700 non-specifically associated with the cetuximab; c) formulating the conjugate in a pharmaceutically acceptable buffer, wherein in each of steps a)-c), the only light to which the dye and conjugate are exposed has a wavelength within a range of about 400 nm to about 650 nm or has an intensity of less than at or about 500 lux.

In some of any embodiments, step b) comprises subjecting the conjugate to a glycine quenching reaction after completion of the conjugation reaction between IR700 and the cetuximab. In some of any embodiments, the quenching reaction is performed overnight or for a duration of greater than at or about 6 hours.

Also provided are table conjugates. In some of any of the embodiments, the stable conjugates are manufactured by any of the methods of manufacturing conjugates described herein.

Also provided herein are compositions that include a plurality of conjugates, wherein the conjugates comprise IR700 conjugated to a cetuximab, wherein trypsin digestion of the composition produces a population of peptides comprising: a) peptides of the heavy chain of cetuximab comprising an IR700 molecule conjugated to the lysine corresponding to position 215 (K215) of SEQ ID NO: 1; b) peptides of the heavy chain of cetuximab comprising an IR700 molecule conjugated to the lysine corresponding to position 292 (K292) of SEQ ID NO: 1; c) peptides of the heavy chain of cetuximab comprising an IR700 molecule conjugated to the lysine corresponding to position 416 (K416) of SEQ ID NO: 1; and d) peptides of the light chain of cetuximab comprising an IR700 molecule conjugated to the lysine corresponding to position 145 (K145) of SEQ ID NO: 2.

In some of any embodiments, the population of peptides further comprises: e) peptides of the heavy chain of cetuximab comprising an IR700 molecule conjugated to the lysine corresponding to position 336 (K336) of SEQ ID NO: 1; and f) peptides of the heavy chain of cetuximab comprising an IR700 molecule conjugated to the lysine corresponding to position 449 (K449) of SEQ ID NO: 1.

In some of any embodiments, the population of peptides further comprises: g) peptides of the light chain of cetuximab comprising an IR700 molecule conjugated to the lysine corresponding to position 107 (K107) of SEQ ID NO: 2; h) peptides of the light chain of cetuximab comprising an IR700 molecule conjugated to the lysine corresponding to position 190 (K190) of SEQ ID NO: 2; i) peptides of the heavy chain of cetuximab comprising an IR700 molecule conjugated to the lysine corresponding to position 5 (K5) of SEQ ID NO: 1; and j) peptides of the heavy chain of cetuximab comprising an IR700 molecule conjugated to the lysine corresponding to position 75 (K75) of SEQ ID NO: 1.

In some of any embodiments, the population of peptides further comprises one or more of: k) peptides of the heavy chain of cetuximab comprising an IR700 molecule conjugated to the lysine corresponding to position 248 (K248) of SEQ ID NO: 1; 1) peptides of the heavy chain of cetuximab comprising an IR700 molecule conjugated to the lysine corresponding to position 328 (K328) of SEQ ID NO: 1; m) peptides of the light chain of cetuximab comprising an IR700 molecule conjugated to the lysine corresponding to position 188 (K188) of SEQ ID NO: 2; and n) peptides of the light chain of cetuximab comprising an IR700 molecule conjugated to the lysine corresponding to position 207 (K207) of SEQ ID NO: 2.

Also provided are compositions that include a plurality of conjugates, wherein the conjugates comprise IR700 conjugated to a cetuximab, wherein trypsin digestion of the composition produces a population of peptides comprising: a) peptides of the heavy chain of cetuximab comprising an IR700 molecule conjugated to the lysine corresponding to position 5 (K5) of SEQ ID NO: 1; b) peptides of the heavy chain of cetuximab comprising an IR700 molecule conjugated to the lysine corresponding to position 75 (K75) of SEQ ID NO: 1; c) peptides of the heavy chain of cetuximab comprising an IR700 molecule conjugated to the lysine corresponding to position 215 (1(215) of SEQ ID NO: 1; d) peptides of the heavy chain of cetuximab comprising an IR700 molecule conjugated to the lysine corresponding to position 248 (K248) of SEQ ID NO: 1; e) peptides of the heavy chain of cetuximab comprising an IR700 molecule conjugated to the lysine corresponding to position 292 (K292) of SEQ ID NO: 1; f) peptides of the heavy chain of cetuximab comprising an IR700 molecule conjugated to the lysine corresponding to position 328 (K328) of SEQ ID NO: 1; g) peptides of the heavy chain of cetuximab comprising an IR700 molecule conjugated to the lysine corresponding to position 336 (K336) of SEQ ID NO: 1; h) peptides of the heavy chain of cetuximab comprising an IR700 molecule conjugated to the lysine corresponding to position 416 (K416) of SEQ ID NO: 1; i) peptides of the heavy chain of cetuximab comprising an IR700 molecule conjugated to the lysine corresponding to position 449 (K449) of SEQ ID NO: 1; j) peptides of the light chain of cetuximab comprising an IR700 molecule conjugated to the lysine corresponding to position 107 (K107) of SEQ ID NO: 2; k) peptides of the light chain of cetuximab comprising an IR700 molecule conjugated to the lysine corresponding to position 145 (K145) of SEQ ID NO: 2; l) peptides of the light chain of cetuximab comprising an IR700 molecule conjugated to the lysine corresponding to position 188 (K188) of SEQ ID NO: 2; m) peptides of the light chain of cetuximab comprising an IR700 molecule conjugated to the lysine corresponding to position 190 (K190) of SEQ ID NO: 2; and n) peptides of the light chain of cetuximab comprising an IR700 molecule conjugated to the lysine corresponding to position 207 (1(207) of SEQ ID NO: 2.

In some of any embodiments, the peptides are detected by positive ion mode mass spectrometry. In some of any embodiments, when extracted ion chromatograms (EIC) are generated for the peptides detected by the positive ion mode mass spectrometry: the integrated area of the EIC peaks corresponding to peptides of a) is between at or about 3% and at or about 5% of the sum of the integrated area of the EIC peaks of the corresponding unmodified peptides and the integrated area of the EIC peaks corresponding the peptides of a); the integrated area of the EIC peaks corresponding to peptides of b) is between at or about 3% and at or about 5% of the sum of the integrated area of the EIC peaks of the corresponding unmodified peptide and the integrated area of the EIC peaks corresponding the peptides of b); the integrated area of the EIC peaks corresponding to peptides of c) is between at or about 8% and at or about 11% of the sum of the integrated area of the EIC peaks of the corresponding unmodified peptide and the integrated area of the EIC peaks corresponding the peptides of c); the integrated area of the EIC peaks corresponding to peptides of d) is between at or about 0.5% and at or about 2.5% of the sum of the integrated area of the EIC peaks of the corresponding unmodified peptide and the integrated area of the EIC peaks corresponding the peptides of d); the integrated area of the EIC peaks corresponding to peptides of e) is between at or about 8% and at or about 12% of the sum of the integrated area of the EIC peaks of the corresponding unmodified peptide and the integrated area of the EIC peaks corresponding the peptides of e); the integrated area of the EIC peaks corresponding to peptides of f) is between at or about 0.2% and at or about 2.5% of the sum of the integrated area of the EIC peaks of the corresponding unmodified peptide and the integrated area of the EIC peaks corresponding the peptides of f); the integrated area of the EIC peaks corresponding to peptides of g) is between at or about 4.5% and at or about 7% of the sum of the integrated area of the EIC peaks of the corresponding unmodified peptide and the integrated area of the EIC peaks corresponding the peptides of g); the integrated area of the EIC peaks corresponding to peptides of h) is between at or about 9.5% and at or about 13% of the sum of the integrated area of the EIC peaks of the corresponding unmodified peptide and the integrated area of the EIC peaks corresponding the peptides of h); the integrated area of the EIC peaks corresponding to peptides of i) is between at or about 6% and at or about 10% of the sum of the integrated area of the EIC peaks of the corresponding unmodified peptide and the integrated area of the EIC peaks corresponding the peptides of i); the integrated area of the EIC peaks corresponding to peptides of j) is between at or about 2% and at or about 5% of the sum of the integrated area of the EIC peaks of the corresponding unmodified peptide and the integrated area of the EIC peaks corresponding the peptides of j); the integrated area of the EIC peaks corresponding to peptides of k) is between at or about 7% and at or about 11% of the sum of the integrated area of the EIC peaks of the corresponding unmodified peptide and the integrated area of the EIC peaks corresponding the peptides of k); the integrated area of the EIC peaks corresponding to peptides of l) is between at or about 0.5% and at or about 4% of the sum of the integrated area of the EIC peaks of the corresponding unmodified peptide and the integrated area of the EIC peaks corresponding the peptides of l); the integrated area of the EIC peaks corresponding to peptides of m) is between at or about 1.5% and at or about 5% of the sum of the integrated area of the EIC peaks of the corresponding unmodified peptide and the integrated area of the EIC peaks corresponding the peptides of m); and the integrated area of the EIC peaks corresponding to peptides of n) is between at or about 0.5% and at or about 4% of the sum of the integrated area of the EIC peaks of the corresponding unmodified peptide and the integrated area of the EIC peaks corresponding the peptides of n).

In some of any embodiments, the integrated area of the EIC peaks corresponding to peptides of a) is about 3.8±1% of the sum of the integrated area of the EIC peaks of the corresponding unmodified peptides and the integrated area of the EIC peaks corresponding the peptides of a); the integrated area of the EIC peaks corresponding to peptides of b) is about 3.5±1% of the sum of the integrated area of the EIC peaks of the corresponding unmodified peptides and the integrated area of the EIC peaks corresponding the peptides of b); the integrated area of the EIC peaks corresponding to peptides of c) is about 10.0±1% of the sum of the integrated area of the EIC peaks of the corresponding unmodified peptides and the integrated area of the EIC peaks corresponding the peptides of c); the integrated area of the EIC peaks corresponding to peptides of d) is about 1.7±1% of the sum of the integrated area of the EIC peaks of the corresponding unmodified peptides and the integrated area of the EIC peaks corresponding the peptides of d); the integrated area of the EIC peaks corresponding to peptides of e) is about 10.2±1% of the sum of the integrated area of the EIC peaks of the corresponding unmodified peptides and the integrated area of the EIC peaks corresponding the peptides of e); the integrated area of the EIC peaks corresponding to peptides of f) is about 1.3±1% of the sum of the integrated area of the EIC peaks of the corresponding unmodified peptides and the integrated area of the EIC peaks corresponding the peptides of f); the integrated area of the EIC peaks corresponding to peptides of g) is about 5.9±1% of the sum of the integrated area of the EIC peaks of the corresponding unmodified peptides and the integrated area of the EIC peaks corresponding the peptides of g); the integrated area of the EIC peaks corresponding to peptides of h) is about 11.2±1% of the sum of the integrated area of the EIC peaks of the corresponding unmodified peptides and the integrated area of the EIC peaks corresponding the peptides of h); the integrated area of the EIC peaks corresponding to peptides of i) is about 7.6±1% of the sum of the integrated area of the EIC peaks of the corresponding unmodified peptides and the integrated area of the EIC peaks corresponding the peptides of i); the integrated area of the EIC peaks corresponding to peptides of j) is about 3.4±1% of the sum of the integrated area of the EIC peaks of the corresponding unmodified peptides and the integrated area of the EIC peaks corresponding the peptides of j); the integrated area of the EIC peaks corresponding to peptides of k) is about 9.3±1% of the sum of the integrated area of the EIC peaks of the corresponding unmodified peptides and the integrated area of the EIC peaks corresponding the peptides of k); the integrated area of the EIC peaks corresponding to peptides of l) is about 2.1±1% of the sum of the integrated area of the EIC peaks of the corresponding unmodified peptides and the integrated area of the EIC peaks corresponding the peptides of l); the integrated area of the EIC peaks corresponding to peptides of m) is about 3.5±1% of the sum of the integrated area of the EIC peaks of the corresponding unmodified peptides and the integrated area of the EIC peaks corresponding the peptides of m); and the integrated area of the EIC peaks corresponding to peptides of n) is about 2.0±1% of the sum of the integrated area of the EIC peaks of the corresponding unmodified peptides and the integrated area of the EIC peaks corresponding the peptides of n).

In some of any of the provided embodiments, the amino acid sequence of the peptides of a) corresponds to amino acids 1-38 of SEQ ID NO: 1; the amino acid sequence of the peptides of b) corresponds to amino acids 72-81 of SEQ ID NO: 1; the amino acid sequence of the peptides of c) corresponds to amino acids 213-216 of SEQ ID NO: 1; the amino acid sequence of the peptides of d) corresponds to amino acids 225-250 of SEQ ID NO: 1; the amino acid sequence of the peptides of e) corresponds to amino acids 291-294 of SEQ ID NO: 1; the amino acid sequence of the peptides of f) corresponds to amino acids 325-336 of SEQ ID NO: 1; the amino acid sequence of the peptides of g) corresponds to amino acids 329-340 of SEQ ID NO: 1; the amino acid sequence of the peptides of h) corresponds to amino acids 412-418 of SEQ ID NO: 1; the amino acid sequence of the peptides of i) corresponds to amino acids 442-449 of SEQ ID NO: 1; the amino acid sequence of the peptides of j) corresponds to amino acids 104-108 of SEQ ID NO: 2; the amino acid sequence of the peptides of k) corresponds to amino acids 143-149 of SEQ ID NO: 2; the amino acid sequence of the peptides of l) corresponds to amino acids 184-190 of SEQ ID NO: 2; the amino acid sequence of the peptides of m) corresponds to amino acids 189-207 of SEQ ID NO: 2; and the amino acid sequence of the peptides of n) corresponds to amino acids 191-211 of SEQ ID NO: 2.

Also provided herein are compositions that include a plurality of conjugates, wherein the conjugates comprise IR700 conjugated to cetuximab, and wherein trypsin digestion of the composition produces peptides that generate mass spectra comprising extracted ion chromatogram (EIC) peaks corresponding to: peptides comprising an IR700 molecule conjugated to a lysine corresponding to position 215 (K215) of SEQ ID NO: 1, wherein the percent area of the conjugated EIC peak is at least at or about 9% of the total area of EIC peaks of the corresponding modified and unmodified polypeptide; peptides comprising an IR700 molecule conjugated to a lysine corresponding to position 292 (K292) of SEQ ID NO: 1, wherein the percent area of the conjugated EIC peak is at least at or about 8% of the total area of EIC peaks of the corresponding modified and unmodified polypeptide; peptides comprising an IR700 molecule conjugated to a lysine corresponding to position 416 (K416) of SEQ ID NO: 1, wherein the percent area of the conjugated EIC peak is at least at or about 8% of the total area of EIC peaks of the corresponding modified and unmodified polypeptide; peptides comprising an IR700 molecule conjugated to a lysine corresponding to position 145 (K145) of SEQ ID NO: 2, wherein the percent area of the conjugated EIC peak is at least at or about 8% of the total area of EIC peaks of the corresponding modified and unmodified polypeptide.

In some of any embodiments, trypsin digestion of the composition further produces mass spectra comprising extracted ion chromatogram (EIC) peaks corresponding to one or more of: peptides comprising an IR700 molecule conjugated to a lysine corresponding to position 449 (K449) of SEQ ID NO: 1, wherein the percent area of the conjugated EIC peak is at least at or about 5% of the total area of EIC peaks of the corresponding modified and unmodified polypeptide; and/or peptides comprising an IR700 molecule conjugated to a lysine corresponding to position 336 (K336) of SEQ ID NO: 1, wherein the percent area of the conjugated EIC peak is at least at or about 3.5% of the total area of EIC peaks of the corresponding modified and unmodified polypeptide. In some of any embodiments, trypsin digestion of the composition further produces mass spectra comprising extracted ion chromatogram (EIC) peaks corresponding to one or more of: peptides comprising an IR700 molecule conjugated to a lysine corresponding to position 5 (K5) of SEQ ID NO: 1, wherein the percent area of the conjugated EIC peak is at least at or about 2% of the total area of EIC peaks of the corresponding modified and unmodified polypeptide; peptides comprising an IR700 molecule conjugated to a lysine corresponding to position 75 (K75) of SEQ ID NO: 1, wherein the percent area of the conjugated EIC peak is at least at or about 2% of the total area of EIC peaks of the corresponding modified and unmodified polypeptide; peptides comprising an IR700 molecule conjugated to a lysine corresponding to position 248 (K248) of SEQ ID NO: 1, wherein the percent area of the conjugated EIC peak is at least at or about 0.5% of the total area of EIC peaks of the corresponding modified and unmodified polypeptide; peptides comprising an IR700 molecule conjugated to a lysine corresponding to position 328 (K328) of SEQ ID NO: 1, wherein the percent area of the conjugated EIC peak is at least at or about 0.5% of the total area of EIC peaks of the corresponding modified and unmodified polypeptide; peptides comprising an IR700 molecule conjugated to a lysine corresponding to position 107 (K107) of SEQ ID NO: 2, wherein the percent area of the conjugated EIC peak is at least at or about 2% of the total area of EIC peaks of the corresponding modified and unmodified polypeptide; peptides comprising an IR700 molecule conjugated to a lysine corresponding to position 188 (K188) of SEQ ID NO: 2, wherein the percent area of the conjugated EIC peak is at least at or about 0.5% of the total area of EIC peaks of the corresponding modified and unmodified polypeptide; peptides comprising an IR700 molecule conjugated to a lysine corresponding to position 190 (K190) of SEQ ID NO: 2, wherein the percent area of the conjugated EIC peak is at least at or about 2% of the total area of EIC peaks of the corresponding modified and unmodified polypeptide; and/or peptides comprising an IR700 molecule conjugated to a lysine corresponding to position 207 (K207) of SEQ ID NO: 2, wherein the percent area of the conjugated EIC peak is at least at or about 0.5% of the total area of EIC peaks of the corresponding modified and unmodified polypeptide.

Also provided herein are compositions that include a plurality of conjugates, wherein the conjugates comprise IR700 conjugated to cetuximab, and wherein trypsin digestion of the composition produces peptides that generate mass spectra comprising extracted ion chromatogram (EIC) peaks corresponding to peptides comprising an IR700 molecule conjugated to a lysine corresponding to position 5 (K5) of SEQ ID NO: 1, wherein the percent area of the conjugated EIC peak is at least at or about 2.5% of the total area of EIC peaks of the corresponding modified and unmodified polypeptide; peptides comprising an IR700 molecule conjugated to a lysine corresponding to position 75 (K75) of SEQ ID NO: 1, wherein the percent area of the conjugated EIC peak is at least at or about 2.5% of the total area of EIC peaks of the corresponding modified and unmodified polypeptide; peptides comprising an IR700 molecule conjugated to a lysine corresponding to position 215 (K215) of SEQ ID NO: 1, wherein the percent area of the conjugated EIC peak is at least at or about 9%, of the total area of EIC peaks of the corresponding modified and unmodified polypeptide; peptides comprising an IR700 molecule conjugated to a lysine corresponding to position 248 (K248) of SEQ ID NO: 1, wherein the percent area of the conjugated EIC peak is at least at or about 0.5% of the total area of EIC peaks of the corresponding modified and unmodified polypeptide; and/or peptides comprising an IR700 molecule conjugated to a lysine corresponding to position 292 (K292) of SEQ ID NO: 1, wherein the percent area of the conjugated EIC peak is about at least at or about 8.5%, of the total area of EIC peaks of the corresponding modified and unmodified polypeptide; peptides comprising an IR700 molecule conjugated to a lysine corresponding to position 328 (K328) of SEQ ID NO: 1, wherein the percent area of the conjugated EIC peak is at least at or about 0.5% of the total area of EIC peaks of the corresponding modified and unmodified polypeptide; peptides comprising an IR700 molecule conjugated to a lysine corresponding to position 336 (K336) of SEQ ID NO: 1, wherein the percent area of the conjugated EIC peak is at least at or about 4.5% of the total area of EIC peaks of the corresponding modified and unmodified polypeptide; peptides comprising an IR700 molecule conjugated to a lysine corresponding to position 416 (K416) of SEQ ID NO: 1, wherein the percent area of the conjugated EIC peak is at least at or about 9%, of the total area of EIC peaks of the corresponding modified and unmodified polypeptide; peptides comprising an IR700 molecule conjugated to a lysine corresponding to position 449 (K449) of SEQ ID NO: 1, wherein the percent area of the conjugated EIC peak is at least at or about 7% of the total area of EIC peaks of the corresponding modified and unmodified polypeptide; peptides comprising an IR700 molecule conjugated to a lysine corresponding to position 107 (K107) of SEQ ID NO: 2, wherein the percent area of the conjugated EIC peak is at least at or about 2.5% of the total area of EIC peaks of the corresponding modified and unmodified polypeptide; peptides comprising an IR700 molecule conjugated to a lysine corresponding to position 145 (K145) of SEQ ID NO: 1, wherein the percent area of the conjugated EIC peak is at least at or about 8.5%, of the total area of EIC peaks of the corresponding modified and unmodified polypeptide; peptides comprising an IR700 molecule conjugated to a lysine corresponding to position 188 (K188) of SEQ ID NO: 2, wherein the percent area of the conjugated EIC peak is at least at or about 1% of the total area of EIC peaks of the corresponding modified and unmodified polypeptide; peptides comprising an IR700 molecule conjugated to a lysine corresponding to position 190 (K190) of SEQ ID NO: 2, wherein the percent area of the conjugated EIC peak is at least at or about 2.5% of the total area of EIC peaks of the corresponding modified and unmodified polypeptide; and peptides comprising an IR700 molecule conjugated to a lysine corresponding to position 207 (K207) of SEQ ID NO: 2, wherein the percent area of the conjugated EIC peak is at least at or about 1% of the total area of EIC peaks of the corresponding modified and unmodified polypeptide.

In some of any embodiments, the percent area of the conjugated EIC peak is about 3.8±1% for the peptides comprising an IR700 molecule conjugated to a lysine corresponding to position 5 (K5) of SEQ ID NO: 1; the percent area of the conjugated EIC peak is about 3.5±1% for the peptides comprising an IR700 molecule conjugated to a lysine corresponding to position 75 (K75) of SEQ ID NO: 1; the percent area of the conjugated EIC peak is about 10.0±1%, for the peptides comprising an IR700 molecule conjugated to a lysine corresponding to position 215 (K215) of SEQ ID NO: 1; the percent area of the conjugated EIC peak is about 1.7±1% for the peptides comprising an IR700 molecule conjugated to a lysine corresponding to position 248 (K248) of SEQ ID NO: 1; the percent area of the conjugated EIC peak is about 10.2±1% for the peptides comprising an IR700 molecule conjugated to a lysine corresponding to position 292 (K292) of SEQ ID NO: 1; the percent area of the conjugated EIC peak is about 1.3±1% for the peptides comprising an IR700 molecule conjugated to a lysine corresponding to position 328 (K328) of SEQ ID NO: 1; the percent area of the conjugated EIC peak about 5.9±1% for the peptides comprising an IR700 molecule conjugated to a lysine corresponding to position 336 (K336) of SEQ ID NO: 1; the percent area of the conjugated EIC peak is about 11.2±1%, for the peptides comprising an IR700 molecule conjugated to a lysine corresponding to position 416 (K416) of SEQ ID NO: 1; the percent area of the conjugated EIC peak about 7.6±1% for the peptides comprising an IR700 molecule conjugated to a lysine corresponding to position 449 (K449) of SEQ ID NO: 1; the percent area of the conjugated EIC peak is about 3.4±1% for the peptides comprising an IR700 molecule conjugated to a lysine corresponding to position 107 (K107) of SEQ ID NO: 2; the percent area of the conjugated EIC peak is about 9.3±1%, for the peptides comprising an IR700 molecule conjugated to a lysine corresponding to position 145 (K145) of SEQ ID NO: 2; the percent area of the conjugated EIC peak is about 2.1±1% for the peptides comprising an IR700 molecule conjugated to a lysine corresponding to position 188 (K188) of SEQ ID NO: 2; the percent area of the conjugated EIC peak is about 3.5±1% for the peptides comprising an IR700 molecule conjugated to a lysine corresponding to position 190 (K190) of SEQ ID NO: 2; and the percent area of the conjugated EIC peak is about 2±1% for the peptides comprising an IR700 molecule conjugated to a lysine corresponding to position 207 (K207) of SEQ ID NO: 2.

In some of any embodiments, the peptides comprising an IR700 molecule conjugated to a lysine comprise one or more amino acid sequences selected from among: the sequence of amino acids corresponding to amino acids 1-38, amino acids 72-81 amino acids 213-216, amino acids 225-250, amino acids 291-294, amino acids 325-336, amino acids 329-340, amino acids 412-418, and amino acids 442-449 of SEQ ID NO: 1, the sequence of amino acids corresponding to amino acids 104-108, amino acids 143-149, amino acids 184-190, amino acids 189-207, and amino acids 191-211 of SEQ ID NO: 2.

Provided herein is a conjugate comprising a cetuximab antibody conjugated to an IR700, where at least one molecule of the IR700 is linked to a lysine (K) in a light chain of the cetuximab antibody. In one aspect, the at least one molecule of the IR700 may be linked to a lysine at a position selected from the group consisting of K107, K145, K188, K190, and K207 in the light chain. In any of the provided embodiments, the at least one molecule of the IR700 may be linked to K145 in the light chain. In any of the provided embodiments, the cetuximab antibody may be linked to the IR700 at two or more lysine positions in one or both light chains of the cetuximab antibody. In any of the provided embodiments, the cetuximab antibody may be linked to the IR700 at K145 in one or both light chains of the cetuximab antibody. In any of the provided embodiments, the cetuximab antibody may be linked to the IR700 at K145 in a light chain of the cetuximab antibody and at least one other lysine position in a light chain or a heavy chain of the cetuximab antibody.

Provided herein is a conjugate comprising a cetuximab antibody conjugated to an IR700, where at least one molecule of the IR700 is linked to a lysine (K) in a heavy chain of the cetuximab antibody. In one aspect, the at least one molecule of the IR700 may be linked to a lysine at a position selected from the group consisting of K5, K75, K215, K248, K292, K328, K336, K416, and K449 in the heavy chain. In any of the provided embodiments, the at least one molecule of the IR700 may be linked to a lysine at a position selected from the group consisting of K215, K292, K336, K416, and K449 in the heavy chain. In any of the provided embodiments, the cetuximab antibody may be linked to the IR700 at two or more lysine positions in one or both heavy chains of the cetuximab antibody. In any of the provided embodiments, the cetuximab antibody may be linked to the IR700 at three or more lysine positions in one or both heavy chains of the cetuximab antibody. In any of the provided embodiments, the cetuximab antibody may be linked to the IR700 at K215, K292, K336, K416, and/or K449 in one or both heavy chains of the cetuximab antibody. In any of the provided embodiments, the cetuximab antibody may be linked to the IR700 at K215, K292, K336, K416, and/or K449 in a heavy chain of the cetuximab antibody and at least one other lysine position in a light chain or a heavy chain of the cetuximab antibody. In any of the provided embodiments, the cetuximab antibody may be linked to the IR700 at K145 in one or both light chains of the cetuximab antibody.

Provided herein is a conjugate comprising a cetuximab antibody conjugated to an IR700, where at least one molecule of the IR700 is linked to a lysine (K) in a light chain of the cetuximab antibody, and at least one molecule of the IR700 is linked to a lysine (K) in a heavy chain of the cetuximab antibody. In one aspect, the lysine in the light chain may be selected from the group consisting of K107, K145, K188, K190, and K207. In any of the provided embodiments, the lysine in the heavy chain may be selected from the group consisting of K5, K75, K215, K248, K292, K328, K336, K416, and K449. In any of the provided embodiments, the cetuximab antibody may be linked to the IR700 at K145 in one or both light chains of the cetuximab antibody. In any of the provided embodiments, the cetuximab antibody may be linked to the IR700 at K215, K292, K336, K416, and/or K449 in one or both heavy chains of the cetuximab antibody. In any of the provided embodiments, the cetuximab antibody may be linked to the IR700 at K145 in one or both light chains of the cetuximab antibody, and at K215, K292, K416, and K449 in one or both heavy chains of the cetuximab antibody.

In any of the provided embodiments, the ratio of the IR700 molecule to the cetuximab antibody of the conjugate may be 1:1, 2:1, 3:1, or 4:1. In any of the provided embodiments, the conjugate is capable of being activated by irradiation with a wavelength between about 600 nm and about 850 nm (e.g., 690 nm±50 nm) and thereby acquire a cell killing activity.

In some of any embodiments, provided herein is a composition comprising the conjugate of any of the preceding embodiments and a pharmaceutically acceptable excipient. In some of any embodiments, provided herein is a composition comprising a population of cetuximab antibody molecules, where at least 50%, 60%, 70%, 80%, 90%, or more than 90% of the cetuximab antibody molecules have at least one molecule of an IR700 linked to a lysine (K) in a light chain of each cetuximab antibody molecule. In one aspect, at least 50%, 60%, 70%, 80%, 90%, or more than 90% of the cetuximab antibody molecules may have at least one molecule of the IR700 linked to K107, K145, K188, K190, and/or K207 in one or both light chains of each cetuximab antibody molecule. In any of the provided embodiments, the predominant light chain lysine position conjugated to the IR700 in the population may be K145. In any of the provided embodiments, when the cetuximab antibody molecules are analyzed by denatured mass spectrometry, peptides containing IR700 conjugation at light chain K145 may be more abundant than peptides containing IR700 conjugated to other light chain lysine positions.

In some of any embodiments, provided herein is a composition comprising a population of cetuximab antibody molecules, where at least 50%, 60%, 70%, 80%, 90%, or more than 90% of the cetuximab antibody molecules have at least one molecule of an IR700 linked to a lysine (K) in a heavy chain of each cetuximab antibody molecule. In one aspect, at least 50%, 60%, 70%, 80%, 90%, or more than 90% of the cetuximab antibody molecules may have at least one molecule of the IR700 linked to K5, K75, K215, K248, K292, K328, K336, K416, and/or K449 in one or both heavy chains of each cetuximab antibody molecule. In any of the provided embodiments, the predominant heavy chain lysine position conjugated to the IR700 in the population may be one or more of K215, K292, K336, K416, and K449. In any of the provided embodiments, when the cetuximab antibody molecules are analyzed by mass spectrometry, peptides containing IR700 conjugation at heavy chain K215, K292, K336, K416, or K449 may be more abundant than peptides containing IR700 conjugated to other heavy chain lysine positions. In any of the provided embodiments, at least 70%, 80%, 90%, or more than 90% of the cetuximab antibody molecules may have at least one molecule of the IR700 linked to two or more lysines in one or both heavy chains of each cetuximab antibody molecule.

In any of the provided embodiments, no more than about 20% of the cetuximab antibody molecules may be unconjugated with IR700. In any of the provided embodiments, less than 10% of the cetuximab antibody molecules may be unconjugated with the IR700, and/or the percentage of free dye in the composition may be less than 3%. In any of the provided embodiments, the ratio of the IR700 molecules in the composition to the cetuximab antibody molecules in the population may be about 2:1, about 2.5:1, or about 3:1.

In some of any embodiments, provided herein is a composition comprising a population of cetuximab antibody molecules, where at least 50%, 60%, 70%, 80%, 90%, or more than 90% of the cetuximab antibody molecules have at least one molecule of an IR700 linked to a lysine (K) in a light chain or a heavy chain of each cetuximab antibody molecule. In one aspect, at least 50%, 60%, 70%, 80%, 90%, or more than 90% of the cetuximab antibody molecules may have at least one molecule of the IR700 linked to K107, K145, K188, K190, and/or K207 in one or both light chains or linked to K5, K75, K215, K248, K292, K328, K336, K416, and/or K449 in one or both heavy chains of each cetuximab antibody molecule. In one aspect, at least 50%, 60%, 70%, 80%, 90%, or more than 90% of the cetuximab antibody molecules may have at least one molecule of the IR700 linked to K145 in one or both light chains or linked to K215, K292, K336, K416, or K449 in one or both heavy chains of each cetuximab antibody molecule.

In some of any embodiments, provided herein is a composition comprising a population of cetuximab antibody molecules, where at least 50%, 60%, 70%, 80%, 90%, or more than 90% of the cetuximab antibody molecules have at least one molecule of an IR700 linked to a lysine (K) in a light chain and at least one molecule of the IR700 linked to a lysine in a heavy chain of each cetuximab antibody molecule. In one aspect, at least 50%, 60%, 70%, 80%, 90%, or more than 90% of the cetuximab antibody molecules may have at least one molecule of the phthalocyanine IR700 linked to K107, K145, K188, K190, and/or K207 in one or both light chains and at least one molecule of the IR700 linked to K5, K75, K215, K248, K292, K328, K336, K416, and/or K449 in one or both heavy chains of each cetuximab antibody molecule. In one aspect, at least 50%, 60%, 70%, 80%, 90%, or more than 90% of the cetuximab antibody molecules may have at least one molecule of the phthalocyanine IR700 linked to K145 in one or both light chains and at least one molecule of the IR700 linked to K215, K292, K336, K416, or K449 in one or both heavy chains of each cetuximab antibody molecule.

Provided herein is a composition comprising a population of cetuximab antibody molecules, where no more than about 20% of the cetuximab antibody molecules are unconjugated with an IR700 at a lysine (K) in a light chain or a heavy chain of the cetuximab antibody molecules. In one aspect, less than 15% of the cetuximab antibody molecules may be unconjugated with the IR700. In one aspect, less than 10% of the cetuximab antibody molecules may be unconjugated with the IR700. In any of the provided embodiments, the percentage of free dye in the composition among all dye molecules in the composition may be less than 3%, about 2%, about 1%, or about 0.5%. In any of the provided embodiments, the ratio of the IR700 molecules in the composition to the cetuximab antibody molecules in the population may be about 2:1, about 2.5:1, or about 3:1.

Provided herein is a composition comprising a population of cetuximab antibody molecules, where one or more cetuximab antibody molecules are conjugated with an IR700 at a lysine (K) in a light chain or a heavy chain of the one or more cetuximab antibody molecules, the ratio of the IR700 molecules in the composition to the cetuximab antibody molecules in the population is about 2:1, about 2.5:1, or about 3:1, less than 10% of the cetuximab antibody molecules are unconjugated with the IR700, and the percentage of free dye in the composition among all dye molecules in the composition is less than about 0.5%.

Provided herein is a composition comprising SEQ ID NO: 1, where lysine 145 of SEQ ID NO: 1 is conjugated to an IR700. Provided herein is a composition comprising SEQ ID NO: 2, where lysine 215, lysine 292, lysine 416, and/or lysine 449 of SEQ ID NO: 2 is or are conjugated to an IR700.

Provided herein is a composition comprising SEQ ID NO: 1 and SEQ ID No: 2, where lysine 145 of SEQ ID NO: 1 is conjugated to an IR700 and lysine 215, lysine 292, lysine 416, and/or lysine 449 of SEQ ID NO: 2 is or are conjugated to an IR700. In one aspect, the composition may comprise SEQ ID NO: 1 and SEQ ID NO: 2 in the same molecule(s), where lysine 145 of SEQ ID NO: 1 may be conjugated to an IR700 and lysine 215, lysine 292, lysine 416, and/or lysine 449 of SEQ ID NO: 2 may be conjugated to an IR700. In any of the provided embodiments, the composition may comprise SEQ ID NO: 1 and SEQ ID NO: 2 in different molecules, where lysine 145 of SEQ ID NO: 1 may be conjugated to an IR700 and lysine 215, lysine 292, lysine 416, and/or lysine 449 of SEQ ID NO: 2 may be conjugated to an IR700.

Provided herein is a composition comprising a population of cetuximab antibody molecules, where less than 15% of the cetuximab antibody molecules are unconjugated with an IR700, the composition comprises less than 3% free IR700, and the percentage of free dye in the composition is substantially unchanged after storage for about 6 months.

Provided herein is a composition comprising a population of cetuximab antibody molecules conjugated with an IR700, where the composition comprises less than or less than about 0.6%, less than or less than about 0.5%, less than or less than about 0.4%, or less than or less than about 0.3% free dye. In one aspect, the composition may comprise at least or at least about 95%, 96% 97% or 98% monomer. In one aspect, the composition may comprise less than or less than about 5%, 4% or 3% high molecular weight species. In any of the provided embodiments, the composition may comprise less than or less than about 30%, 20%, 25%, 20%, 15%, 10%, 9%, 8%, 7%, 6%, or 5% unconjugated antibody.

In any of the provided embodiments, the percentage of free dye may be substantially unchanged after storage of the composition for 6 months in dark or reduced light conditions. In any of the provided embodiments, at least 70%, 80%, 90%, or more than 90% of the cetuximab antibody molecules may have at least one IR700 molecule conjugated to a lysine (K) in a light chain of each cetuximab antibody molecule. In any of the provided embodiments, the predominant light chain lysine position conjugated to the IR700 in the population may be K145. In any of the provided embodiments, at least 70%, 80%, 90%, or more than 90% of the population may have at least one IR700 molecule conjugated to a lysine (K) in a heavy chain of each cetuximab antibody molecule. In any of the provided embodiments, the predominant heavy chain lysine position conjugated to the IR700 in the population may be one or more of K215, K292, K336, K416, and K449.

Provided herein is a composition comprising a population of cetuximab antibody molecules, where between about 9% and about 10% of total peptides of the cetuximab antibody molecules analyzed by mass spectrometry are peptides that contain IR700 conjugation at light chain lysine 145 (K145). Provided herein is a composition comprising a population of cetuximab antibody molecules, where between about 9% and about 11% of total peptides of the cetuximab antibody molecules analyzed by mass spectrometry are peptides that contain IR700 conjugation at heavy chain lysine 215 (K215). Provided herein is a composition comprising a population of cetuximab antibody molecules, where between about 9% and about 11% of total peptides of the cetuximab antibody molecules analyzed by mass spectrometry are peptides that contain IR700 conjugation at heavy chain lysine 292 (K292). Provided herein is a composition comprising a population of cetuximab antibody molecules, where between about 10% and about 12% of total peptides of the cetuximab antibody molecules analyzed by mass spectrometry are peptides that contain IR700 conjugation at heavy chain lysine 416 (K416). Provided herein is a composition comprising a population of cetuximab antibody molecules, where between about 7% and 9% of total peptides of the cetuximab antibody molecules analyzed by mass spectrometry are peptides that contain IR700 conjugation at heavy chain lysine 449 (K449). Provided herein is a composition comprising a population of cetuximab antibody molecules, where between about 5% and 7% of total peptides of the cetuximab antibody molecules analyzed by mass spectrometry are peptides that contain IR700 conjugation at heavy chain lysine 336 (K336). Provided herein is a composition comprising a population of cetuximab antibody molecules, where between about 9% and 11% of total peptides of the cetuximab antibody molecules analyzed by mass spectrometry are peptides that contain IR700 conjugation at light chain lysine 145 (K145); between about 9% and 11% of total peptides of the cetuximab antibody molecules analyzed by mass spectrometry are peptides that contain IR700 conjugation at heavy chain lysine 215 (K215); between about 9% and 11% of total peptides of the cetuximab antibody molecules analyzed by mass spectrometry are peptides that contain IR700 conjugation at heavy chain lysine 292 (K292); between about 10% and 12% of total peptides of the cetuximab antibody molecules analyzed by mass spectrometry are peptides that contain IR700 conjugation at heavy chain lysine 416 (K416); between about 7% and 9% of total peptides of the cetuximab antibody molecules analyzed by mass spectrometry are peptides that contain IR700 conjugation at heavy chain lysine 449 (K449); and/or about between about 5% and 7% of total peptides of the cetuximab antibody molecules analyzed by mass spectrometry are peptides that contain IR700 conjugation at heavy chain lysine 336 (K336). In any of the provided embodiments, between about 3% and 4% of total peptides of the cetuximab antibody molecules analyzed by mass spectrometry may be peptides that contain IR700 conjugation at light chain lysine 107 (K107); between about 1% and 3% of total peptides of the cetuximab antibody molecules analyzed by mass spectrometry may be peptides that contain IR700 conjugation at light chain lysine 188 (K188); between about 3% and 4% of total peptides of the cetuximab antibody molecules analyzed by mass spectrometry may be peptides that contain IR700 conjugation at light chain lysine 190 (K190); between about 1% and 3% of total peptides of the cetuximab antibody molecules analyzed by mass spectrometry may be peptides that contain IR700 conjugation at light chain lysine 207 (K207); between about 3% and 4% of total peptides of the cetuximab antibody molecules analyzed by mass spectrometry may be peptides that contain IR700 conjugation at heavy chain lysine 5 (K5); between about 3% and 4% of total peptides of the cetuximab antibody molecules analyzed by mass spectrometry may be peptides that contain IR700 conjugation at heavy chain lysine 75 (K75); between about 1% and 2% of total peptides of the cetuximab antibody molecules analyzed by mass spectrometry may be peptides that contain IR700 conjugation at heavy chain lysine 248 (K248); and/or between about 1% and 2% of total peptides of the cetuximab antibody molecules analyzed by mass spectrometry may be peptides that contain IR700 conjugation at heavy chain lysine 328 (K328).

Provided herein is a composition comprising a population of cetuximab antibody molecules conjugated to IR700, where: the ratio of IR700 conjugated to lysines in the population of cetuximab antibody molecules is about 2:1 to about 1:2, optionally about 1:1, between positions lysine 145 (K145) in the light chain and lysine 215 (K215) in the heavy chain; the ratio of IR700 conjugated to lysines in the population of cetuximab antibody molecules is about 2:1 to about 1:2, optionally about 1:1, between positions lysine 145 (K145) in the light chain and lysine 292 (K292) in the heavy chain; the ratio of IR700 conjugated to lysines in the population of cetuximab antibody molecules is about 2:1 to about 1:2, optionally about 1:1, between positions lysine 145 (K145) in the light chain and lysine 336 (K336) in the heavy chain; the ratio of IR700 conjugated to lysines in the population of cetuximab antibody molecules is about 2:1 to about 1:2, optionally about 1:1, between positions lysine 145 (1(145) in the light chain and lysine 416 (K416) in the heavy chain; and/or the ratio of IR700 conjugated to lysines in the population of cetuximab antibody molecules is about 2:1 to about 1:2, optionally about 1:1, between positions lysine 145 (K145) in the light chain and lysine 449 (K449) in the heavy chain. Provided herein is a composition comprising a population of cetuximab antibody molecules conjugated to IR700, where the ratio of IR700 conjugated to lysines in the population of cetuximab antibody molecules is about 2:1 to about 1:2, optionally about 1:1, between positions lysine 215 (K215) in the heavy chain and lysine 292 (K292) in the heavy chain; the ratio of IR700 conjugated to lysines in the population of cetuximab antibody molecules is about 2:1 to about 1:2, optionally about 1:1, between positions lysine 215 (1(215) in the heavy chain and lysine 336 (K336) in the heavy chain; the ratio of IR700 conjugated to lysines in the population of cetuximab antibody molecules is about 2:1 to about 1:2, optionally about 1:1, between positions lysine 215 (1(215) in the heavy chain and lysine 416 (K416) in the heavy chain; and/or the ratio of IR700 conjugated to lysines in the population of cetuximab antibody molecules is about 2:1 to about 1:2, optionally about 1:1, between positions lysine 215 (K215) in the heavy chain and lysine 449 (K449) in the heavy chain. Provided herein is a composition comprising a population of cetuximab antibody molecules conjugated to IR700, where the ratio of IR700 conjugated to lysines in the population of cetuximab antibody molecules is about 2:1 to about 1:2, optionally about 1:1, between positions lysine 292 (K292) in the heavy chain and lysine 336 (K336) in the heavy chain; the ratio of IR700 conjugated to lysines in the population of cetuximab antibody molecules is about 2:1 to about 1:2, optionally about 1:1, between positions lysine 292 (K292) in the heavy chain and lysine 416 (K416) in the heavy chain; and/or the ratio of IR700 conjugated to lysines in the population of cetuximab antibody molecules is about 2:1 to about 1:2, optionally about 1:1, between positions lysine 292 (K292) in the heavy chain and lysine 449 (K449) in the heavy chain. Provided herein is a composition comprising a population of cetuximab antibody molecules conjugated to IR700, where the ratio of IR700 conjugated to lysines in the population of cetuximab antibody molecules is about 2:1 to about 1:2, optionally about 1:1, between positions lysine 336 (K336) in the heavy chain and lysine 416 (K416) in the heavy chain; and/or the ratio of IR700 conjugated to lysines in the population of cetuximab antibody molecules is about 2:1 to about 1:2, optionally about 1:1, between positions lysine 336 (K336) in the heavy chain and lysine 449 (K449) in the heavy chain. Provided herein is a composition comprising a population of cetuximab antibody molecules conjugated to IR700, where the ratio of IR700 conjugated to lysines in the population of cetuximab antibody molecules is about 2:1 to about 1:2, optionally about 1:1, between positions lysine 416 (K416) in the heavy chain and lysine 449 (K449) in the heavy chain.

Provided herein is a composition comprising a population of cetuximab antibody molecules conjugated to IR700, where the ratio of IR700 conjugated to lysines in the population of cetuximab antibody molecules is about 1:1:1:1 among positions lysine 145 (K145) in the light chain, lysine 215 (K215) in the heavy chain, lysine 292 (K292) in the heavy chain, and lysine 416 (K416) in the heavy chain.

Provided herein is a composition comprising a population of cetuximab antibody molecules conjugated to IR700, where the ratio of IR700 conjugated to lysines in the population of cetuximab antibody molecules is about 1:1:1:1:1:1 among positions lysine 145 (K145) in the light chain, lysine 215 (1(215) in the heavy chain, lysine 292 (K292) in the heavy chain, lysine 336 (K336) in the heavy chain, lysine 416 (K416) in the heavy chain, and lysine 449 (K449) in the heavy chain.

In any of the provided embodiments, the ratio of IR700 conjugated to lysines in the population of cetuximab antibody molecules may be measured by mass spectrometry.

Provided herein is a method of killing a tumor or cancer cell, comprising administering a pharmaceutical composition comprising the conjugate or composition of any of the preceding embodiments to a site at or proximal to the tumor or cancer cell; and irradiating an area proximal to the tumor cell at a wavelength of about 600 nm to about 850 nm at a dose of from about 25 J cm⁻² to about 400 J cm⁻² or from about 25 J/cm of fiber length to about 500 J/cm of fiber length, thereby killing the tumor or cancer cell. Provided herein is a method of treating a disease or condition, e.g., a tumor or cancer, in a subject, comprising administering a pharmaceutical composition comprising the conjugate or composition of any of the preceding embodiments to the subject; and irradiating an area proximal to a lesion (e.g., due to the tumor or cancer) in the subject at a wavelength of about 600 nm to about 850 nm at a dose of from about 25 J cm⁻² to about 400 J cm⁻² or from about 25 J/cm of fiber length to about 500 J/cm of fiber length, thereby treating the disease or condition. In any of the provided embodiments, the method may further comprise providing the pharmaceutical composition prior to the administering step. In any of the provided embodiments, the irradiating step may be carried out at a wavelength of 690±50 nm or at a wavelength of or about 690±20 nm. In any of the provided embodiments, the irradiating step may be carried out at a wavelength of about 690 nm. In any of the provided embodiments, the tumor may be a carcinoma of the bladder, pancreas, colon, ovary, lung, breast, stomach, prostate, cervix, esophagus or head and neck. In any of the provided embodiments, the cancer may be located at the head and neck, breast, liver, colon, ovary, prostate, pancreas, brain, cervix, bone, skin, eye, bladder, stomach, esophagus, peritoneum, or lung. In any of the provided embodiments, the cancer may be a cancer located at the head and neck.

Provided herein is a method of manufacturing a stable conjugate comprising a) contacting a cetuximab antibody with an IR700 under conditions to produce a conjugate comprising the IR700 linked to one or more lysines of the cetuximab antibody selected from the group consisting of K145 (light chain), K215 (heavy chain), K292 (heavy chain), K336 (heavy chain), K416 (heavy chain), and K449 (heavy chain); b) subjecting the conjugate to a step during and/or subsequent to conjugation which substantially reduces the IR700 non-specifically associated with the cetuximab antibody; c) formulating the conjugate in a pharmaceutically acceptable buffer, where in each of steps a)-c) the only light to which the dye and conjugate are exposed has a wavelength within a range of about 400 nm to about 650 nm or has an intensity of less than 500 lux. In one aspect, step b) may comprise subjecting the conjugate to a quenching reaction. In any of the provided embodiments, step b) may comprise subjecting the conjugate to a glycine quenching reaction after completion of the conjugation reaction between the IR700 and the cetuximab antibody. In any of the provided embodiments, the quenching reaction may be performed overnight or for a duration of greater than about 6 hours.

Provided herein in some of any embodiments is the stable conjugate manufactured by any of the preceding embodiments. In any of the provided embodiments, the conjugate may comprise a population of cetuximab antibody molecules conjugated to IR700, where the ratio of IR700 conjugated to lysines of cetuximab antibody molecules in the population may be about 1:1:1:1 among positions lysine 145 (K145) in the light chain, lysine 215 (K215) in the heavy chain, lysine 292 (K292) in the heavy chain, and lysine 416 (K416) in the heavy chain.

### BRIEF DESCRIPTION OF THE DRAWINGS

FIG. 1 shows the results of a photoimmunotherapy (PIT) assay, comparing the BxPC3 cell-killing activities of three batches of the cetuximab-IR700 conjugate relative to a reference standard.

FIG. 2 shows the results of size-exclusion HPLC (SEC-HPLC) of three batches of the cetuximab-IR700 conjugate, showing all three lots exhibited at least 97% monomer, less than 3% high molecular weight species (HMW), and less than or equal to 0.3% free IR700 dye (e.g., unconjugated dye).

### DETAILED DESCRIPTION

Provided herein are conjugates of the phthalocyanine dye IR700 and a cetuximab antibody (cetuximab-IR700) and compositions, including pharmaceutical compositions containing such conjugates.

In some embodiments, the conjugates and preparation of conjugates disclosed herein provide increased uniformity, stability, activity, and/or improved consistency of compositions, including pharmaceutical compositions. In some embodiments, the cetuximab-IR700 conjugates contain a cetuximab antibody that is modified by conjugation to the IR700 dye at specific amino acid positions or specific amino acid residues, including at specific lysine (K) positions or residues. Also provided are cetuximab-IR700 conjugates and compositions containing such conjugates that result in a composition comprising a population of certain peptides and modifications, such as conjugation of IR700 at a specific lysine positions or residues, when digested with an endoprotease, such as trypsin. Also provided are cetuximab-IR700 conjugates and compositions containing such conjugates that exhibit particular mass spectra, when digested with an endoprotease, such as trypsin, and analyzed or assessed by mass spectrometry, such as liquid chromatography/mass spectrometry (LC/MS). Also provided are methods and uses, such as methods of treatment or therapeutic uses, related to any of the provided conjugates or compositions. Also provided are methods of generating any of the provided cetuximab-IR700 conjugates and/or compositions comprising the cetuximab-IR700 conjugates.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. IR700 PHTHALOCYANINE DYE AND CONJUGATES THEREOF

The conjugates provided herein include a phthalocyanine dye molecule, such as IR700 conjugated to an epidermal growth factor receptor (EGFR)-binding antibody, such as cetuximab, via a linker group. In one aspect, the conjugate is of Formula I:

A-[(L)ₙ-D]ₚ (I)

wherein:
A is a cetuximab or antibody directed against EGFR;
L is an independently selected linker for each p;
n is 1 or 2;
D is an independently selected hydrophilic phthalocyanine dye for each p; and
p is independently 1, 2, 3, 4, 5, generally 1 to 3.

In some embodiments, the phthalocyanine dye containing the reactive group is IR700 NHS ester, such as IRDye 700DX NHS ester (LiCor 929-70010, 929-70011). In some embodiments, the dye is a compound having the following formula:

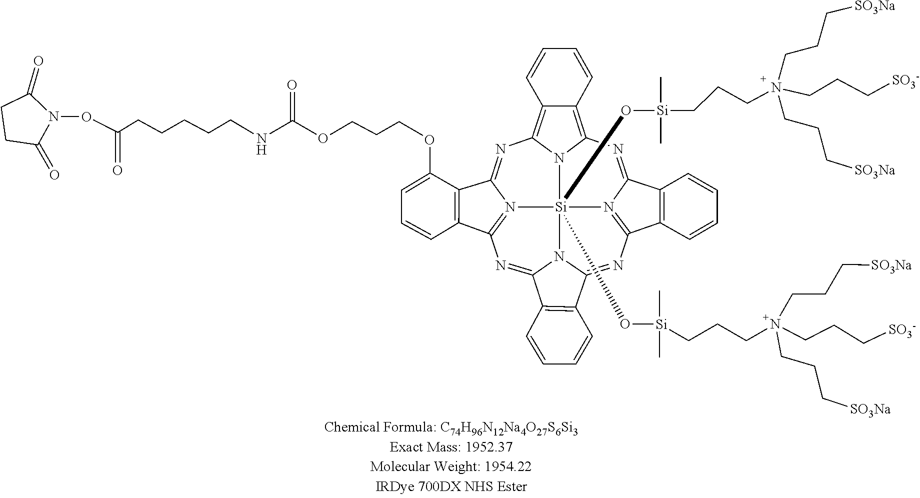

For purposes herein, the term “IR700” (also called IRDye 700 or IR700 dye) includes the above formula when the dye is conjugated such as to an antibody, e.g. via a reactive group. In some embodiments, an IR700 dye is a fluorescent dye that has an absorption and emission wavelengths in the near-infrared (NIR) spectrum typically between 680 nm and 800 nm.

The phthalocyanine dyes described herein can be made with commercially available starting material. The core structure is synthesized by condensation of two or more different diiminoisoindolines. Synthetic strategies using different dinitriles or diiminoisoindolines can lead to various degrees of substitution of the phthalocyanine and/or distribution of regioisomers. Exemplary synthetic schemes for generating the dyes are described in U.S. Pat. No. 7,005,518. Exemplary synthetic schemes for preparing and characterizing conjugates to dyes such as IR700 are described in WO 2017/031363.

In some embodiments, the IR700 phthalocyanine dye is conjugated to an antibody via a reactive group of the dye molecule. In some embodiments, the reactive group is an NHS ester.

In some embodiments, the cetuximab-IR700 conjugates contained in the compositions described herein contains a specific number of dye residues per antibody molecule that is from at or about 1 to at or about 3 dye molecules. In some embodiments, the number of dye molecule per antibody can be from at or about 2 to at or about 5, such as from at or about 2 to at or about 4, for example about 3 or 3. In some embodiments, the cetuximab-IR700 conjugate, the number of dye molecule per light chain of the antibody, can be at or about 1 or from at or about 1 to at or about 2. In some embodiments, the cetuximab-IR700 conjugate, the number of dye molecule per heavy chain of the antibody, can be at or about 1, at or about 2 or at or about 3 or at or about 4 or from at or about 1 to at or about 2 or from at or about 2 to at or about 3 or from at or about 2 to at or about 4.

### II. EPIDERMAL GROWTH FACTOR RECEPTOR (EGFR)-BINDING ANTIBODIES, CETUXIMAB AND CONJUGATES THEREOF

Provided herein are antibody conjugates that have one or more phthalocyanine dye molecules (such as IR700) conjugated at specific amino acid positions or residues in an antibody, in particular to lysine positions or residues of an antibody. In some embodiments, one or more dye molecules are conjugated to the constant regions of the light chain and/or the heavy chain of the antibody. Such conjugates retain the ability of the antibody to bind to its target antigen, and moreover the conjugate provides the target cell killing activity of the antibody-dye conjugate, for example following binding of the antibody-dye conjugate to the surface of a cell that expresses the target antigen, and illumination or irradiation with light.

In the conjugates and compositions provided herein, the position(s) of the dye molecules (e.g., the lysine (K) positions or residues of one or more chains or domains of the antibody that the IR700 is conjugated to) are consistent and repeatable, in some cases referring to or meaning that the selection of positions conjugated with IR700 on the heavy chain and the light chain as well as the proportionality of conjugation between positions is substantially constant from batch to batch of conjugated antibody. Consistency and repeatability of the conjugation positions provides a uniformity, predictability and constancy of activity and purity of the drug composition.

In some embodiments, the antibody in the provided conjugates or compositions is an antibody that targets or that binds, e.g., specifically binds, an epidermal growth factor receptor (EGFR; ErbB-1; HER1; EGF receptor), such that the antibody can bind EGFR on the surface of a cell. In one embodiment, the antibody is cetuximab. Cetuximab is a recombinant, human/mouse chimeric monoclonal antibody that binds specifically to the extracellular domain of EGFR. Cetuximab is composed of the Fv regions of a murine anti-EGFR antibody with human IgG1 heavy and kappa light chain constant regions. In some aspects, the antibody comprises a heavy chain comprising the sequence set forth in SEQ ID NO: 1. In some aspects, the antibody comprises a light chain comprising the sequence set forth in SEQ ID NO: 2. In some embodiments, the antibody comprises a heavy chain and a light chain comprising the sequence set forth in SEQ ID NOS: 1 and 2, respectively. In some embodiments, the provided conjugates comprise a cetuximab antibody, such as a cetuximab comprising a heavy chain and a light chain comprising the sequence set forth in SEQ ID NOS: 1 and 2, respectively. In some embodiments, the antibody is derived from cetuximab or is a variant or a derivative of cetuximab, such as an antigen-binding fragment or a modified version thereof, or a biosimilar, interchangeable or biobetter of cetuximab. Such antibodies also include copy biologicals and biogenerics of cetuximab.

An “antibody” as used herein is a polypeptide ligand comprising at least a light chain or heavy chain immunoglobulin variable region that specifically recognizes and binds an epitope of an antigen, such as a tumor-specific protein. Generally, antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

An “antibody” as used herein includes intact immunoglobulins and fragments of antibodies that exhibit antigen-binding, such as Fab fragments, Fab′ fragments, F(ab)′₂ fragments, single chain Fv proteins (“scFv”), and disulfide stabilized Fv proteins (“dsFv”). An scFv protein is a fusion protein in which a light chain variable region (V_{L}) of an immunoglobulin and a heavy chain variable region (V_{H}) of an immunoglobulin are bound by a linker, while in dsFvs, the chains have been mutated to introduce a disulfide bond to stabilize the association of the chains. The term also includes genetically engineered forms such as chimeric antibodies, for example, humanized murine antibodies, and heteroconjugate antibodies, such as bispecific antibodies. See also, Pierce Catalog and Handbook, 1994-1995 (Pierce Chemical Co., Rockford, Ill.); Kuby, J. Immunology, 3^{rd} Ed., W.H. Freeman & Co., New York, 1997

Typically, a naturally occurring immunoglobulin has heavy (H) chains and light (L) chains interconnected by disulfide bonds. There are two types of light chain, lambda (λ) and kappa (k). There are five main heavy chain classes, or isotypes, which determine the functional activity of an antibody molecule: IgM, IgD, IgG, IgA, and IgE. IgG antibodies are tetrameric proteins composed of two heavy chains and two light chains. The IgG heavy chain is composed of four immunoglobulin domains linked from N- to C-terminus in the order V_{H}-C_{H}1-C_{H}2-C_{H}3, referring to the heavy chain variable domain, heavy chain constant domain 1, heavy chain constant domain 2, and heavy chain constant domain 3 respectively (also referred to as V_{H}-Cγ1-Cγ2-Cγ3, referring to the heavy chain variable domain, constant gamma 1 domain, constant gamma 2 domain, and constant gamma 3 domain respectively for the IgG class). The IgG light chain is composed of two immunoglobulin domains linked from N- to C-terminus in the order V_{L}-C_{L}, referring to the light chain variable domain and the light chain constant domain respectively.

Each heavy and light chain contains a constant region and a variable region, also known as “domains.” In combination, the heavy and the light chain variable regions generally specifically bind the antigen. Light and heavy chain variable regions may contain a “framework” region interrupted by three hypervariable regions, also called “complementarity-determining regions” or “CDRs.” The extents of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are typically responsible for binding to an epitope of an antigen. The CDRs of each chain are typically referred to as CDR1, CDR2, and CDR3, numbered sequentially starting from the N-terminus, and are also generally identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities, such as different combining sites for different antigens, have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain (i.e., heavy chain variable region), including that of an Fv, scFv, dsFv or Fab. References to “V_{L}” or “VL” refer to the variable region of an immunoglobulin light chain (i.e., light chain variable region), including that of an Fv, scFv, dsFv or Fab.

Reference to “C_{H}” or “CH” refer to the constant region of an immunoglobulin heavy chain. References to “C_{L}” or “CL” refer to the constant region of an immunoglobulin light chain. The constant regions are so named because their amino acid sequence is relatively similar or identical between antibodies in the same isotype or heavy chain class. For example, constant regions of IgG antibodies may be highly homologous, even across species. Exemplary IgG isotypes include IgG1, IgG2, IgG3 and IgG4 and may be further divided into subclasses such as IgG2a and IgG2b. For example, the constant regions of human IgG1, IgG2, IgG3 and IgG4 show over 90% homology in amino acid sequence, with differences that are not randomly distributed. Much variation is found in the hinge region and N-terminal CH2 domain, whereas fewer amino acid differences are found in the other domains.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments. Other antibody fragments or multispecific antibodies formed from antibody fragments include a multivalent scFv, a bispecific scFv or an scFv-C_{H}3 dimer. Antibody fragments can be made by various techniques, including but not limited to proteolytic digestion of an intact antibody as well as production by recombinant host cells.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs, which generally confer antigen binding, from another species, such as a murine antibody that specifically binds mesothelin.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In some embodiments, the CDRs are from the donor immunoglobulin in a humanized immunoglobulin. Constant regions need not be present, but if they are, they may be substantially identical to human immunoglobulin constant regions, such as at least about 85-90%, such as about 95% or more identical. Hence, parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and CDRs from a human immunoglobulin. In some embodiments, the framework and the CDRs are from the same originating human heavy and/or the light chain amino acid sequence. However, frameworks from one human antibody can be engineered to include CDRs from a different human antibody. Parts of a human immunoglobulin may be substantially identical to corresponding parts of natural human immunoglobulin sequences.

Provided herein are antibodies, particularly antibodies that bind EGFR, conjugated to a phthalocyanine dye, such as IR700. In some embodiments, the antibody of the conjugate is cetuximab. In some embodiments, the cetuximab is conjugated to IR700 dye. In some embodiments, the cetuximab conjugated to IR700 has a heavy chain comprising SEQ ID NO: 1. In some embodiments, the cetuximab conjugated to IR700 has a light chain comprising SEQ ID NO: 2. In some embodiments, the cetuximab has a heavy chain and a light chain, comprising SEQ ID NOs: 1 and 2, respectively.

```
SEQ ID NO: 1
                10 20 30 40
QVQLKQSGPG LVQPSQSLSI TCTVSGFSLT NYGVHWVRQS
                50 60 70 80
PGKGLEWLGV IWSGGNTDYN TPFTSRLSIN KDNSKSQVFF
                90 100 110 120
KMNSLQSNDT AIYYCARALT YYDYEFAYWG QGTLVTVSAA
              130 140 150 160
STKGPSVFPL APSSKSTSGG TAALGCLVKD YFPEPVTVSW
              170 180 190 200
NSGALTSGVH TFPAVLQSSG LYSLSSVVTV PSSSLGTQTY
              210 220 230 240
ICNVNHKPSN TKVDKRVEPK SCDKTHTCPP CPAPELLGGP
              250 260 270 280
SVFLFPPKPK DTLMISRTPE VTCVVVDVSH EDPEVKFNWY
              290 300 310 320
VDGVEVHNAK TKPREEQYNS TYRVVSVLTV LHQDWLNGKE
              330 340 350 360
YKCKVSNKAL PAPIEKTISK AKGQPREPQV YTLPPSREEM
              370 380 390 400
TKNQVSLTCL VKGFYPSDIA VEWESNGQPE NNYKTTPPVL
              410 420 430 440
DSDGSFFLYS KLTVDKSRWQ QGNVFSCSVM HEALHNHYTQ
KSLSLSPGK
SEQ ID NO: 2
                10 20 30 40
DILLTQSPVI LSVSPGERVS FSCRASQSIG TNIHWYQQRT
                50 60 70 80
NGSPRLLIKY ASESISGIPS RFSGSGSGTD FTLSINSVES
                90 100 110 120
EDIADYYCQQ NNNWPTTFGA GTKLELKRTV AAPSVFIFPP
              130 140 150 160
SDEQLKSGTA SVVCLLNNFY PREAKVQWKV DNALQSGNSQ
              170 180 190 200
ESVTEQDSKD STYSLSSTLT LSKADYEKHK VYACEVTHQG
              210
LSSPVTKSFN RGA
```

As used herein, “sequence identity” between two polypeptide sequences indicates the percentage of amino acids that are identical between the sequences. “Sequence similarity” indicates the percentage of amino acids that either are identical or that represent conservative amino acid substitutions.

In some embodiments, a cetuximab disclosed herein has a sequence identity in the CDR regions of at least 60%, more preferably, at least 70% or 80%, still more preferably at least 90% and most preferably at least 95%, with one or more of the CDR regions in the heavy chain sequence set forth in SEQ ID NO: 1. In some embodiments, a cetuximab disclosed herein has a sequence identity in the CDR regions of at least 60%, more preferably, at least 70% or 80%, still more preferably at least 90% and most preferably at least 95%, with one or more of the CDR regions in the light chain sequence set forth in SEQ ID NO: 2. In some embodiments, a cetuximab disclosed herein has a sequence identity in the CDR regions of at least 60%, more preferably, at least 70% or 80%, still more preferably at least 90% and most preferably at least 95%, with one or more of the CDR regions in the heavy chain sequence set forth in SEQ ID NO: 1 and in the light chain sequence set forth in SEQ ID NO: 2.

In some embodiments, a cetuximab disclosed herein has a sequence similarity in the CDR regions of at least 80%, more preferably 90% and most preferably 95%, with one or more of the CDR regions in the heavy chain sequence set forth in SEQ ID NO: 1. In some embodiments, a cetuximab disclosed herein has a sequence similarity in the CDR regions of at least 80%, more preferably 90% and most preferably 95%, with one or more of the CDR regions in the light chain sequence set forth in SEQ ID NO: 2. In some embodiments, a cetuximab disclosed herein has a sequence similarity in the CDR regions of at least 80%, more preferably 90% and most preferably 95%, with one or more of the CDR regions in the heavy chain sequence set forth in SEQ ID NO: 1 and in the light chain sequence set forth in SEQ ID NO: 2.

In some embodiments, the antibody is a variant or derivative of cetuximab (e.g., comprising a heavy chain sequence set forth in SEQ ID NO:1 and/or a light chain sequence set forth in SEQ ID NO:2). For example, the antibody has the variable regions, VH and VL of cetuximab, and the CH and CL regions are different, such as CH and CL from a different IgG isotype or having one or more amino acid differences from the CH and CL regions of cetuximab. In some embodiments, the variant or derivative of cetuximab is one having the same CDRs as cetuximab and having a CH region with at least 90%, 92%, 95%, 98% or 99% identity to the CH region of cetuximab. In some embodiments, the variant or derivative of cetuximab is one having the same CDRs as cetuximab and having a CL region with at least 90%, 92%, 95%, 98% or 99% identity to the CL region of cetuximab. In some embodiments, the variant or derivative of cetuximab is one having the same CDRs as cetuximab and having a CH region with at least 90%, 92%, 95%, 98% or 99% identity with the CH and a CL region with at least 90%, 92%, 95%, 98% or 99% identity with the CL region of cetuximab. In some embodiments, the at least 90%, 92%, 95%, 98% or 99% identity with the CH and CL regions of cetuximab includes the positions of one or more lysine residues or exposed lysine residues in the CH and/or the CL regions.

In some embodiments, the variant or derivative of cetuximab is one having the same CDRs as cetuximab and having a CH region with 1, 2, 3, 4, 5, 6, 7, 8, 9, 10, 11, 12, 13, 14, 15, 16, 17, 18, 19, 20, or more than 20 amino acid substitutions compared to the CH region of cetuximab (e.g., comprising a heavy chain sequence set forth in SEQ ID NO:1 and/or a light chain sequence set forth in SEQ ID NO:2). In some embodiments, the variant or derivative of cetuximab is one having the same CDRs as cetuximab and having a CL region with 1, 2, 3, 4, 5, 6, 7, 8, 9, 10, or more than 10 amino acid substitutions compared to the CL region of cetuximab. In some embodiments, the variant or derivative of cetuximab is one having the same CDRs as cetuximab and having a CH region with 1, 2, 3, 4, 5, 6, 7, 8, 9, 10, 11, 12, 13, 14, 15, 16, 17, 18, 19, or 20 amino acid substitutions compared to the CH region of cetuximab, and a CL region with 1, 2, 3, 4, 5, 6, 7, 8, 9, or 10 amino acid substitutions compared to the CL region of cetuximab. In any of the provided embodiments, the one or more amino acid substitution can be at a position other than a lysine residue in the CH and/or the CL regions. In any of the provided embodiments, the one or more amino acid substitution can be at a position other than an exposed lysine residue in the CH and/or the CL regions. In some embodiments, the one or more amino acid substitution can include those disclosed in U.S. Pat. Nos. 6,737,056, 7,183,387, 7,632,497, 7,741,072, 7,960,512, 8,217,147, 8,388,955, 8,445,645, 8,652,466, 8,697,071, 8,735,547, 8,753,628, and 8,937,158, all of which are incorporated herein by reference.

In some embodiments, the antibody is a variant or derivative of cetuximab having a variant Fc region. In some embodiments, the variant Fc region comprises a variation, e.g., an amino acid residue substitution, compared to the Fc region of cetuximab (e.g., comprising a heavy chain sequence set forth in SEQ ID NO:1 and/or a light chain sequence set forth in SEQ ID NO:2). In some embodiments, the variation includes those disclosed in U.S. Pat. Nos. 6,737,056, 7,183,387, 7,632,497, 7,741,072, 7,960,512, 8,217,147, 8,388,955, 8,445,645, 8,652,466, 8,697,071, 8,735,547, 8,753,628, and 8,937,158, all of which are incorporated herein by reference for all purposes.

In the embodiments herein, a phthalocyanine dye, such as IR700, is conjugated to an antibody via individual reactive sites on the antibody. In some embodiments, the reactive sites are one or more lysine residues on the antibody on the light chain. In some embodiments, the reactive sites are one or more lysine residues on the antibody on the heavy chain. In some embodiments, the reactive sites are one or more lysine residues on the light chain of the antibody and are one or more lysine residues on the heavy chain of the antibody.

In some embodiments, at least one IR700 dye is conjugated to at least one lysine in the light chain of the antibody. In some embodiments, at least one IR700 dye is conjugated to at least one lysine in the light chain of the antibody and the conjugation position residues at a lysine in the constant region. In some embodiments, only one IR700 dye is conjugated to a lysine in the light chain of the antibody and the conjugation position residues at a lysine in the constant region. In some embodiments, the cetuximab-IR700 conjugate has at least one lysine in the light chain of a cetuximab at position 145 (K145) conjugated to an IR700 dye molecule (e.g., with reference to position numbering in the light chain sequence set forth in SEQ ID NO:2).

In some embodiments, at least one IR700 dye is conjugated to a lysine in the heavy chain of the antibody. In some embodiments, at least one IR700 dye is conjugated to a lysine in the heavy chain of the antibody and the conjugation position residues at a lysine in the constant region. In some embodiments, the cetuximab-IR700 conjugates is modified at one or more specific positions within the heavy chain of the cetuximab. In some embodiments, the cetuximab-IR700 conjugate has at least one lysine in the heavy chain of a cetuximab at one or more positions of K215, K292, K336, K416, and K449 conjugated to an IR700 dye molecule (e.g., with reference to position numbering in the heavy chain sequence set forth in SEQ ID NO:1). In some embodiments, the cetuximab-IR700 conjugate is modified at two or more specific positions within the heavy chain of the cetuximab and has at least one lysine in the heavy chain at positions of K215, K292, K336, K416, and K449 conjugated to an IR700 dye molecule. In some embodiments, the cetuximab-IR700 conjugate is modified at two or more specific positions within the heavy chain of the cetuximab and has at least two lysines in the heavy chain at positions of K215, K292, K336, K416, and K449 conjugated to an IR700 dye molecule. In some embodiments, the cetuximab-IR700 conjugate is modified at three or more specific positions within the heavy chain of the cetuximab and has 1, 2 or 3 of positions K215, K292, K336, K416, and K449 conjugated to an IR700 dye molecule.

### III. COMPOSITIONS OF CONJUGATES

Provided are compositions, such as pharmaceutical compositions, that contain one or more of the provided conjugates, e.g., cetuximab-IR700 conjugates. In some embodiments, a composition comprises a population or a plurality of conjugates, such as a population or a plurality of cetuximab-IR700 conjugates. Also provided are compositions that contain cetuximab-IR700 conjugates that, when digested with an endoprotease, such as trypsin, result in a population of certain peptides and modifications, such as conjugation of IR700 at a specific lysine positions or residues, and/or exhibit particular mass spectra (e.g., indicative of the presence of certain peptides and modifications) when analyzed or assessed by mass spectrometry, such as liquid chromatography/mass spectrometry (LC/MS). Also provided are compositions, such as analytical compositions, containing peptides and modified peptides (e.g., peptides containing amino acid residues conjugated with IR700) generated from digestion with an endoprotease, e.g., trypsin, of a composition comprising cetuximab-IR700 conjugates. In some aspects, such analytical compositions can be analyzed or assessed by mass spectrometry, such as LC/MS.

In some embodiments, the average number of dye molecule per antibody of the antibodies in the population can be from or from about 2 to at or about 5, such as from at or about 2 to at or about 4, e.g., about 3 or 3 or about 2.5 or 2.5. In some embodiments, the average number of dye molecules per light chain of the antibody in a population is at or about 0.4, 0.5, 0.6, 0.7, 0.8, 0.9, 1.0, 1.1, 1.2, 1.3, 1.4, 1.5 or about 1 or from at or about 0.2 to at or about 1.0, from at or about 0.2 to at or about 0.6 or from at or about 0.4 to at or about 0.5 or from at or about 0.5 to at or about 1.0. In some embodiments, the average number of dye molecule per heavy chain of the antibody in a population is at or about 1, about 2 or about 3 or about 4 or from at or about 1 to at or about 2 or from at or about 2 to at or about 3 or from at or about 2 to at or about 4 or about 2.5, 2.6, 2.7, 2.8, 2.9, 3.0, 3.1, 3.2, 3.3, 3.4 or 3.5.

In some of any of the embodiments provided herein, the lysine (K) residues or positions that are modified with or conjugated with IR700, are with reference to the amino acid position number in cetuximab. In some of any of the embodiments provided herein, the heavy chain lysine (K) residues or positions that are modified with or conjugated with IR700, are with reference to the amino acid position number in the heavy chain sequence set forth in SEQ ID NO:1. For example, K215, K292, K336, K416, or K449 of the heavy chain can refer to the lysines at positions 215, 292, 336, 416 or 449 of the heavy chain sequence set forth in SEQ ID NO:1. In some of any of the embodiments provided herein, the light chain lysine (K) residues or positions that are modified with or conjugated with IR700, are with reference to the amino acid position number in the light chain sequence set forth in SEQ ID NO:1. For example, K145 of the light chain can refer to the lysine at position 145 of the light chain sequence set forth in SEQ ID NO:2.

In some embodiments, a population comprises cetuximab-IR700 conjugates whereby a substantial percentage of the conjugates have at least one lysine residues of the light chain conjugated to IR700 dye. In some embodiments, at least at or about 10%, 15%, 20%, 25%, 30%, 35%, 40%, 45%, 50% or more than 50% of the antibodies in a population have at least one lysine (K) residues of the light chain conjugated to IR700 dye.

In some embodiments, a population comprises cetuximab-IR700 conjugates whereby a substantial percentage of the conjugates have at least position K145 of the light chain conjugated to IR700 dye. In some embodiments, at least at or about 10% of all cetuximab antibodies in a population are conjugated to IR700 at position K145 of the light chain. In some embodiments, at least at or about 10%, 15%, 20%, 30%, 35%, 40%, 45%, 50% or more than 50% of all conjugated antibodies in a population have IR700 at position K145 of the light chain (e.g., with reference to position numbering in the light chain sequence set forth in SEQ ID NO:2).

In some embodiments, a population comprises cetuximab antibodies whereby a substantial percentage of the IR700 conjugated cetuximab antibodies have at least position K215, K292, K336, K416, or K449 in the heavy chain conjugated to an IR700 dye molecule (e.g., with reference to position numbering in the heavy chain sequence set forth in SEQ ID NO:1). In some embodiments, at least at or about 10%, 15%, 20%, 30%, 35%, 40%, 45%, 50% or more than 50% of all cetuximab antibodies in a population are conjugated to IR700 at least one of position K215, K292, K336, K416, or K449 in the heavy chain. In some embodiments, at least 55%, 60%, 65% or 70% of all cetuximab conjugates in a population have IR700 at one or more of positions K215, K292, K336, K416, or K449 in the heavy chain. In some embodiments, at least at or about 10%, 15%, 20%, 30%, 35%, 40%, 45%, 50% or more than 50% of all cetuximab antibodies in a population have at least two IR700 dye molecule conjugated to the heavy chain, where one or two of the conjugated positions are K215, K292, K336, K416, or K449.

In some embodiments, the composition comprises a population of cetuximab-IR700 conjugates that can be digested by an endoprotease, such as trypsin, wherein the digested composition can be subsequently analyzed by liquid chromatography/mass spectrometry (LC/MS). In some embodiments, trypsin digestion of the composition provided herein yields a plurality of peptides, including peptides with amino acids (AA), monoisotopic masses ([MH]⁺) of unconjugated and IR700-conjugated peptides as set forth in Table 1:

TABLE 1
Cetuximab Light Chain

| AA | [MH]⁺ (Da) | | |
|---|---|---|---|
| (SEQ ID | Unconjugated | Peptide + | Conjugated |
| NO: 2) | Peptide | IR700 | Lysine |
| 46-61 | 1733.98 | 2503.18 | K49 |
| 104-108 | 658.42 | 1427.62 | K107 |
| 109-142 | 3724.9 | 4494.1 | K126 |
| 143-149 | 888.49 | 1657.69 | K145 |
| 146-169 | 2677.27 | 3446.47 | K149 |
| 184-190 | 890.44 | 1659.64 | K188 |
| 189-207 | 2141.08 | 2910.28 | K190 |
| 191-211 | 2380.17 | 3149.37 | K207 |

Cetuximab Heavy Chain

| AA | [MH]⁺ (Da) | | |
|---|---|---|---|
| (SEQ ID | Unconjugated | Peptide + | Conjugated |
| NO: 1) | Peptide | IR700 | Lysine |
| 1-38 | 4142.14 | 4911.34 | K5 |
| 39-66 | 3067.49 | 3836.69 | K43 |
| 72-81 | 1199.61 | 1968.81 | K75 |
| 76-97 | 4735.98 | 5505.18 | K81 |
| 124-149 | 2489.31 | 3258.51 | K135 |
| 213-216 | 517.31 | 1286.51 | K215 |
| 225-250 | 2844.46 | 3613.66 | K248 |
| 225-257 | 3660.87 | 4430.07 | K250 |
| 258-290 | 3797.81 | 4567.01 | K276 |
| 277-292 | 1906.94 | 2676.14 | K290 |
| 291-294 | 501.31 | 1270.51 | K292 |
| 304-322 | 2228.21 | 2997.41 | K319 |
| 325-336 | 1266.74 | 2035.94 | K328 |
| 329-340 | 1267.76 | 2036.96 | K336 |
| 358-372 | 1779.9 | 2549.1 | K362 |
| 373-411 | 4399.04 | 5168.24 | K394 |
| 412-418 | 818.47 | 1587.67 | K416 |
| 442-449 | 788.45 | 1557.65 | K449 |

In some embodiments, provided herein is a composition comprising a population or a plurality of cetuximab-IR700 conjugates, wherein trypsin digestion of the composition generates peptides that comprises a mixture of peptides some of which are conjugated to one or more IR700 molecules and other peptides that are not conjugated to IR700 molecules (unconjugated or unmodified peptides). In some embodiments, trypsin digestion of the provided composition contains peptides of the heavy chain of the cetuximab that contain an IR700 molecule conjugated to one or more of a lysine at position 5 (K5), K75, K215, K248, K292, K328, K336, K416, and/or K449 (e.g., with reference to position numbering in the heavy chain sequence set forth in SEQ ID NO:1). In some embodiments, trypsin digestion of the provided composition contains peptides of the light chain of the cetuximab that contain an IR700 molecule conjugated to one or more of a lysine at position 107 (K107), K145, K188, K190, and/or K207 (e.g., with reference to position numbering in the light chain sequence set forth in SEQ ID NO:2).

In some embodiments, the peptides conjugated to IR700 molecule(s) are detected by mass spectrometry (e.g., in positive ion mode). In some embodiments, trypsin digestion of the provided composition, generates modified and unmodified peptides that are detected by mass spectrometry.

Following liquid chromatography/mass spectrometry (LC/MS) procedures, extracted ion chromatograms (EICs) can be created by plotting the intensity of the signal observed at a selected mass-to-charge (m/z) value or series of selected m/z values in a series of mass spectra recorded as a function of retention time. The area under the curve of the EIC, determined by integration, for the selected values can be used to calculate the content of the selected component. To determine the percent conjugation for a selected peptide (having a selected mass-to-charge (m/z) value), the area under the curve corresponding to the selected peptide is divided by the sum of the areas under the curves for the conjugated peptide sequence and the unconjugated peptides (unmodified) that correspond to the same residues as the conjugated peptide, multiplied by 100 [percent conjugation=area of conjugated peptide/(area conjugated peptide+area of unmodified peptides)*100]. The selected peptide can be a singly charged peptide or a multiply charged peptide, such as a peptide having a charge (z) of 1, 2, 3, 4, 5, 6, 7, 8, 9, 10, or more.

In some embodiments, the integrated area of the extracted ion chromatogram (EIC) peaks corresponding to peptides of the heavy chain of cetuximab comprising an IR700 molecule conjugated to the lysine corresponding to position 5 (K5) of SEQ ID NO: 1 (percent conjugated peptide), is between at or about 2% and at or about 5% or between about 3% and at or about 5%, such as about 3%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or about 5% of the sum of the integrated area of the EIC peaks of the corresponding unmodified peptides and the EIC peaks corresponding the peptides modified at position K5. In some embodiments, the percent conjugated peptide at position K5 is about 3.8±1%. In some embodiments, the peptide conjugated to IR700 at position K5 and the unmodified peptide used to calculate the percent conjugated peptide correspond to amino acids 1-38 of SEQ ID NO: 1. In some embodiments, the peptide conjugated to IR700 at position K5 of the cetuximab heavy chain is an [MH₄]⁴⁺ peptide at about m/z 1243.09.

In some embodiments, the integrated area of the extracted ion chromatogram (EIC) peaks corresponding to peptides of the heavy chain of cetuximab comprising an IR700 molecule conjugated to the lysine corresponding to position 75 (K75) of SEQ ID NO: 1 (percent conjugated peptide), is between at or about 2% and at or about 5% or between at or about 3% and at or about 5%, such as about 3%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or about 5% of the sum of the integrated area of the EIC peaks of the corresponding unmodified peptides and the EIC peaks corresponding the peptides modified at position K75. In some embodiments, the percent conjugated peptide at position K75 is about 3.5±1%. In some embodiments, the peptides conjugated to IR700 at position K75 and the unmodified peptides used to calculate the percent conjugated peptide correspond to amino acids 72-81 of SEQ ID NO: 1. In some embodiments, the peptide conjugated to IR700 at position K75 of the cetuximab heavy chain is an [MH₂]²⁺ peptide at about m/z 984.91.

In some embodiments, the integrated area of the extracted ion chromatogram (EIC) peaks corresponding to peptides of the heavy chain of cetuximab comprising an IR700 molecule conjugated to the lysine corresponding to position 215 (1(215) of SEQ ID NO: 1 (percent conjugated peptide), is between at or about 8% and at or about 11% or between at or about 9% and at or about 11%, such as about 9%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, or about 11% of the sum of the integrated area of the EIC peaks of the corresponding unmodified peptides and the EIC peaks corresponding the peptides modified at position K215. In some embodiments, the percent conjugated peptide at position K215 is about 10.0±1%. In some embodiments, the peptides conjugated to IR700 at position K215 and the unmodified peptides used to calculate the percent conjugated peptide correspond to amino acids 213-216 of SEQ ID NO: 1. In some embodiments, the peptide conjugated to IR700 at position K215 of the cetuximab heavy chain is an [MH₂]²⁺ peptide at about m/z 643.76.

In some embodiments, the integrated area of the extracted ion chromatogram (EIC) peaks corresponding to peptides of the heavy chain of cetuximab comprising an IR700 molecule conjugated to the lysine corresponding to position 248 (K248) of SEQ ID NO: 1 (percent conjugated peptide), is between at or about 0.5% and at or about 2.5%, such as about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or about 2.5% of the sum of the integrated area of the EIC peaks of the corresponding unmodified peptides and the EIC peaks corresponding the peptides modified at position K248. In some embodiments, the percent conjugated peptide at position K248 is about 1.7±1%. In some embodiments, the peptides conjugated to IR700 at position K248 and the unmodified peptides used to calculate the percent conjugated peptide correspond to amino acids 225-250 of SEQ ID NO: 1. In some embodiments, the peptide conjugated to IR700 at position K248 of the cetuximab heavy chain is an [MH₃]³⁺ peptide at about m/z 1205.22.

In some embodiments, the integrated area of the extracted ion chromatogram (EIC) peaks corresponding to peptides of the heavy chain of cetuximab comprising an IR700 molecule conjugated to the lysine corresponding to position 292 (K292) of SEQ ID NO: 1 (percent conjugated peptide), is between at or about 8% and at or about 12%, such as about 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, or about 12% of the sum of the integrated area of the EIC peaks of the corresponding unmodified peptides and the EIC peaks corresponding the peptides modified at position K292. In some embodiments, the percent conjugated peptide at position K292 is about 10.2±1%. In some embodiments, the peptides conjugated to IR700 at position K292 and the unmodified peptides used to calculate the percent conjugated peptide correspond to amino acids 291-294 of SEQ ID NO: 1. In some embodiments, the peptide conjugated to IR700 at position K292 of the cetuximab heavy chain is an [MH₃]³⁺ peptide at about m/z 424.16.

In some embodiments, the integrated area of the extracted ion chromatogram (EIC) peaks corresponding to peptides of the heavy chain of cetuximab comprising an IR700 molecule conjugated to the lysine corresponding to position 328 (K328) of SEQ ID NO: 1 (percent conjugated peptide), is between at or about 0.2% and at or about 2.5%, such as about 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, or about 2.5% of the sum of the integrated area of the EIC peaks of the corresponding unmodified peptides and the EIC peaks corresponding the peptides modified at position K328. In some embodiments, the percent conjugated peptide at position K328 is about 1.3±1%. In some embodiments, the peptides conjugated to IR700 at position K328 and the unmodified peptides used to calculate the percent conjugated peptide correspond to amino acids 325-336 of SEQ ID NO: 1. In some embodiments, the peptide conjugated to IR700 at position K328 of the cetuximab heavy chain is an [MH₂]²⁺ peptide at about m/z 1018.47.

In some embodiments, the integrated area of the extracted ion chromatogram (EIC) peaks corresponding to peptides of the heavy chain of cetuximab comprising an IR700 molecule conjugated to the lysine corresponding to position 336 (K336) of SEQ ID NO: 1 (percent conjugated peptide), is between at or about 4.5% and at or about 7%, such as about 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, or about 7.0% of the sum of the integrated area of the EIC peaks of the corresponding unmodified peptides and the EIC peaks corresponding the peptides modified at position K336. In some embodiments, the percent conjugated peptide at position K336 is about 5.9±1%. In some embodiments, the peptides conjugated to IR700 at position K336 and the unmodified peptides used to calculate the percent conjugated peptide correspond to amino acids 329-340 of SEQ ID NO: 1. In some embodiments, the peptide conjugated to IR700 at position K336 of the cetuximab heavy chain is an [MH₂]²⁺ peptide at about m/z 1018.98.

In some embodiments, the integrated area of the extracted ion chromatogram (EIC) peaks corresponding to peptides of the heavy chain of cetuximab comprising an IR700 molecule conjugated to the lysine corresponding to position 416 (K416) of SEQ ID NO: 1 (percent conjugated peptide), is between at or about 9.5% and at or about 13%, such as about 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, or about 13.0% of the sum of the integrated area of the EIC peaks of the corresponding unmodified peptides and the EIC peaks corresponding the peptides modified at position K416. In some embodiments, the percent conjugated peptide at position K416 is about 11.2±1%. In some embodiments, the peptides conjugated to IR700 at position K416 and the unmodified peptides used to calculate the percent conjugated peptide correspond to amino acids 412-418 of SEQ ID NO: 1. In some embodiments, the peptide conjugated to IR700 at position K416 of the cetuximab heavy chain is an [MH₃]³⁺ peptide at about m/z 529.89.

In some embodiments, the integrated area of the extracted ion chromatogram (EIC) peaks corresponding to peptides of the heavy chain of cetuximab comprising an IR700 molecule conjugated to the lysine corresponding to position 449 (K449) of SEQ ID NO: 1 (percent conjugated peptide), is between at or about 6% and at or about 10%, such as about 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or about 10.0% of the sum of the integrated area of the EIC peaks of the corresponding unmodified peptides and the EIC peaks corresponding the peptides modified at position K449. In some embodiments, the percent conjugated peptide at position K449 is about 7.6±1%. In some embodiments, the peptides conjugated to IR700 at position K449 and the unmodified peptides used to calculate the percent conjugated peptide correspond to amino acids 442-449 of SEQ ID NO: 1. In some embodiments, the peptide conjugated to IR700 at position K449 of the cetuximab heavy chain is an [MH₂]²⁺ peptide at about m/z 779.33.

In some embodiments, the integrated area of the extracted ion chromatogram (EIC) peaks corresponding to peptides of the light chain of cetuximab comprising an IR700 molecule conjugated to the lysine corresponding to position 107 (K107) of SEQ ID NO: 2 (percent conjugated peptide), is between at or about 2% and at or about 5%, such as about 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or about 5.0% of the sum of the integrated area of the EIC peaks of the corresponding unmodified peptides and the EIC peaks corresponding the peptides modified at position K107. In some embodiments, the percent conjugated peptide at position K107 is about 3.4±1%. In some embodiments, the peptides conjugated to IR700 at position K107 and the unmodified peptides used to calculate the percent conjugated peptide correspond to amino acids 104-108 of SEQ ID NO: 2. In some embodiments, the peptide conjugated to IR700 at position K107 of the cetuximab light chain is an [IVII-12]²⁺ peptide at about m/z 714.34.

In some embodiments, the integrated area of the extracted ion chromatogram (EIC) peaks corresponding to peptides of the light chain of cetuximab comprising an IR700 molecule conjugated to the lysine corresponding to position 145 (K145) of SEQ ID NO: 2 (percent conjugated peptide), is between at or about 7% and at or about 11%, such as about 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, or about 11.0% of the sum of the integrated area of the EIC peaks of the corresponding unmodified peptides and the EIC peaks corresponding the peptides modified at position K145. In some embodiments, the percent conjugated peptide at position K145 is about 9.3±1%. In some embodiments, the peptides conjugated to IR700 at position K145 and the unmodified peptides used to calculate the percent conjugated peptide correspond to amino acids 143-149 of SEQ ID NO: 2. In some embodiments, the peptide conjugated to IR700 at position K145 of the cetuximab light chain is an [MH₂]²⁺ peptide at about m/z 829.36.

In some embodiments, the integrated area of the extracted ion chromatogram (EIC) peaks corresponding to peptides of the light chain of cetuximab comprising an IR700 molecule conjugated to the lysine corresponding to position 188 (K188) of SEQ ID NO: 2 (percent conjugated peptide), is between at or about 0.5% and at or about 4%, such as about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or about 4.0% of the sum of the integrated area of the EIC peaks of the corresponding unmodified peptides and the EIC peaks corresponding the peptides modified at position K188. In some embodiments, the percent conjugated peptide at position K188 is about 2.1±1%. In some embodiments, the peptides conjugated to IR700 at position K188 and the unmodified peptides used to calculate the percent conjugated peptide correspond to amino acids 184-190 of SEQ ID NO: 2. In some embodiments, the peptide conjugated to IR700 at position K188 of the cetuximab light chain is an [MH₄]⁴⁺ peptide at about m/z 415.67.

In some embodiments, the integrated area of the extracted ion chromatogram (EIC) peaks corresponding to peptides of the light chain of cetuximab comprising an IR700 molecule conjugated to the lysine corresponding to position 190 (K190) of SEQ ID NO: 2 (percent conjugated peptide), is between at or about 1.5% and at or about 5%, such as about 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or about 5.0% of the sum of the integrated area of the EIC peaks of the corresponding unmodified peptides and the EIC peaks corresponding the peptides modified at position K190. In some embodiments, the percent conjugated peptide at position K190 is about 3.5±1%. In some embodiments, the peptides conjugated to IR700 at position K190 and the unmodified peptides used to calculate the percent conjugated peptide correspond to amino acids 189-207 of SEQ ID NO: 2. In some embodiments, the peptide conjugated to IR700 at position K190 of the cetuximab light chain is an [MH₃]³⁺ peptide at about m/z 970.76.

In some embodiments, the integrated area of the extracted ion chromatogram (EIC) peaks corresponding to peptides of the light chain of cetuximab comprising an IR700 molecule conjugated to the lysine corresponding to position 207 (1(207) of SEQ ID NO: 2 (percent conjugated peptide), is between at or about 0.5% and at or about 4%, such as about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, or about 4.0% of the sum of the integrated area of the EIC peaks of the corresponding unmodified peptides and the EIC peaks corresponding the peptides modified at position K207. In some embodiments, the percent conjugated peptide at position K207 is about 2.0±1%. In some embodiments, the peptides conjugated to IR700 at position K207 and the unmodified peptides used to calculate the percent conjugated peptide correspond to amino acids 191-211 of SEQ ID NO: 2. In some embodiments, the peptide conjugated to IR700 at position K207 of the cetuximab light chain is an [MH₃]³⁺ peptide at about m/z 1050.48.

In some embodiments, trypsin digestion of the provided composition generates a pattern of calculated fractions of the plurality of IR700-conjugated peptides (% conjugated peptide) as detected by mass spectrometry (e.g., in positive ion mode), generating a peptide mass fingerprint of the composition.

In some embodiments, the trypsin-digested, LC/MS-analyzed composition contains a population of peptides where the abundance of a first set of detected IR700-modified peptides in the composition (or the frequency of their detection) is greater than the abundance or frequency of detection of other modified peptides and the first set includes one or more of peptides of the heavy chain of cetuximab comprising an IR700 molecule conjugated to the lysine corresponding to position 215 (K215) of SEQ ID NO: 1; peptides of the heavy chain of cetuximab comprising an IR700 molecule conjugated to the lysine corresponding to position 292 (K292) of SEQ ID NO: 1; peptides of the heavy chain of cetuximab comprising an IR700 molecule conjugated to the lysine corresponding to position 416 (K416) of SEQ ID NO: 1; and peptides of the light chain of cetuximab comprising an IR700 molecule conjugated to the lysine corresponding to position 145 (K145) of SEQ ID NO: 2. In some of such embodiments, the percent area of the IR700-conjugated EIC peak is at least about 7.5% of the total area of EIC peaks of the corresponding modified and unmodified peptides, such as about at least 8%, 8.5% or at least 9% of the total area of EIC peaks of the corresponding modified and unmodified peptides. In some embodiments, the percent area of the IR700-conjugated EIC peak is between about 7.5% and 20% of the total area of EIC peaks of the corresponding modified and unmodified peptides, such as about 7.5%, 8%, 8.5%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 12%, 12.5%, 13%, 13.5%, 14%, 14.5%, 15%, 15.5%, 16%, 16.5%, 17%, 18%, 19%, or at least 20% of the total area of EIC peaks of the corresponding modified and unmodified peptides.

In some embodiments, the abundance of a second set of detected IR700-modified peptides in the composition (or the frequency of their detection) is greater than the abundance or frequency of detection of other modified peptides (but less in abundance or frequency of detection than the first set), and such second set includes one or more of peptides of the heavy chain of cetuximab comprising an IR700 molecule conjugated to the lysine corresponding to position 336 (K336) of SEQ ID NO: 1; and peptides of the heavy chain of cetuximab comprising an IR700 molecule conjugated to the lysine corresponding to position 449 (K449) of SEQ ID NO: 1. In some of such embodiments, the percent area of the IR700-conjugated EIC peak is at least about 4% of the total area of EIC peaks of the corresponding modified and unmodified peptides, such as about at least 4%, 4.5% or at least 5% of the total area of EIC peaks of the corresponding modified and unmodified peptides, but less abundant or frequent than the first set of modified peptides. In some embodiments, the percent area of the IR700-conjugated EIC peak is between about 4% and 9% of the total area of EIC peaks of the corresponding modified and unmodified peptides, such as about 4%, 4.5%, 5%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.5%, or 9% of the total area of EIC peaks of the corresponding modified and unmodified peptides.

In some embodiments, the abundance of a third set of detected IR700-modified peptides in the composition (or the frequency of their detection) is greater than the abundance or frequency of detection of other modified peptides (but less in abundance or frequency of detection than the first set and second set), and such third set includes one or more peptides of the light chain of cetuximab comprising an IR700 molecule conjugated to the lysine corresponding to position 107 (K107) of SEQ ID NO: 2; peptides of the light chain of cetuximab comprising an IR700 molecule conjugated to the lysine corresponding to position 190 (K190) of SEQ ID NO: 2. peptides of the heavy chain of cetuximab comprising an IR700 molecule conjugated to the lysine corresponding to position 5 (K5) of SEQ ID NO: 1; and peptides of the heavy chain of cetuximab comprising an IR700 molecule conjugated to the lysine corresponding to position 75 (1(75) of SEQ ID NO: 1. In some of such embodiments, the percent area of the IR700-conjugated EIC peak is at least about 2% of the total area of EIC peaks of the corresponding modified and unmodified peptides, such as about at least 2.5%, 2.7% or at least 3% of the total area of EIC peaks of the corresponding modified and unmodified peptides, but less abundant or frequent than the first set and second set of modified peptides. In some embodiments, the percent area of the IR700-conjugated EIC peak is between about 2.5% and 5.5% of the total area of EIC peaks of the corresponding modified and unmodified peptides, such as about 2.5%, 2.7%, 3%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or 5.5% of the total area of EIC peaks of the corresponding modified and unmodified peptides.

In some embodiments, the abundance of a fourth set of detected IR700-modified peptides in the composition (or the frequency of their detection) is greater than the abundance or frequency of detection of other modified peptides (but less in abundance or frequency of detection than the first set, second set, and third set), and such fourth set includes one or more peptides of the heavy chain of cetuximab comprising an IR700 molecule conjugated to the lysine corresponding to position 248 (K248) of SEQ ID NO: 1; peptides of the heavy chain of cetuximab comprising an IR700 molecule conjugated to the lysine corresponding to position 328 (K328) of SEQ ID NO: 1; peptides of the light chain of cetuximab comprising an IR700 molecule conjugated to the lysine corresponding to position 188 (K188) of SEQ ID NO: 2; and peptides of the light chain of cetuximab comprising an IR700 molecule conjugated to the lysine corresponding to position 207 (K207) of SEQ ID NO: 2. In some of such embodiments, the percent area of the IR700-conjugated EIC peak is at least about 0.5% of the total area of EIC peaks of the corresponding modified and unmodified peptides, such as about at least 1%, 1.1% or at least 1.2% of the total area of EIC peaks of the corresponding modified and unmodified peptides, but less abundant or frequent than the first set, second set, and third set of modified peptides. In some embodiments, the percent area of the IR700-conjugated EIC peak is between about 0.5% and 3% of the total area of EIC peaks of the corresponding modified and unmodified peptides, such as about 0.5%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3% of the total area of EIC peaks of the corresponding modified and unmodified peptides.

In some embodiments, the trypsin-digested, LC/MS-analyzed composition provided herein contains peptides with IR700 conjugated to a lysine corresponding to position 5 (K5) of SEQ ID NO: 1, wherein the percent area of the conjugated EIC peak is at least about 2.5% of the total area of EIC peaks of the corresponding modified and unmodified peptide; peptides comprising an IR700 molecule conjugated to a lysine corresponding to position 75 (K75) of SEQ ID NO: 1, wherein the percent area of the conjugated EIC peak is at least about 2.5% of the total area of EIC peaks of the corresponding modified and unmodified polypeptide; peptides comprising an IR700 molecule conjugated to a lysine corresponding to position 215 (K215) of SEQ ID NO: 1, wherein the percent area of the conjugated EIC peak is at least about 9%, of the total area of EIC peaks of the corresponding modified and unmodified polypeptide; peptides comprising an IR700 molecule conjugated to a lysine corresponding to position 248 (K248) of SEQ ID NO: 1, wherein the percent area of the conjugated EIC peak is at least about 0.5% of the total area of EIC peaks of the corresponding modified and unmodified polypeptide; peptides comprising an IR700 molecule conjugated to a lysine corresponding to position 292 (K292) of SEQ ID NO: 1, wherein the percent area of the conjugated EIC peak is about at least about 8.5%, of the total area of EIC peaks of the corresponding modified and unmodified polypeptide; peptides comprising an IR700 molecule conjugated to a lysine corresponding to position 328 (K328) of SEQ ID NO: 1, wherein the percent area of the conjugated EIC peak is at least about 0.5% of the total area of EIC peaks of the corresponding modified and unmodified polypeptide; peptides comprising an IR700 molecule conjugated to a lysine corresponding to position 336 (K336) of SEQ ID NO: 1, wherein the percent area of the conjugated EIC peak is at least about 4.5% of the total area of EIC peaks of the corresponding modified and unmodified polypeptide; peptides comprising an IR700 molecule conjugated to a lysine corresponding to position 416 (K416) of SEQ ID NO: 1, wherein the percent area of the conjugated EIC peak is at least about 9%, of the total area of EIC peaks of the corresponding modified and unmodified polypeptide; peptides comprising an IR700 molecule conjugated to a lysine corresponding to position 449 (K449) of SEQ ID NO: 1, wherein the percent area of the conjugated EIC peak is at least about 7% of the total area of EIC peaks of the corresponding modified and unmodified polypeptide; peptides comprising an IR700 molecule conjugated to a lysine corresponding to position 107 (K107) of SEQ ID NO: 2, wherein the percent area of the conjugated EIC peak is at least about 2.5% of the total area of EIC peaks of the corresponding modified and unmodified polypeptide; peptides comprising an IR700 molecule conjugated to a lysine corresponding to position 145 (K145) of SEQ ID NO: 1, wherein the percent area of the conjugated EIC peak is at least about 8.5%, of the total area of EIC peaks of the corresponding modified and unmodified polypeptide; peptides comprising an IR700 molecule conjugated to a lysine corresponding to position 188 (K188) of SEQ ID NO: 2, wherein the percent area of the conjugated EIC peak is at least about 1% of the total area of EIC peaks of the corresponding modified and unmodified polypeptide; peptides comprising an IR700 molecule conjugated to a lysine corresponding to position 190 (K190) of SEQ ID NO: 2, wherein the percent area of the conjugated EIC peak is at least about 2.5% of the total area of EIC peaks of the corresponding modified and unmodified polypeptide; and peptides comprising an IR700 molecule conjugated to a lysine corresponding to position 207 (K207) of SEQ ID NO: 2, wherein the percent area of the conjugated EIC peak is at least about 1% of the total area of EIC peaks of the corresponding modified and unmodified polypeptide.

In some embodiments, the percent area of the conjugated EIC peak is about 3.8±1% for the peptides comprising an IR700 molecule conjugated to a lysine corresponding to position 5 (K5) of SEQ ID NO: 1; the percent area of the conjugated EIC peak is about 3.5±1% for the peptides comprising an IR700 molecule conjugated to a lysine corresponding to position 75 (K75) of SEQ ID NO: 1; the percent area of the conjugated EIC peak is about 10.0±1%, for the peptides comprising an IR700 molecule conjugated to a lysine corresponding to position 215 (K215) of SEQ ID NO: 1; the percent area of the conjugated EIC peak is about 1.7±1% for the peptides comprising an IR700 molecule conjugated to a lysine corresponding to position 248 (K248) of SEQ ID NO: 1; the percent area of the conjugated EIC peak is about 10.2±1% for the peptides comprising an IR700 molecule conjugated to a lysine corresponding to position 292 (K292) of SEQ ID NO: 1; the percent area of the conjugated EIC peak is about 1.3±1% for the peptides comprising an IR700 molecule conjugated to a lysine corresponding to position 328 (K328) of SEQ ID NO: 1; the percent area of the conjugated EIC peak about 5.9±1% for the peptides comprising an IR700 molecule conjugated to a lysine corresponding to position 336 (K336) of SEQ ID NO: 1; the percent area of the conjugated EIC peak is about 11.2±1%, for the peptides comprising an IR700 molecule conjugated to a lysine corresponding to position 416 (K416) of SEQ ID NO: 1; the percent area of the conjugated EIC peak about 7.6±1% for the peptides comprising an IR700 molecule conjugated to a lysine corresponding to position 449 (K449) of SEQ ID NO: 1; the percent area of the conjugated EIC peak is about 3.4±1% for the peptides comprising an IR700 molecule conjugated to a lysine corresponding to position 107 (K107) of SEQ ID NO: 2; the percent area of the conjugated EIC peak is about 9.3±1%, for the peptides comprising an IR700 molecule conjugated to a lysine corresponding to position 145 (K145) of SEQ ID NO: 2; the percent area of the conjugated EIC peak is about 2.1±1% for the peptides comprising an IR700 molecule conjugated to a lysine corresponding to position 188 (K188) of SEQ ID NO: 2; the percent area of the conjugated EIC peak is about 3.5±1% for the peptides comprising an IR700 molecule conjugated to a lysine corresponding to position 190 (K190) of SEQ ID NO: 2; and the percent area of the conjugated EIC peak is about 2±1% for the peptides comprising an IR700 molecule conjugated to a lysine corresponding to position 207 (K207) of SEQ ID NO: 2.

In some embodiments, the percent area is calculated using peptides having the amino sequences corresponding to the sequences of amino acids corresponding to amino acids 1-38, amino acids 72-81 amino acids 213-216, amino acids 225-250, amino acids 291-294, amino acids 325-336, amino acids 329-340, amino acids 412-418, and amino acids 442-449 of SEQ ID NO: 1; and the sequences of amino acids corresponding to amino acids 104-108, amino acids 143-149, amino acids 184-190, amino acids 189-207, and amino acids 191-211 of SEQ ID NO: 2.

In some embodiments, provided herein is a composition comprising a population of cetuximab molecules conjugated to IR700 molecules, wherein when the composition is analyzed by mass spectrometry: the ratio between peptides that contain IR700 dye conjugation at light chain lysine 145 (K145) and peptides that contain IR700 dye conjugation at heavy chain lysine 215 (K215) is about 2:1 to about 1:2, optionally about 1:1; the ratio between peptides that contain IR700 dye conjugation at light chain lysine 145 (K145) and peptides that contain IR700 dye conjugation at heavy chain lysine 292 (K292) is about 2:1 to about 1:2, optionally about 1:1; the ratio between peptides that contain IR700 dye conjugation at light chain lysine 145 (K145) and peptides that contain IR700 dye conjugation at heavy chain lysine 336 (K336) is about 2:1 to about 1:2, optionally about 1:1; the ratio between peptides that contain IR700 dye conjugation at light chain lysine 145 (K145) and peptides that contain IR700 dye conjugation at heavy chain lysine 416 (K416) is about 2:1 to about 1:2, optionally about 1:1; and/or the ratio between peptides that contain IR700 dye conjugation at light chain lysine 145 (K145) and peptides that contain IR700 dye conjugation at heavy chain lysine 449 (K449) is about 2:1 to about 1:2, optionally about 1:1.

In some embodiments, provided herein is a composition comprising a population of cetuximab molecules, wherein when the composition is analyzed by mass spectrometry: the ratio between peptides that contain IR700 dye conjugation at heavy chain lysine 215 (K215) and peptides that contain IR700 dye conjugation at heavy chain lysine 292 (K292) is about 2:1 to about 1:2, optionally about 1:1; the ratio between peptides that contain IR700 dye conjugation at heavy chain lysine 215 (K215) and peptides that contain IR700 dye conjugation at heavy chain lysine 336 (K336) is about 2:1 to about 1:2, optionally about 1:1; the ratio between peptides that contain IR700 dye conjugation at heavy chain lysine 215 (K215) and peptides that contain IR700 dye conjugation at heavy chain lysine 416 (K416) is about 2:1 to about 1:2, optionally about 1:1; and/or the ratio between peptides that contain IR700 dye conjugation at heavy chain lysine 215 (K215) and peptides that contain IR700 dye conjugation at heavy chain lysine 449 (K449) is about 2:1 to about 1:2, optionally about 1:1.

In some embodiments, provided herein is a composition comprising a population of cetuximab molecules, wherein when the composition is analyzed by mass spectrometry: the ratio between peptides that contain IR700 dye conjugation at heavy chain lysine 292 (K292) and peptides that contain IR700 dye conjugation at heavy chain lysine 336 (K336) is about 2:1 to about 1:2, optionally about 1:1; the ratio between peptides that contain IR700 dye conjugation at heavy chain lysine 292 (K292) and peptides that contain IR700 dye conjugation at heavy chain lysine 416 (K416) is about 2:1 to about 1:2, optionally about 1:1; and/or the ratio between peptides that contain IR700 dye conjugation at heavy chain lysine 292 (K292) and peptides that contain IR700 dye conjugation at heavy chain lysine 449 (K449) is about 2:1 to about 1:2, optionally about 1:1.

In some embodiments, provided herein is a composition comprising a population of cetuximab molecules, wherein when the composition is analyzed by mass spectrometry: the ratio between peptides that contain IR700 dye conjugation at heavy chain lysine 336 (K336) and peptides that contain IR700 dye conjugation at heavy chain lysine 416 (K416) is about 2:1 to about 1:2, optionally about 1:1; and/or the ratio between peptides that contain IR700 dye conjugation at heavy chain lysine 336 (K336) and peptides that contain IR700 dye conjugation at heavy chain lysine 449 (K449) is about 2:1 to about 1:2, optionally about 1:1.

In some embodiments, provided herein is a composition comprising a population of cetuximab molecules, wherein when the composition is analyzed by mass spectrometry the ratio between peptides that contain IR700 dye conjugation at heavy chain lysine 416 (K416) and peptides that contain IR700 dye conjugation at heavy chain lysine 449 (K449) is about 2:1 to about 1:2, optionally about 1:1.

In some embodiments, provided herein is a composition comprising a population of cetuximab molecules, wherein when the composition is analyzed by mass spectrometry, the ratio among peptides that contain IR700 dye conjugation at light chain lysine 145 (K145), peptides that contain IR700 dye conjugation at heavy chain lysine 215 (K215), peptides that contain IR700 dye conjugation at heavy chain lysine 292 (K292), and peptides that contain IR700 dye conjugation at heavy chain lysine 416 (1(416) is about 1:1:1:1.

In some embodiments, provided herein is a composition comprising SEQ ID NO: 1, wherein lysine 145 is conjugated to an IR700 dye. In some aspects, the composition further comprises SEQ ID NO: 2, wherein lysine 215, lysine 292, and lysine 416 of SEQ ID NO: 2 and optionally lysine 449 of SEQ ID NO: 2 are each conjugated to an IR700 dye.

In some embodiments, provided herein is a conjugate comprising a cetuximab conjugated to IR700 dye, wherein at least one IR700 dye is conjugated to a lysine in the light chain of the antibody. In some aspects, the IR700 dye is conjugated at a lysine at position 145 of the light chain of the cetuximab.

In some embodiments, provided herein is a conjugate comprising a cetuximab conjugated to IR700 dye, wherein at least one IR700 dye is conjugated to a lysine in the heavy chain of the antibody. In some aspects, the IR700 dye molecule is conjugated at a lysine residues in the heavy chain selected from the group consisting of K215, K292, K416 and K449.

In any of the provided embodiments, the cetuximab can comprise IR700 dye conjugated at 2 or more lysine residues in the heavy chain. In some aspects, the cetuximab comprises IR700 dye conjugated at 3 lysine residues in the heavy chain.

In any of the provided embodiments, the cetuximab can have IR700 dye conjugated to the light chain of the cetuximab at lysine residue 145.

In any of the provided embodiments, the conjugate may be activated by irradiation with a wavelength between about 600 nm and 850 nm and thereby acquires cell killing activity. In some aspects, the wavelength is 690 nm±50 nm.

In some embodiments, provided herein is a composition comprising a population of cetuximab conjugated to IR700 dye, wherein at least 70%, 80%, 90% or more than 90% of the population has at least one IR700 dye conjugated to a lysine on the light chain of cetuximab. In some aspects, the predominant lysine residue conjugated in the light chain is K145.

In some embodiments, provided herein is a composition comprising a population of cetuximab conjugated to IR700 dye, wherein at least 70%, 80%, 90% or more than 90% of the population has at least one IR700 dye conjugated to a lysine on the heavy chain of cetuximab. In some aspects, the predominant lysine residue conjugated in the heavy chain is one or more of K215, K292, K416 and K449.

In any of the provided embodiments, at least 70%, 80%, 90% or more than 90% of the population of cetuximab molecules can have IR700 dye conjugated to two or more lysines on the heavy chain of cetuximab.

In some embodiments, provided herein is a composition comprising a population of cetuximab conjugated to IR700 dye, wherein no more than about 20% of the population is unconjugated antibody. In some aspects, less than 10% of the population is unconjugated antibody. In some aspects, less than about 10%, 9%, 8%, 7%, 6%, 5%, 4%, 3%, 2% or 1% of the population is unconjugated antibody.

In any of the provided embodiments, the ratio of dye to cetuximab in the population can be about 2:1, about 2.5:1 or about 3:1.

In some embodiments, provided herein is a composition comprising cetuximab conjugated to IR700 dye, wherein the composition comprises less than or less than about 0.6%, 0.5%, 0.4% or 0.3% free dye (e.g., unconjugated IR700 dye). In some aspects, the composition comprises at least or at least about 95%, 96% 97% or 98% monomer. In some aspects, the composition comprises less than or less than about 5%, 4% or 3% high molecular weight species. In some aspects, the composition comprises less than or less than about 30%, 20%, 25%, 20%, 15% or 10% unconjugated antibody. In some aspects, the percentage of free dye (e.g., unconjugated IR700 dye) is substantially unchanged after storage of the composition for 6 months in dark or reduced light conditions.

In any of the provided embodiments, at least 70%, 80%, 90% or more than 90% of the population can have at least one IR700 dye conjugated to a lysine on the light chain of the antibody. In some aspects, the predominant lysine residue conjugated in the light chain is K145.

In any of the provided embodiments, at least 70%, 80%, 90% or more than 90% of the population can have at least one IR700 dye conjugated to a lysine on the heavy chain of the antibody. In some aspects, the predominant lysine residue conjugated in the heavy chain is one or more of K215, K292, K416 and K449.

### IV. METHODS OF MANUFACTURE OF CETUXIMAB-IR700 CONJUGATES

Also provided herein are methods of making, manufacturing or generating the conjugates and/or compositions provided herein, which include a cetuximab-IR700 conjugate and populations of cetuximab-IR700 conjugates having IR700 dye at one or more specific lysine residues or positions on the light chain and/or the heavy chain. In some embodiments, the method includes providing conditions such that a specific dye to antibody ratio is achieved. In some embodiments, the method includes providing conditions such that following digestion and analysis of the composition comprising the conjugates, specific types and/or relative proportion of peptides and modified peptides and/or mass spectra (e.g., as assessed by LC/MS) are achieved. In some embodiments, the method includes providing conditions such that the amount of dye associated with the cetuximab at positions other than a lysine position is substantially reduced such the substantial majority of IR700 dye molecules in the composition are covalently conjugated at a lysine within the cetuximab.

In some embodiments, provided is a cetuximab-IR700 conjugate and compositions comprising populations of the cetuximab-IR700 conjugates that are stable and consistent. With reference to the conjugate, the term “consistent” refers to a composition of a conjugate or population of conjugates in which, after the conjugation process, the DAR (dye to antibody ratio) of the conjugate and the amount of free dye in the composition remain substantially the same over time. In some processes, the IR700 dye may become associated with the antibody at positions other than at a lysine, such as through thiol esters and tyrosine esters. These non-lysine conjugation positions are unstable, so that over time, IR700 dye becomes dissociated and is released into the composition, such that the composition is not consistent over time.

Provided herein are methods of manufacturing consistent cetuximab-IR700 conjugates and consistent populations of cetuximab-IR700 conjugates. In some or all of the manufacturing steps described herein, the reaction components and the reaction steps are performed under light-protected conditions, such that the dye and/or the conjugate are not exposed to any environmental light or are not exposed to light with an intensity greater than 700 lux, greater than 600 lux, greater than 500 lux, greater than 400 lux, greater than 300 lux, greater than 200 lux, or greater than 100 lux. In some embodiments of the manufacturing, the dye and the conjugate are not exposed to light with an intensity greater than 700 lux for more than 10 minutes or more than 5 minutes. In some embodiments of the manufacturing, the dye and the conjugate are not exposed to light with an intensity greater than 200 lux for more than 10 minutes or more than 5 minutes. See, e.g., PCT/US2016/047636, published as WO2017/031363.

In some embodiments, the methods of manufacturing a conjugate include a step of preparing or producing the conjugate. In some embodiments, such methods include providing a phthalocyanine dye. In some embodiments, the phthalocyanine dye is provided in an aqueous form, such as an aqueous solution. In some embodiments, the dye is provided in a lyophilized form, such as a lyophilized powder, and is reconstituted or dissolved into a solvent to form an aqueous solution. For example, in some embodiments, the phthalocyanine dye containing the reactive group, e.g., IR700 NHS ester, is dissolved in a solvent. In some embodiments, the methods include a step of dissolving the phthalocyanine dye in a solvent, such as prior to conjugation of the dye to antibody. In some embodiments, the solvent is an organic solvent, such as dimethyl sulfoxide (DMSO) or DMF. In some examples, the solvent is a water-based solvent. In some embodiments, the dye is dissolved in solvent to a concentration in a range from at or about 0.1 mg/mL to at or about 100 mg/ml, from at or about 1 mg/mL to at or about 50 mg/mL, from at or about 1 mg/mL to at or about 15 mg/mL, or is dissolved in solvent to a concentration of or of about 10 mg/mL. In some embodiments, during the steps of preparing the dye for use in the method, the phthalocyanine dye, such as IR700 NHS ester, is protected from exposure to white light.

In some embodiments, the steps of preparing or producing a conjugate include providing antibody such as cetuximab for conjugation with the phthalocyanine dye, such as IR700. In some embodiments, the antibody is prepared prior to conjugation with the phthalocyanine dye. In some embodiments, preparing the antibody includes concentrating or diluting the antibody to a particular amount or concentration prior to the conjugation reaction. In some embodiments, preparing the antibody includes exchanging the antibody into a buffer, such as a buffer that is compatible or suitable for the conjugation reaction. In some embodiments, preparing antibody includes adjusting the pH to a pH suitable for use in the conjugation reaction. For example, the antibody is prepared at a pH that is between or between about 6 and 10, such as between or between about 8 and 9, such as about 8.5, such as 8.46.

In some embodiments, the antibody, is buffer exchanged into a buffer, such as using ultrafiltration/diafiltration such as using tangential flow filtration (TFF). In some embodiments, the TFF comprises a regenerated membrane, such as a regenerated cellulose membrane. In some embodiments, the buffer into which the antibody is exchanged is a sodium phosphate buffer, such as 100 mM sodium phosphate, such as with a pH of 8.5 or pH 8.65. In some embodiments, tangential flow filtration is performed until a desired pH of the filtrate is reached. In some embodiments, the desired pH is between or between about 6 and 10, such as between or between about 8 and 9, such as about 8.5, such as 8.46.

In some embodiments, the antibody is provided in an amount that is between or between about 0.01 g and at or about 100 g, between at or about 1 g and at or about 50 g, between at or about 1 g and at or about 25 g, between at or about 5 g and at or about 15 g, or is at or about 12 g. In some embodiments, the volume of antibody preparation is between at or about 0.01 L and at or about 100 L, between at or about 1 L and at or about 50 L, between about 1 L and at or about 15 L, or is at or about 6 L. In some embodiments, the concentration of the antibody, is less than at or about 0.01 mg/mL, or is between at or about 0.1 mg/mL and at or about 100.0 mg/mL, between at or about 0.1 mg/mL and at or about 50 mg/mL, between at or about 0.1 mg/mL and at or about 10 mg/mL, or between at or about 1 mg/mL and at or about 5 mg/mL, or is at or about 5 mg/mL or is at or about 4.5 mg/mL, or is at or about 2 mg/mL, is at or about 10 mg/mL. In some embodiments, the antibody, is diluted, such as to a concentration between at or about 0.1 mg/mL and at or about 100.0 mg/mL, between at or about 0.1 mg/mL and at or about 50 mg/mL, between at or about 0.1 mg/mL and at or about 10 mg/mL, between at or about 1 mg/mL and at or about 5 mg/mL, or between at or about 1.8 mg/mL and at or about 2.4 mg/mL, or is diluted to a concentration of or of about 2 mg/mL, about 5 mg/ml or about 10 mg/mL.

In some embodiments, the antibody is filtered through a sterile filter, such as a 0.2 μm filter or 0.22 μm filter. In some embodiments, the prepared antibody is stored, such as at a temperature below 30° C., such as generally below 26° C., 20° C., 15° C., 10° C., such as generally between at or about 2 and at or about 8° C. In some embodiments, the weight of the antibody is determined.

In some embodiments, the methods of manufacturing a conjugate include a step of contacting an antibody with a phthalocyanine dye such as IR700. In some embodiments, the phthalocyanine dye and antibody are mixed together in a container, such as a reaction vessel. In some embodiments, the contacting step is carried out in a container or vessel, such as a reaction vessel. In some embodiments, the vessel is a tube a bottle, or a carboy. In some embodiments, the vessel has a maximum volume of about or at least 1 L, 2 L, 5 L, 10 L, 15 L, 20 L, 30 L, 40 L, 50 L or 100 L. In some embodiments, the vessel is a 40 L carboy. In some embodiments, the vessel has a maximum volume of about or at least 100 μL, 500 μL, 1 mL, 1.5 mL, 5 mL, 15 mL, 50 mL, 250 mL, or 500 mL. In some embodiments, the container or vessel is translucent or opaque, is green or amber, and/or is covered, such as wrapped, in an opaque, such as aluminum, foil.

In some embodiments, the amount of dye used for contacting the antibody is calculated based on the weight of the antibody present in the container or vessel. For example, in some embodiments, an amount of dye is added such that the final molar ratio of dye to at or about antibody is from at or about 1:1 to at or about 1000:1, from at or about 1:1 to at or about 100:1, from at or about 1:1 to at or about 10:1, from at or about 1:1 to at or about 4:1, or about 4:1 or 4:1.

In some embodiments, the ratio of dye to antibody is chosen such that a desired number of dye residues are incorporated per antibody. In some embodiments, the desired number of dye residues per antibody is from or from about 1 to 5, from or from about 2-5, from or from about 2-3, or is about 3 or 3.

In some embodiments, the dye and the antibody are contacted at a controlled temperature, or are contacted in a unit with a controlled temperature, such as an incubator or refrigerator. In some embodiments, the method involves contacting the phthalocyanine dye (e.g. IR700) and the antibody at a temperature in a range from at or about 4° C. to at or about 37° C., such as from at or about 10° C. to at or about 30° C., from at or about 20° C. to at or about 30° C., or from at or about 23° C. to at or about 27° C., or that is about 25° C.+2.0° C., 25° C.+1.0° C. or 25° C.+0.3° C., such as that is at or about 25° C. In some embodiments, the contacting step is carried out at room temperature, such as between 21° C. and 25° C., such as about 23° C.

In some embodiments, the contacting step includes incubating, such as reacting, the dye and antibody. In some embodiments, the contacting can be carried out in a reaction vessel. In some embodiments, the contacting includes mixing, for example by stirring, the combined dye and antibody compositions for at least a portion of the contacting. In some embodiments, the contents are stirred, such as on a stir plate. In some embodiments, the contents are stirred for about or at least 5 to 30 minutes, such as about 5 to 20 minutes, such as about 10 to 15 minutes.

In some embodiments, the contacting step is carried out for at least 5 minutes, at least 15 minutes, at least 30 minutes, at least 60 minutes, at least 90 minutes, at least 120 minutes, at least 240 minutes, at least 360 minutes, at least 24 hours, at least 72 hours, or at least 120 hours. In some embodiments, the contacting step is carried out for 5 minutes to 150 hours, 5 minutes to 100 hours, 5 minutes to 48 hours, 5 minutes to 24 hours, 5 minutes to 6 hours, 5 minutes to 2 hours, 5 minutes to 90 minutes, 5 minutes to 60 minutes, 5 minutes to 30 minutes, 30 minutes to 150 hours, 30 minutes to 100 hours, 30 minutes to 48 hours, 30 minutes to 24 hours, 30 minutes to 6 hours, 30 minutes to 2 hours, 30 minutes to 90 minutes, 30 minutes to 60 minutes, 60 minutes to 150 hours, 60 minutes to 100 hours, 60 minutes to 48 hours, 60 minutes to 24 hours, 60 minutes to 6 hours, 60 minutes to 2 hours, 60 minutes to 90 minutes, 90 minutes to 150 hours, 90 minutes to 100 hours, 90 minutes to 48 hours, 90 minutes to 24 hours, 90 minutes to 6 hours, 90 minutes to 2 hours, 2 hours to 150 hours, 2 hours to 100 hours, 2 hours to 48 hours, 2 hours to 24 hours, 2 hours to 6 hours, 6 hours to 150 hours, 6 hours to 100 hours, 6 hours to 48 hours, 6 hours to 24 hours, 24 hours to 150 hours, 24 hours to 100 hours, 24 hours to 48 hours, 48 hours to 150 hours, 48 hours to 100 hours or 100 hours to 150 hours. In some embodiments, the contacting is carried out for a time that is from 5 minutes to 6 hours, such as 5 minutes to 4 hours, 5 minutes to 2 hours, 5 minutes to 60 minutes, 5 minutes to 30 minutes, such as about 5 minutes to 20 minutes, such as about 10 minutes to 15 minutes. In some embodiments, the method includes contacting, such as by an incubation of the phthalocyanine dye (e.g. IR700) and the antibody (e.g. antibody), for at least or about 15 minutes, at least or about 30 minutes, at least or about 60 minutes, at least or about 90 minutes, at least or about 120 minutes, or at least or about 150 minutes. In some embodiments, the method includes contacting, such as reacting the dye and the antibody for between or between about 90 and 150 minutes, such as 120 minutes.

In some embodiments, the dye and the antibody are mixed in aqueous buffer that can include an organic solvent, such as DMSO or DMF. In some embodiments, the solvent is a water-based solvent. In some embodiments, the pH of the buffer is between or between about 6 and 10, such as between or between about 7 and 10, between or between about 8 and 10, or between or between about 8 and 9.

In some embodiments, following the contacting step, the reaction is quenched, such as by adding a quenching agent, such as glycine. The term “quenching” refers to the process by which an unreacted reactive group is reacted with an excess of non-specific quenching agent (also called quencher), such as to stop the reaction between the dye and antibody. The particular agent or quencher that is used depends on the particular reactive group associated with the dye. For example, NETS-ester crosslinking reactions can be quenched in the in presence of buffers containing amines, such as buffers containing Tris or glycine.

In some embodiments, the quenching step removes any unreacted dye. In some embodiments, the quenching step removes any dye that has reacted with a non-lysine position on the antibody, such as to form a thiol ester or tyrosine ester. In some embodiments, the quenching step is performed such that substantially all of the dye reacted with the antibody is at one or more lysine positions on the antibody.

In some embodiments, the amount of quenching agent added is at least or about 200 mM, at least or about 500 mM, at least or about 1 M, at least or about 2 M, at least or about 5 M, or at least or about 10 M. In some embodiments, the quenching reaction involves the addition of 1 M glycine. In some embodiments, the final concentration of the quenching reagent after it is added to the conjugation reaction is at least or about 1 mM, at least or about 2 mM, at least or about 3 mM, at least or about 4 mM, at least or about 5 mM, or at least or about 10 mM. In some embodiments, the final concentration of the quenching regent, such as glycine, is at or about 4.2 nM. In some embodiments, the pH of the quenching step is between or between about 6 and 10, such as between or between about 7 and 10, between or between about 8 and 10, or between or between about 8 and 9. In some embodiments, the pH of the quenching step is at or about 8.0, 8.1, 8.2, 8.3, 8.4, 8.5, 8.6, 8.7, 8.8, 8.9 or 9.0.

In some embodiments, during the quenching step, the contents of the reaction vessel are mixed, such as stirred, such as on a stir plate. In some embodiments, the contents of the reaction vessel are stirred at between or between about 100 rpm and 1000 rpm, between or between about 200 rpm and 500 rpm, or are stirred at 300+50 rpm, or at 300 rpm. In some embodiments, the quenching reaction is mixed for at least or about 5 minutes, at least or about 10 minutes, or at least or about 15 minutes. In some embodiments, the quenching reaction is mixed for about 10 to 12 minutes.

In some embodiments, following the mixing of the quenching reaction, the container, such as the reaction vessel, is returned to a controlled temperature, such as in an incubator. In some embodiments, the contents of the vessel are incubated, such as from at or about 21° C. to at or about 30° C., such as from at or about 23° C. to at or about 27° C., such as at or about 25° C. In some embodiments, the incubation, such as additional incubation following the mixing of the quenching reagent with the contents of the reaction vessel, of the quenching step is carried out for at least or about 30 minutes, 60 minutes, 2 hours, 3 hours, 4 hours, 5 hours, 6 hours, 7 hours, 8, hours, 9 hours, 10 hours, 11 hours, 12 hours, 12-15 hours, 12-16 hours, 15-20 hours, 12-24 hours, about 20 hours, or about 24 hours. In some embodiments, the incubation is carried out for 12 or for about 12 hours or overnight.

In some embodiments, provided herein is a method of manufacturing a stable conjugate comprising: a) contacting a cetuximab with IR700 dye under conditions to produce a conjugate comprising IR700 dye conjugated at one or more lysines of the cetuximab selected from the group consisting of K145 (light chain), K215 (heavy chain), K292 (heavy chain), K416 (heavy chain) and K449 (heavy chain); b) subjecting the conjugate to a step during and/or subsequent to conjugation which substantially reduces IR700 dye non-specifically associated with the cetuximab; c) formulating the conjugate in a pharmaceutically acceptable buffer, wherein in each of steps a)-c) the only light to which the dye and conjugate are exposed has a wavelength within a range of about 400 nm to about 650 nm or has an intensity of less than 500 lux. In some aspects, step b) comprises performing the conjugation reaction between the IR700 dye and the cetuximab at a pH of about 8.4. In some aspects, step b) comprises subjecting the conjugate to a glycine quenching reaction at a pH of about 8.4 after completion of the conjugation reaction between the IR700 dye and the cetuximab. In some aspects, the quenching reaction is performed overnight or for a duration of greater than about 6 hours.

In some embodiments, the methods of manufacturing provided herein include a step or steps in which the conjugate is formulated, purified, or isolated to produce a drug substance. In some embodiments, the conjugate is formulated to a concentration within a range from at or about 0.1 mg/mL to at or about 1000 mg/mL, 0.1 mg/mL to about 500 mg/mL, 0.1 mg/mL to about 200 mg/mL, 0.1 mg/mL to about 100 mg/mL, 0.1 mg/mL to about 50 mg/mL, 0.1 mg/mL to about 10 mg/mL, 0.5 mg/mL to about 10 mg/mL, or 0.5 mg/mL to about 5 mg/mL.

In some embodiments, methods of formulating the conjugate can include concentrating or diluting the conjugate, exchanging the conjugate into a pharmaceutically acceptable buffer, or sterile processing.

In some embodiments, the formulating step includes concentrating the conjugate. In some embodiments, the concentrating step includes reducing the volume of the conjugate. In some embodiments, the volume reduction is achieved using an ultrafiltration/diafiltration system. In some embodiments, the volume of the conjugate is reduced from or from about 10 L, 15 L, 20 L, 25 L, 30 L, 40 L, or 50 L, to or to about 5 L, 8 L, 9 L, 10 L, 12 L or 15 L. In some embodiments, the final volume after concentration is between or between about 8 L and 10 L. In some embodiments, the conjugate is concentrated to a concentration within a range from or from about 0.1 mg/mL to about 1000 mg/mL, 0.1 mg/mL to about 500 mg/mL, 0.1 mg/mL to about 200 mg/mL, 0.1 mg/mL to about 100 mg/mL, 0.1 mg/mL to about 50 mg/mL, 0.1 mg/mL to about 10 mg/mL, 0.5 mg/mL to about 10 mg/mL, 0.5 mg/mL to about 5 mg/mL, or 1.8 mg/mL to about 2.1 mg/mL. In some embodiments, the conjugate is concentrated to or to about 2.0 mg/mL, about 5.0 mg/mL or about 10 mg/mL.

In some embodiments, the formulating step includes diluting the conjugate. In some embodiments, dilution of the conjugate involves increasing the volume of the buffer comprising the conjugate, such as from or from about 5 L, 10 L, 15, L, 20 L, 30 L, 40 L, or 50 L, to or to about 20 L, 30 L, 40 L, 50 L, or 75 L. In some embodiments, the conjugate is diluted to a concentration within a range from or from about 0.1 mg/mL to about 1000 mg/mL, 0.1 mg/mL to about 500 mg/mL, 0.1 mg/mL to about 200 mg/mL, 0.1 mg/mL to about 100 mg/mL, 0.1 mg/mL to about 50 mg/mL, 0.1 mg/mL to about 10 mg/mL, 0.5 mg/mL to about 10 mg/mL, or 0.5 mg/mL to about 5 mg/mL.

In some embodiments, the formulating step includes purifying the conjugate. In some embodiments, the conjugate is purified by gel permeation chromatography using equipment such as a SEPHADEX G-50 column, or by dialysis to remove unconjugated dye. In some embodiments, the conjugate is ultrafiltered or diafiltered, such as by using tangential flow filtration (TFF). In some embodiments, ultrafiltration/diafiltration is performed under dark or light-protected conditions to avoid exposure of the conjugate to environmental light.

In some embodiments, the formulating step includes exchanging the phthalocyanine dye-antibody conjugate (such as IR700-antibody conjugate) from the reaction buffer to a pharmaceutically acceptable buffer. In some embodiments, the buffer exchange may be carried out by ultrafiltration/diafiltration.

In some embodiments, the conjugate is formulated in a pharmaceutically acceptable buffer, such as that containing a pharmaceutically acceptable carrier or vehicle. Generally, the pharmaceutically acceptable carriers or vehicles, such as those present in the pharmaceutically acceptable buffer, are can be any known in the art. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds. The compositions containing the conjugates can be formulated into suitable pharmaceutical preparations such as solutions, suspensions, tablets, dispersible tablets, pills, capsules, powders, sustained release formulations or elixirs, for oral administrate, as well as transdermal patch preparation and dry powder inhalers. Typically, the compositions containing the compounds are formulated into pharmaceutical compositions using techniques and procedures well known in the art (see e.g., Ansel Introduction to Pharmaceutical Dosage Forms, Fourth Edition, 1985, 126). Generally, the mode of formulation is a function of the route of administration. Pharmaceutical compositions comprising phthalocyanine dye-antibody conjugates can be formulated for example, as described in PCT/US2019/035053, published as WO2019/035053.

In some embodiments, the pH of the composition is between or between about 6 and 10, such as between or between about 6 and 8, between or between about 6.9 and 7.3, such as about pH 7.1. In some embodiments, the pH of the pharmaceutically acceptable buffer is at least or about 5, at least or about 6, at least or about 7, at least or about 8, at least or about 9 or at least or about 10, or is 7.1.

In some embodiments, provided is a dye conjugate, such as a cetuximab-IR700 conjugate, that is stable. In some embodiments, by practice of the provided methods, the purity, impurities, integrity, composition and potency of the conjugate are not changed greater than acceptable specifications for manufacturing purposes to support clinical or commercial uses. In embodiments, the conjugate is stable and exhibits minimal aggregation and retains potency and activity, such as after processing, manufacture or storage of the dye. In some embodiments, the dye conjugate is stable for greater than three months, four months, five months, such as generally for greater than 6 months, greater than 7 months, 8 months, 9 months, 10 months, 11 months, 12 months or more. In some embodiments, such stability is present when stored for the time at a temperature that is less than 30° C., such as generally at a temperature that is at 2-8° C.

With reference to the dye conjugate, such as a cetuximab-IR700 conjugate, the term “stable” refers to a conjugate in which, after storage for greater than a requisite time, such as greater than three months, for example greater than or greater than about 6 months, 12 months or 24 months, greater than or greater than about 90% of the conjugate is present as a main monomer component as a percentage of the total molecular weight of the conjugate present in the sample, no more than 10.0% of the conjugate exists as a high molecular weight component as a percentage of the total molecular weight of the conjugate present in the sample or the conjugate retains at least 20% and up to 100% of its integrity, such as its physical and functional qualities, including one or more of its purity (e.g. percent monomer content vs. aggregates, such as content of higher molecular weight components), identity (e.g. chemical composition, such as structural characteristics), potency (e.g., concentration or amount required to produce a pharmacologic response) or activity (e.g., PIT killing) compared to the conjugate prior to the storage for the requisite time (e.g., t=0). In some embodiments, at least about 80%, 81%, 82%, 83%, 84%, 85%, 86%, 87%, 88%, 89%, 90%, 91%, 92%, 93%, 94%, 95%, 96%, 97%, 98% or 99% of the purity, identity, potency or activity is retained.

Also provided herein is a stable composition of conjugates manufactured by the method of any of the preceding embodiments. In some embodiments, the stable composition of conjugates comprises a population of cetuximab molecules conjugated to IR700 dye, wherein when the stable conjugate is analyzed by mass spectrometry, the ratio among peptides that contain IR700 dye conjugation at light chain lysine 145 (K145), peptides that contain IR700 dye conjugation at heavy chain lysine 215 (K215), peptides that contain IR700 dye conjugation at heavy chain lysine 292 (K292), and peptides that contain IR700 dye conjugation at heavy chain lysine 416 (K416) is about 1:1:1:1 (e.g., with reference to position numbering in the heavy chain sequence set forth in SEQ ID NO:1 and/or the light chain sequence set forth in SEQ ID NO:2).

In some embodiments, in the stable composition of conjugates, the ratio of IR700 dye conjugated to lysines in the population of cetuximab molecules is about 2:1 to about 1:2, optionally about 1:1, between positions lysine 145 (K145) in the light chain and lysine 215 (K215) in the heavy chain. In some embodiments, in the stable composition of conjugates, the ratio of IR700 dye conjugated to lysines in the population of cetuximab molecules is about 2:1 to about 1:2, optionally about 1:1, between positions lysine 145 (K145) in the light chain and lysine 292 (K292) in the heavy chain. In some embodiments, in the stable composition of conjugates, the ratio of IR700 dye conjugated to lysines in the population of cetuximab molecules is about 2:1 to about 1:2, optionally about 1:1, between positions lysine 145 (K145) in the light chain and lysine 336 (K336) in the heavy chain. In some embodiments, in the stable composition of conjugates, the ratio of IR700 dye conjugated to lysines in the population of cetuximab molecules is about 2:1 to about 1:2, optionally about 1:1, between positions lysine 145 (K145) in the light chain and lysine 416 (1(416) in the heavy chain. In some embodiments, in the stable composition of conjugates, the ratio of IR700 dye conjugated to lysines in the population of cetuximab molecules is about 2:1 to about 1:2, optionally about 1:1, between positions lysine 145 (K145) in the light chain and lysine 449 (K449) in the heavy chain.

In some embodiments, in the stable composition of conjugates, the ratio of IR700 dye conjugated to lysines in the population of cetuximab molecules is about 2:1 to about 1:2, optionally about 1:1, between positions lysine 215 (K215) in the heavy chain and lysine 292 (K292) in the heavy chain. In some embodiments, in the stable composition of conjugates, the ratio of IR700 dye conjugated to lysines in the population of cetuximab molecules is about 2:1 to about 1:2, optionally about 1:1, between positions lysine 215 (K215) in the heavy chain and lysine 336 (K336) in the heavy chain. In some embodiments, in the stable composition of conjugates, the ratio of IR700 dye conjugated to lysines in the population of cetuximab molecules is about 2:1 to about 1:2, optionally about 1:1, between positions lysine 215 (1(215) in the heavy chain and lysine 416 (1(416) in the heavy chain. In some embodiments, in the stable composition of conjugates, the ratio of IR700 dye conjugated to lysines in the population of cetuximab molecules is about 2:1 to about 1:2, optionally about 1:1, between positions lysine 215 (K215) in the heavy chain and lysine 449 (K449) in the heavy chain.

In some embodiments, in the stable composition of conjugates, the ratio of IR700 dye conjugated to lysines in the population of cetuximab molecules is about 2:1 to about 1:2, optionally about 1:1, between positions lysine 292 (K292) in the heavy chain and lysine 336 (K336) in the heavy chain. In some embodiments, in the stable composition of conjugates, the ratio of IR700 dye conjugated to lysines in the population of cetuximab molecules is about 2:1 to about 1:2, optionally about 1:1, between positions lysine 292 (K292) in the heavy chain and lysine 416 (1(416) in the heavy chain. In some embodiments, in the stable composition of conjugates, and/or the ratio of IR700 dye conjugated to lysines in the population of cetuximab molecules is about 2:1 to about 1:2, optionally about 1:1, between positions lysine 292 (K292) in the heavy chain and lysine 449 (K449) in the heavy chain.

In some embodiments, in the stable composition of conjugates, the ratio of IR700 dye conjugated to lysines in the population of cetuximab molecules is about 2:1 to about 1:2, optionally about 1:1, between positions lysine 336 (K336) in the heavy chain and lysine 416 (K416) in the heavy chain. In some embodiments, in the stable composition of conjugates, the ratio of IR700 dye conjugated to lysines in the population of cetuximab molecules is about 2:1 to about 1:2, optionally about 1:1, between positions lysine 336 (K336) in the heavy chain and lysine 449 (K449) in the heavy chain.

In some embodiments, in the stable composition of conjugates, the ratio of IR700 dye conjugated to lysines in the population of cetuximab molecules is about 2:1 to about 1:2, optionally about 1:1, between positions lysine 416 (K416) in the heavy chain and lysine 449 (K449) in the heavy chain.

In some embodiments, provided is a dye conjugate composition that is consistent. With reference to the dye conjugate composition, the term “consistent” refers to a composition that after the quenching step, has low or substantially no free dye in the composition and that substantially no free dye is released over time in the composition. For example, less than about 5%, 4%, 3%, 2%, 1%, 0.5% or 0.1% free dye is present in the composition after the quenching step and the amount of free dye in the composition does not increase during storage of the composition, including storage for about 1 month, 2 months, 3 months, 4 months, 5 months, 6 months, 7 months, 8 months, 9 months, 10 months, 11 months, 12 months or at least 1 year, 2 years or greater than 2 years.

The term “consistent” as referring to the manufacturing of a composition of conjugates refers to the substantial uniformity of the specific positions on the light chain and/or the heavy chain that are conjugated with phthalocyanine dye, such IR700, between different manufacturing batches. For example, in manufacturing the compositions described herein of cetuximab-IR700 conjugate, the predominant lysine positions conjugated with dye and the proportionality between conjugation at each lysine position is relatively constant between manufacturing batches.

### V. METHODS OF TREATMENT

Provided compositions, such as pharmaceutical compositions containing a phthalocyanine dye-antibody conjugate, such as the IR700-cetuximab conjugates described herein, can be used in therapeutic methods or treatment regimen, such as in photoimmunotherapy (PIT) methods. Photoimmunotherapy is a molecular targeted therapy that utilizes a target-specific photosensitizer based on IR700 phthalocyanine dye, conjugated to a targeting molecule, e.g., the cetuximab, targeting the epidermal growth factor receptor (EGFR; ErbB-1; HER1; EGF receptor) on the cell surface protein on tumor cells. Methods and uses of the provided conjugates and compositions, such as pharmaceutical compositions, include therapeutic methods and uses, for example, involving administration of the molecules to a subject having a disease, condition or disorder, followed by irradiation to PIT, thereby resulting in photolysis of such cells or pathogens to effect treatment of the disease or disorder. In some embodiments, the methods can be used for treating a tumor or a cancer, whereby an administered phthalocyanine-dye cetuximab conjugate (such as IR700-cetuximab conjugate) is targeted to a cell associated with a tumor, thereby resulting in photolysis of such cell and, in some cases, resulting in treatment of the tumor. Uses include uses of the compositions in such methods and treatments, such as therapeutic regimens or treatment regimens, and uses of such compositions in the preparation of a medicament in order to carry out such therapeutic methods. In some embodiments, the methods and uses thereby treat the disease or condition or disorder, such as a tumor or cancer, in the subject.

Provided herein are methods of PIT with cetuximab-IR700 conjugates. Cetuximab targets the EGFR on the surface of tumor-specific cells. The activation of the dye-conjugate by irradiation with absorbing light excites the photosensitizer and results in cell killing of the tumor cells. Generally, targeted phototoxicity appears to be primarily dependent on binding of the dye-conjugate to the cell membrane via the specific targeting molecule (e.g., antibody). For example, studies using an exemplary antibody-IR700 molecule indicate that the conjugate must be bound to the cellular membrane to be active, and that cell killing does not require intracellular localization to be effective (see. e.g., U.S. Pat. No. 8,524,239 and U.S. published application No. US20140120119). Photo-activation of the conjugate-bound cells results in rapid cell death and necrosis.

Typically, PIT results in cell death primarily of those cells to which the phthalocyanine-dye conjugate, such as antibody-IR700 conjugate, binds after the cells are irradiated, while cells that do not express the cell surface protein recognized the antibody are not killed in significant numbers. Thus, because the therapy is targeted specifically to disease cells, such as tumor cells, its effects are highly selective to disease tissue compared to healthy tissue or cells.

Provided herein are compositions, including pharmaceutical compositions for targeting the epidermal growth factor receptor (EGFR; ErbB-1; HER1; EGF receptor), such as EGFR on cancer cells. For example, the EGF receptor HER1 is typically found in adenocarcinomas, which can be found in many organs, such as the pancreas, breast, prostate, and colon.

In some embodiments, the method includes treating a pre-cancerous lesion or cancer with EGFR antibody-phthalocyanine conjugates such as cetuximab-IR700 conjugates having IR700 dye conjugated at specific lysine residues or lysine positions on the light and/or the heavy chain of the antibody for a cancer that where the cancer expresses epidermal growth factor receptor (EGFR or HER1) antigens. Such cancers may include, but are not limited to, head and neck cancer, pre-malignant dysplasia, glioblastoma, esophageal cancer, laryngeal cancer, medullary thyroid cancer, non-melanoma cutaneous SCC, breast cancer, non-small cell lung cancer (NSCLC), stomach cancer, colorectal cancer, kidney cancer, bladder cancer, pancreatic cancer, ovarian cancer, endometrial cancer, cervical cancer, vulvar cancer, prostate cancer, penile cancer, testicular cancer and anal cancer.

In some embodiments, the method includes treating a head or neck cancer with the cetuximab-IR700 conjugates or a composition containing a population of cetuximab-IR700 conjugates described herein. In some embodiments, the method of treating unwanted cells from a head or neck cancer in a subject includes: (a) administering a composition comprising a cetuximab-IR700 conjugate or population of cetuximab-IR700 conjugates with specific positions conjugated to dye as described herein to a subject, and (b) irradiating the unwanted cells at a wavelength of 660 to 740 nm at a dose of at least 1 J cm⁻² or 1 J/cm of fiber length thereby removing or killing the unwanted cell in the subject. In some embodiments, the method includes administering the cetuximab-IR700 conjugate from any of the light-protected device provided herein and/or wherein prior to and during the administration step the composition is not exposed to an intensity of environmental light greater than 500 lux. In some embodiments, the irradiation is performed at 690 nm or at 690 nm±50 nm. In some embodiments, the conjugate is administered in an amount that is at or about 160 mg/m², 320 mg/m², 640 mg/m² or 1280 mg/m².

In some embodiments the method of treating a head or neck cancer in a subject includes: (a) intravenously administering a composition comprising a cetuximab-IR700 conjugate or population of cetuximab-IR700 conjugates with specific positions conjugated to dye as described herein to a subject having a head or neck cancer as a formulated composition wherein the conjugate is administered in an amount that is at or about 640 mg/m²; and b) after administering the conjugate, irradiating the lesion at a wavelength of 690±20 nm at a dose of at least or about at least or about 50 J cm⁻² or 100 J/cm of fiber length, thereby treating the cancer in the subject. In some embodiments of the method, light illumination is administered for irradiating the lesion at or at about 20, 21, 22, 23, 24, 25, 26, 27, 28, 29, 30, 31, or 32 hours following the administration of the cetuximab-IR700 or administered at about 24 hours±4 hours following the administration of the cetuximab-IR700.

In some embodiments, provided herein is a method of killing a tumor cell comprising: administering a pharmaceutical composition comprising a conjugate disclosed herein to a site at or proximal to a tumor cell; irradiating an area proximal to the tumor cell at a wavelength of 600 nm to 850 nm at a dose of from about 25 J cm⁻² to about 400 J cm⁻² or from about 25 J/cm of fiber length to about 500 J/cm of fiber length, thereby killing the tumor cell.

In some embodiments, provided herein is a method of killing a tumor cell comprising: administering a pharmaceutical composition comprising a composition disclosed herein to a site at or proximal to a tumor cell; irradiating an area proximal to the tumor cell at a wavelength of 600 nm to 850 nm at a dose of from about 25 J cm⁻² to about 400 J cm⁻² or from about 25 J/cm of fiber length to about 500 J/cm of fiber length, thereby killing the tumor cell.

In some embodiments, provided herein is a method of treating a disease or condition comprising: administering a pharmaceutical composition comprising a conjugate disclosed herein to the subject; irradiating an area proximal to a tumor or lesion in the subject at a wavelength of 600 nm to 850 nm at a dose of from about 25 J cm⁻² to about 400 J cm⁻² or from about 25 J/cm of fiber length to about 500 J/cm of fiber length, thereby treating the disease or condition.

In some embodiments, provided herein is a method of treating a disease or condition comprising: administering a pharmaceutical composition comprising a composition disclosed herein to the subject; irradiating an area proximal to a tumor or lesion in the subject at a wavelength of 600 nm to 850 nm at a dose of from about 25 J cm⁻² to about 400 J cm⁻² or from about 25 J/cm of fiber length to about 500 J/cm of fiber length, thereby treating the disease or condition.

In some embodiments of the method, the cetuximab-IR700 conjugate to be administered is formulated in a composition comprising a non-ionic surfactant and/or a protectant. In some embodiments, cetuximab-IR700 conjugate is formulated in a composition comprising a non-ionic surfactants such as a polysorbate, a polyethylene glycol, Triton X-100, CHAPS, or Pluronic acid (F-68), and optionally a protectant such as trehalose, sorbitol, xylitol, mannitol, or sucrose. In some embodiments, cetuximab-IR700 conjugate is formulated in a composition comprising a polysorbate and trehalose. In some embodiments, cetuximab-IR700 conjugate is formulated in a composition comprising polysorbate-80 and trehalose. In some embodiments, cetuximab-IR700 conjugate is formulated in a composition comprising 10 mM sodium phosphate, 0.02% (w/v) polysorbate-80 and 9% (w/v) trehalose at pH 7.1.

In some embodiments, the subject is a human or non-human mammal. In some embodiments, the subject is a human or veterinary subject, such as a mouse. In some embodiments, the subject is a mammal, such as a human, who has cancer, or is being treated for cancer. In some embodiments the disclosed compositions are used to treat a subject who has a tumor, such as a tumor described herein. In some embodiments, the tumor has been previously treated, such as surgically or chemically removed, and the disclosed compositions are used subsequently to kill any remaining undesired tumor cells that may remain in the subject.

The provided compositions containing a conjugate, such as a cetuximab-IR700 conjugate as described herein, can be used to treat any mammalian subject, such as a human, who has a tumor, such as a cancer, or has had such previously removed or treated. Subjects in need of the disclosed therapies can include human subjects having cancer, wherein the cancer cells express a tumor-specific protein on their surface that can specifically bind to phthalocyanine dye-antibody conjugate. For example, the disclosed compositions can be used as initial treatment for cancer either alone, or in combination with radiation or other chemotherapy. The disclosed compositions can also be used in patients who have failed previous radiation or chemotherapy. Thus, in some embodiments, the subject is one who has received other therapies, but those other therapies have not provided a desired therapeutic response. The disclosed compositions can also be used in patients with localized and/or metastatic cancer.

A. Combination Treatment with Additional Therapeutic Agents

In some embodiments, the conjugates or compositions disclosed herein are administered with and the methods herein are performed with the addition of a second therapy for the treatment of the lesion, disease, or condition, e.g., an additional therapeutic agent or anti-cancer treatment. In some embodiments, the additional therapeutic agent or second therapeutic for the treatment is an immune modulator, an anti-cancer agent or other agent, that can increase the efficacy of treating the tumor, which, in some cases, can increase the therapeutic outcome or survival of the treated subject. In some embodiments, the additional therapeutic agent or second therapeutic is an immune checkpoint inhibitor. In some embodiments, the additional therapeutic agent or second therapeutic is any described below.

Prior to, during, or following administration of the composition containing the phthalocyanine dye-antibody conjugate (e.g., cetuximab-IR700 conjugate), the subject can receive one or more other therapies. In one example, the subject receives one or more treatments to remove or reduce the tumor prior to administration of the conjugate. In some embodiments, prior to, during, or following administration of the composition containing the phthalocyanine dye-antibody conjugate, the subject receives an immune modulating agent. In some embodiments, the immune modulating agent is an immune checkpoint inhibitor.

In some embodiments of the methods and compositions herein, the phthalocyanine dye-antibody conjugate, such as cetuximab-IR700, is provided in combination with another therapeutic agent, such as one or both of an immune modulating agent or anti-cancer agent. In some embodiments, the phthalocyanine dye-antibody conjugate and other therapeutic agent can be packaged as an article of manufacture as separate compositions for administration together, sequentially or intermittently. The combinations can be packaged as a kit. In some embodiments, the therapeutic agent and phthalocyanine dye-antibody conjugate are formulated together in the same composition. In some embodiments, the therapeutic agent and phthalocyanine dye-antibody conjugate are formulated as separate compositions.

In some embodiments, the other or additional agent or agents administered, or the additional agent in a combination therapy, is an unconjugated antibody. In some embodiments, the unconjugated antibody is the same or substantially the same as the antibody of the conjugate. For example, in some embodiments, prior to administration of the composition containing the conjugate, the unconjugated antibody that targets a protein or antigen, is administered to the subject. In some embodiments, the antibody is administered up to 96 hours prior to administration of the conjugate. In some embodiments, the antibody is administered at a dose within a range from or from about 10 mg/m² to about 500 mg/m². For example, the antibody is cetuximab, and cetuximab is administered to the subject up to 96 hours prior to administration of the composition containing the conjugate.

In some embodiments, the other or additional agent or agents administered, or the additional agent in a combination therapy, is an immune modulating agent or anti-cancer agent. In some embodiments, the immune modulating agent, anti-cancer agent and/or phthalocyanine dye-antibody conjugate (e.g., cetuximab-IR700 conjugate) are formulated as separate compositions. In some embodiments, the immune modulating agent is provided as a separate composition from the phthalocyanine dye-antibody conjugate, and the two compositions are administered separately. In some embodiments, the anti-cancer agent is provided as a separate composition from the phthalocyanine dye-antibody conjugate, and the two compositions are administered separately. In some embodiments, the phthalocyanine dye-antibody conjugate (e.g., cetuximab-IR700 conjugate) is formulated with one or more stabilizing agents, where the stabilization agents are non-ionic surfactants and/or protectants, and the immune modulating agent or anti-cancer agent is administered in a separate and different formulation.

In some embodiments, the immune modulating agent and/or anti-cancer agent and the phthalocyanine dye-antibody conjugate is formulated in the same composition. The compositions can be formulated for parenteral delivery (i.e. for systemic delivery). For example, the compositions or combination of compositions are formulated for subcutaneous delivery or for intravenous delivery. The agents, such as a phthalocyanine dye-antibody conjugate, an immune modulating agent, and/or an anti-cancer agent can be administered by different routes of administration.

Examples of additional therapies that can be used in combination with the disclosed antibody-IR700 conjugates for treating cancers or tumors, which may enhance accessibility of the tumor to additional therapeutic agents, include but are not limited to, surgical treatment for removal or reduction of the tumor, such as surgical resection, cryotherapy, or chemoembolization, as well as anti-tumor pharmaceutical treatments which can include radiotherapeutic agents, anti-neoplastic chemotherapeutic agents, antibiotics, alkylating agents and antioxidants, kinase inhibitors, and other agents. In some examples, the additional therapeutic agent is conjugated to a nanoparticle. Particular examples of additional therapeutic agents that can be used include microtubule binding agents, DNA intercalators or cross-linkers, DNA synthesis inhibitors, DNA and/or RNA transcription inhibitors, antibodies, enzymes, enzyme inhibitors, and gene regulators. These agents, which are administered at a therapeutically effective amount, and treatments can be used alone or in combination. Methods and therapeutic dosages of such agents are known to those skilled in the art, and can be determined by a skilled clinician.

In some embodiments, at least a portion of the tumor, such as a metastatic tumor, is surgically removed, for example via cryotherapy, irradiated, chemically treated, for example via chemoembolization, or combinations thereof, prior to administration of the disclosed therapies, such as administration of phthalocyanine dye-antibody conjugate. For example, a subject having a metastatic tumor can have all or part of the tumor surgically excised prior to administration of the disclosed therapies. In some embodiments, one or more chemotherapeutic agents are administered following treatment with conjugate and irradiation. In some embodiments, the subject has a metastatic tumor and is administered radiation therapy, chemoembolization therapy, or both concurrently with the administration of the disclosed therapies.

In some embodiments, the immune modulating agent is or comprises an antibody or an antigen-binding fragment thereof, a small molecule or a polypeptide. In some embodiments, the immune modulating agent is or comprises the immune modulating agent specifically binds a molecule selected from among CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28, VISTA, ICOS, ICOS-L, CD27, CD30, STING, CCR4, and A2A adenosine receptor. In some embodiments, the immune modulating agent is selected from among cemiplimab, nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MED14736, MOXR0916, AMP-224, and MSB001078C, or is an antigen-binding fragment thereof. In some embodiments, the immune modulating agent is administered prior to treatment with conjugate and irradiation, such as at or at about 1 week, 2 weeks, 3 weeks or 4 weeks prior to conjugate administration. In some embodiments, the immune modulating agent is administered subsequent to treatment with conjugate and irradiation, such as at or at about 1 week, 2 weeks, 3 weeks or 4 weeks after conjugate administration. In some embodiments, the immune modulating agent is administered prior to and subsequent to treatment with conjugate and irradiation, such as on a cycle of every 1 week, 2 weeks, 3 weeks or 4 weeks.

In some embodiments, the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor, an anti-neoplastic agent, or a combination thereof. In some embodiments, the anti-cancer agent is an antibody or an antigen-binding fragment thereof, a small molecule or a polypeptide.

In some embodiments, the anti-cancer agent is selected from among one or more of 5-Fluorouracil/leukovorin, oxaliplatin, irinotecan, regorafenib, ziv-afibercept, capecitabine, cisplatin, paclitaxel, toptecan, carboplatin, gemcitabine, docetaxel, 5-FU, ifosfamide, mitomycin, pemetrexed, vinorelbine, carmustine wager, temozolomide, methotrexate, capacitabine, lapatinib, etoposide, dabrafenib, vemurafenib, liposomal cytarabine, cytarabine, interferon alpha, erlotinib, vincristine, cyclophosphamide, lomusine, procarbazine, sunitinib, somastostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanredtide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxoliplatin and auroropyrimidine. In some embodiments, the anti-cancer agent is selected from among one or more ofbevacizumab, cetuximab, panitumumab, ramucirumab, ipilimumab, rituximab, trastuzumab, ado-trastuzumab emtansine, pertuzumab, nivolumab, or an antigen-binding fragment thereof. In some embodiments, the anti-cancer agent is selected from among one or more of lapatinib, dabrafenib, vemurafenib, erlotinib, sunitinib, pazopanib, imatinib, regorafenib, sorafenib, nilotinib, dasantinib, celecoxib, crizotinib, certinib, afatinib, axitinib, bevacizumab, bosutinib, cabozantinib, afatinib, gefitinib, temsirolimus, everolimus, sirolimus, ibrutinib, imatinib, lenvatinib, olaparib, palbociclib, ruxolitinib, trametinib, vandetanib or vismodegib.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about a value” is or refers to ±25%, ±10%, ±5%, ±1%, ±0.1%, ±0.01% of the value.

As used herein, a “conjugate” refers to a polypeptide linked directly or indirectly to one or more other molecules, such as polypeptides or chemical moieties. Such conjugates include fusion proteins, those produced by chemical conjugates, and those produced by any other methods. For example, a conjugate can refer to a phthalocyanine dye, such as an IR700 molecule, linked directly or indirectly to one or more other molecules, such as an antibody.

As used herein, a composition refers to any mixture of two or more products, substances, or compounds, including cells. It may be a solution, a suspension, liquid, powder, a paste, aqueous, non-aqueous or any combination thereof.

As used herein, a “pharmaceutical composition” or “pharmaceutical formulation” refers to a preparation which is in such form as to permit the biological activity of an active ingredient contained therein to be effective, and which contains no additional components which are unacceptably toxic to a subject to which the formulation would be administered.

As used herein, a “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “unmodified peptides” refer to peptides that do not have an IR700 molecule conjugated to a lysine within the peptide. Such peptides do not exclude peptides that are otherwise modified, such as by oxidation, phosphorylation, acetylation, alkylation, glycosylation, loss of water, or other modification(s) that do not include an IR700 molecule.

As used herein, a kit is a packaged combination that optionally includes other elements, such as additional reagents and instructions for use of the combination or elements thereof.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “article of manufacture” is a product that is made and, in some cases, that can be sold. In some embodiments, the term can refer to compositions contained in articles of packaging, such as in a container.

As used herein, “disease or disorder” refers to a pathological condition in an organism resulting from cause or condition including, but not limited to, infections, acquired conditions, genetic conditions, and characterized by identifiable symptoms. Diseases and disorders of interest herein are those that are treatable by immune globulin.

As used herein, “treating” a subject with a disease or condition means that the subject's symptoms are partially or totally alleviated, or remain static following treatment. Hence treating encompasses prophylaxis, therapy, and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting, or partially arresting a symptom of a disease or disorder.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein the term “substantially” refers to a high level of similarity. In some embodiments, substantially may refer to almost all or complete, such as at least 85%, 90%, 95%, 99%, 99.9%, or 99.99% complete. For example, in some embodiments, if an agent is said to be substantially in a state, then at least 85%, 90%, 95%, 99%, 99.9%, or 99.99% of the agent is in the state.

All publications, including patent documents, scientific articles, and databases referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### VII. EXEMPLARY EMBODIMENTS

Among the embodiments provided herein are:

1. A conjugate comprising a cetuximab conjugated to an IR700 dye, wherein at least one molecule of the IR700 dye is linked to a lysine (K) in a light chain of the cetuximab.

2. The conjugate of embodiment 1, wherein the at least one molecule of the IR700 dye is linked to a lysine at a position selected from the group consisting of K107, K145, K188, K190, and K207 in the light chain.

3. The conjugate of embodiment 1 or embodiment 2, wherein the at least one molecule of the IR700 dye is linked to K145 in the light chain.

4. The conjugate of any one of embodiments 1-3, wherein the cetuximab is linked to the IR700 dye at two or more lysine positions in one or both light chains of the cetuximab.

5. The conjugate of any one of embodiments 1-4, wherein the cetuximab is linked to the IR700 dye at K145 in one or both light chains of the cetuximab.

6. The conjugate of any one of embodiments 1-5, wherein the cetuximab is linked to the IR700 dye at K145 in a light chain of the cetuximab and at least one other lysine position in a light chain or a heavy chain of the cetuximab.

7. A conjugate comprising a cetuximab conjugated to an IR700 dye, wherein at least one molecule of the IR700 dye is linked to a lysine (K) in a heavy chain of the cetuximab.

8. The conjugate of embodiment 7, wherein the at least one molecule of the IR700 dye is linked to a lysine at a position selected from the group consisting of K5, K75, K215, K248, K292, K328, K336, K416, and K449 in the heavy chain.

9. The conjugate of embodiment 7 or embodiment 8, wherein the at least one molecule of the IR700 dye is linked to a lysine at a position selected from the group consisting of K215, K292, K336, K416, and K449 in the heavy chain.

10. The conjugate of any one of embodiments 7-9, wherein the cetuximab is linked to the IR700 dye at two or more lysine positions in one or both heavy chains of the cetuximab.

11. The conjugate of any one of embodiments 7-10, wherein the cetuximab is linked to the IR700 dye at three or more lysine positions in one or both heavy chains of the cetuximab.

12. The conjugate of any one of embodiments 7-11, wherein the cetuximab is linked to the IR700 dye at K215, K292, K336, K416, and/or K449 in one or both heavy chains of the cetuximab.

13. The conjugate of any one of embodiments 7-12, wherein the cetuximab is linked to the IR700 dye at K215, K292, K336, K416, and/or K449 in a heavy chain of the cetuximab and at least one other lysine position in a light chain or a heavy chain of the cetuximab.

14. The conjugate of any one of embodiments 7-13, wherein the cetuximab is linked to the IR700 dye at K145 in one or both light chains of the cetuximab.

15. A conjugate comprising a cetuximab conjugated to an IR700 dye, wherein at least one molecule of the IR700 dye is linked to a lysine (K) in a light chain of the cetuximab, and at least one molecule of the IR700 dye is linked to a lysine (K) in a heavy chain of the cetuximab.

16. The conjugate of embodiment 15, wherein the lysine in the light chain is selected from the group consisting of K107, K145, K188, K190, and K207.

17. The conjugate of embodiment 15 or embodiment 16, wherein the lysine in the heavy chain is selected from the group consisting of K5, K75, K215, K248, K292, K328, K336, K416, and K449.

18. The conjugate of any one of embodiments 15-17, wherein the cetuximab is linked to the IR700 dye at K145 in one or both light chains of the cetuximab.

19. The conjugate of any one of embodiments 15-18, wherein the cetuximab is linked to the IR700 dye at K215, K292, K336, K416, and/or K449 in one or both heavy chains of the cetuximab.

20. The conjugate of any one of embodiments 15-19, wherein the cetuximab is linked to the IR700 dye at K145 in one or both light chains of the cetuximab, and at K215, K292, K416, and K449 in one or both heavy chains of the cetuximab.

21. The conjugate of any one of embodiments 1-20, wherein the ratio of the IR700 dye molecule to the cetuximab is 1:1, 2:1, 3:1, or 4:1.

22. The conjugate of any one of embodiments 1-21, wherein the conjugate is capable of being activated by irradiation with a wavelength between about 600 nm and about 850 nm (e.g., 690 nm±50 nm) and thereby acquires a cell killing activity.

23. A composition comprising the conjugate of any one of embodiments 1-22 and a pharmaceutically acceptable excipient.

24. A composition comprising a population of cetuximab molecules, wherein at least 50%, 60%, 70%, 80%, 90%, or more than 90% of the cetuximab molecules have at least one molecule of an IR700 dye linked to a lysine (K) in a light chain of each cetuximab molecule.

25. The composition of embodiment 24, wherein at least 50%, 60%, 70%, 80%, 90%, or more than 90% of the cetuximab molecules have at least one molecule of the IR700 dye linked to K107, K145, K188, K190, and/or K207 in one or both light chains of each cetuximab molecule.

26. The composition of embodiment 24 or embodiment 25, wherein in the population, the predominant light chain lysine position conjugated to the IR700 dye is K145.

27. The composition of any one of embodiments 24-26, wherein when the cetuximab molecules are analyzed by denatured mass spectrometry, peptides containing IR700 dye conjugation at light chain K145 are more abundant than peptides containing IR700 dye conjugated to other light chain lysine positions.

28. A composition comprising a population of cetuximab molecules, wherein at least 50%, 60%, 70%, 80%, 90%, or more than 90% of the cetuximab molecules have at least one molecule of an IR700 dye linked to a lysine (K) in a heavy chain of each cetuximab molecule.

29. The composition of embodiment 28, wherein at least 50%, 60%, 70%, 80%, 90%, or more than 90% of the cetuximab molecules have at least one molecule of the IR700 dye linked to K5, K75, K215, K248, K292, K328, K336, K416, and/or K449 in one or both heavy chains of each cetuximab molecule.

30. The composition of embodiment 28 or embodiment 29, wherein in the population, the predominant heavy chain lysine position conjugated to the IR700 dye is one or more of K215, K292, K336, K416, and K449.

31. The composition of any one of embodiments 28-30, wherein when the cetuximab molecules are analyzed by mass spectrometry, peptides containing IR700 dye conjugation at heavy chain K215, K292, K336, K416, or K449 are more abundant than peptides containing IR700 dye conjugated to other heavy chain lysine positions.

32. The composition of any one of embodiments 28-31, wherein at least 70%, 80%, 90%, or more than 90% of the cetuximab molecules have at least one molecule of the IR700 dye linked to two or more lysines in one or both heavy chains of each cetuximab molecule.

33. The composition of any one of embodiments 24-32, wherein no more than about 20% of the cetuximab molecules are unconjugated with the IR700 dye.

34. The composition of embodiment 33, wherein less than 10% of the cetuximab molecules are unconjugated with the IR700 dye, and/or the percentage of free dye in the composition is less than 3%.

35. The composition of any one of embodiments 24-34, wherein the ratio of the IR700 dye molecules in the composition to the cetuximab molecules in the population is about 2:1, about 2.5:1, or about 3:1.

36. A composition comprising a population of cetuximab molecules, wherein at least 50%, 60%, 70%, 80%, 90%, or more than 90% of the cetuximab molecules have at least one molecule of an IR700 dye linked to a lysine (K) in a light chain or a heavy chain of each cetuximab molecule.

37. The composition of embodiment 36, wherein at least 50%, 60%, 70%, 80%, 90%, or more than 90% of the cetuximab molecules have at least one molecule of the IR700 dye linked to K107, K145, K188, K190, and/or K207 in one or both light chains or linked to K5, K75, K215, K248, K292, K328, K336, K416, and/or K449 in one or both heavy chains of each cetuximab molecule.

38. The composition of embodiment 37, wherein at least 50%, 60%, 70%, 80%, 90%, or more than 90% of the cetuximab molecules have at least one molecule of the IR700 dye linked to K145 in one or both light chains or linked to K215, K292, K336, K416, or K449 in one or both heavy chains of each cetuximab molecule.

39. A composition comprising a population of cetuximab molecules, wherein at least 50%, 60%, 70%, 80%, 90%, or more than 90% of the cetuximab molecules have at least one molecule of an IR700 dye linked to a lysine (K) in a light chain and at least one molecule of the IR700 dye linked to a lysine in a heavy chain of each cetuximab molecule.

40. The composition of embodiment 39, wherein at least 50%, 60%, 70%, 80%, 90%, or more than 90% of the cetuximab molecules have at least one molecule of the phthalocyanine IR700 dye linked to K107, K145, K188, K190, and/or K207 in one or both light chains and at least one molecule of the IR700 dye linked to K5, K75, K215, K248, K292, K328, K336, K416, and/or K449 in one or both heavy chains of each cetuximab molecule.

41. The composition of embodiment 40, wherein at least 50%, 60%, 70%, 80%, 90%, or more than 90% of the cetuximab molecules have at least one molecule of the phthalocyanine IR700 dye linked to K145 in one or both light chains and at least one molecule of the IR700 dye linked to K215, K292, K336, K416, or K449 in one or both heavy chains of each cetuximab molecule.

42. A composition comprising a population of cetuximab molecules, wherein no more than about 20% of the cetuximab molecules are unconjugated with an IR700 dye at a lysine (K) in a light chain or a heavy chain of the cetuximab molecules.

43. The composition of embodiment 42, wherein less than 15% of the cetuximab molecules are unconjugated with the IR700 dye.

44. The composition of embodiment 43, wherein less than 10% of the cetuximab molecules are unconjugated with the IR700 dye.

45. The composition of any one of embodiments 42-44, wherein the percentage of free dye in the composition among all dye molecules in the composition is less than about 3%, less than about 2%, less than about 1%, or less than about 0.5%.

46. The composition of any one of embodiments 42-45, wherein the ratio of the IR700 dye molecules in the composition to the cetuximab molecules in the population is about 2:1, about 2.5:1, or about 3:1.

47. A composition comprising a population of cetuximab molecules, wherein one or more cetuximab molecules are conjugated with an IR700 dye at a lysine (K) in a light chain or a heavy chain of the one or more cetuximab molecules, the ratio of the IR700 dye molecules in the composition to the cetuximab molecules in the population is about 2:1, about 2.5:1, or about 3:1, less than 10% of the cetuximab molecules are unconjugated with the IR700 dye, and the percentage of free dye in the composition among all dye molecules in the composition is less than about 0.5%.

48. A composition comprising SEQ ID NO: 1, wherein lysine 145 of SEQ ID NO: 1 is conjugated to an IR700 dye.

49. A composition comprising SEQ ID NO: 2, wherein lysine 215, lysine 292, lysine 416, and/or lysine 449 of SEQ ID NO: 2 is or are conjugated to an IR700 dye.

50. A composition, comprising SEQ ID NO: 1 and SEQ ID No: 2, wherein lysine 145 of SEQ ID NO: 1 is conjugated to an IR700 dye and lysine 215, lysine 292, lysine 416, and/or lysine 449 of SEQ ID NO: 2 is or are conjugated to an IR700 dye.

51. The composition of embodiment 50, comprising SEQ ID NO: 1 and SEQ ID NO: 2 in the same molecule(s), wherein lysine 145 of SEQ ID NO: 1 is conjugated to an IR700 dye and lysine 215, lysine 292, lysine 416, and/or lysine 449 of SEQ ID NO: 2 is or are conjugated to an IR700 dye.

52. The composition of embodiment 50 or embodiment 51, comprising SEQ ID NO: 1 and SEQ ID NO: 2 in different molecules, wherein lysine 145 of SEQ ID NO: 1 is conjugated to an IR700 dye and lysine 215, lysine 292, lysine 416, and/or lysine 449 of SEQ ID NO: 2 is or are conjugated to an IR700 dye.

53. A composition comprising a population of cetuximab molecules, wherein less than 15% of the cetuximab molecules are unconjugated with an IR700 dye, the composition comprises less than 3% free IR700 dye, and the percentage of free dye in the composition is substantially unchanged after storage for about 6 months.

54. A composition comprising a population of cetuximab molecules conjugated with an IR700 dye, wherein the composition comprises less than or less than about 0.6%, less than or less than about 0.5%, less than or less than about 0.4%, or less than or less than about 0.3% free dye.

55. The composition of embodiment 54, wherein the composition comprises at least or at least about 95%, 96% 97% or 98% monomer.

56. The composition of embodiment 55, wherein the composition comprises less than or less than about 5%, 4% or 3% high molecular weight species.

57. The composition of any of embodiments 54-56, wherein the composition comprises less than or less than about 30%, 20%, 25%, 20%, 15% or 10% unconjugated antibody.

58. The composition of any of embodiments 54-57, wherein the percentage of free dye is substantially unchanged after storage of the composition for 6 months in dark or reduced light conditions.

59. The composition of any one of embodiments 53-58, wherein at least 70%, 80%, 90%, or more than 90% of the cetuximab molecules have at least one IR700 dye molecule conjugated to a lysine (K) in a light chain of each cetuximab molecule.

60. The composition of embodiment 59, wherein in the population, the predominant light chain lysine position conjugated to the IR700 dye is K145.

61. The composition of any one of embodiments 53-60, wherein at least 70%, 80%, 90%, or more than 90% of the population has at least one IR700 dye molecule conjugated to a lysine (K) in a heavy chain of each cetuximab molecule.

62. The composition of embodiment 61, wherein in the population, the predominant heavy chain lysine position conjugated to the IR700 dye is one or more of K215, K292, K336, K416, and K449.

63. A composition comprising a population of cetuximab molecules, wherein between about 9% and about 10% of total peptides of the cetuximab molecules analyzed by mass spectrometry are peptides that contain IR700 dye conjugation at light chain lysine 145 (K145).

64. A composition comprising a population of cetuximab molecules, wherein between about 9% and about 11% of total peptides of the cetuximab molecules analyzed by mass spectrometry are peptides that contain IR700 dye conjugation at heavy chain lysine 215 (K215).

65. A composition comprising a population of cetuximab molecules, wherein between about 9% and about 11% of total peptides of the cetuximab molecules analyzed by mass spectrometry are peptides that contain IR700 dye conjugation at heavy chain lysine 292 (K292).

66. A composition comprising a population of cetuximab molecules, wherein between about 10% and about 12% of total peptides of the cetuximab molecules analyzed by mass spectrometry are peptides that contain IR700 dye conjugation at heavy chain lysine 416 (K416).

67. A composition comprising a population of cetuximab molecules, wherein between about 7% and about 9% of total peptides of the cetuximab molecules analyzed by mass spectrometry are peptides that contain IR700 dye conjugation at heavy chain lysine 449 (K449).

68. A composition comprising a population of cetuximab molecules, wherein: between about 9% and about 10% of total peptides of the cetuximab molecules analyzed by mass spectrometry are peptides that contain IR700 dye conjugation at light chain lysine 145 (K145); between about 9% and about 11% of total peptides of the cetuximab molecules analyzed by mass spectrometry are peptides that contain IR700 dye conjugation at heavy chain lysine 215 (K215); between about 9% and about 11% of total peptides of the cetuximab molecules analyzed by mass spectrometry are peptides that contain IR700 dye conjugation at heavy chain lysine 292 (K292); between about 10% and about 12% of total peptides of the cetuximab molecules analyzed by mass spectrometry are peptides that contain IR700 dye conjugation at heavy chain lysine 416 (K416); between about 7% and about 9% of total peptides of the cetuximab molecules analyzed by mass spectrometry are peptides that contain IR700 dye conjugation at heavy chain lysine 449 (K449); and/or between about 5% and about 7% of total peptides of the cetuximab molecules analyzed by mass spectrometry are peptides that contain IR700 dye conjugation at heavy chain lysine 336 (K336).

69. The composition of any one of embodiments 63-68, wherein: between about 3% and 4% of total peptides of the cetuximab molecules analyzed by mass spectrometry are peptides that contain IR700 dye conjugation at light chain lysine 107 (K107); between about 1% and 3% of total peptides of the cetuximab molecules analyzed by mass spectrometry are peptides that contain IR700 dye conjugation at light chain lysine 188 (K188); between about 3% and 4% of total peptides of the cetuximab molecules analyzed by mass spectrometry are peptides that contain IR700 dye conjugation at light chain lysine 190 (K190); between about 1% and 3% of total peptides of the cetuximab molecules analyzed by mass spectrometry are peptides that contain IR700 dye conjugation at light chain lysine 207 (K207); between about 3% and 4% of total peptides of the cetuximab molecules analyzed by mass spectrometry are peptides that contain IR700 dye conjugation at heavy chain lysine 5 (K5); between about 3% and 4% of total peptides of the cetuximab molecules analyzed by mass spectrometry are peptides that contain IR700 dye conjugation at heavy chain lysine 75 (K75); between about 1% and 2% of total peptides of the cetuximab molecules analyzed by mass spectrometry are peptides that contain IR700 dye conjugation at heavy chain lysine 248 (K248); and/or between about 1% and 2% of total peptides of the cetuximab molecules analyzed by mass spectrometry are peptides that contain IR700 dye conjugation at heavy chain lysine 328 (K328).

70. A composition comprising a population of cetuximab molecules conjugated to IR700 dye, wherein: the ratio of IR700 dye conjugated to lysines in the population of cetuximab molecules is about 2:1 to about 1:2, optionally about 1:1, between positions lysine 145 (K145) in the light chain and lysine 215 (K215) in the heavy chain; the ratio of IR700 dye conjugated to lysines in the population of cetuximab molecules is about 2:1 to about 1:2, optionally about 1:1, between positions lysine 145 (K145) in the light chain and lysine 292 (K292) in the heavy chain; the ratio of IR700 dye conjugated to lysines in the population of cetuximab molecules is about 2:1 to about 1:2, optionally about 1:1, between positions lysine 145 (K145) in the light chain and lysine 336 (K336) in the heavy chain; the ratio of IR700 dye conjugated to lysines in the population of cetuximab molecules is about 2:1 to about 1:2, optionally about 1:1, between positions lysine 145 (K145) in the light chain and lysine 416 (K416) in the heavy chain; and/or the ratio of IR700 dye conjugated to lysines in the population of cetuximab molecules is about 2:1 to about 1:2, optionally about 1:1, between positions lysine 145 (K145) in the light chain and lysine 449 (K449) in the heavy chain.

71. A composition comprising a population of cetuximab molecules conjugated to IR700 dye, wherein: the ratio of IR700 dye conjugated to lysines in the population of cetuximab molecules is about 2:1 to about 1:2, optionally about 1:1, between positions lysine 215 (1(215) in the heavy chain and lysine 292 (K292) in the heavy chain; the ratio of IR700 dye conjugated to lysines in the population of cetuximab molecules is about 2:1 to about 1:2, optionally about 1:1, between positions lysine 215 (K215) in the heavy chain and lysine 336 (K336) in the heavy chain; the ratio of IR700 dye conjugated to lysines in the population of cetuximab molecules is about 2:1 to about 1:2, optionally about 1:1, between positions lysine 215 (K215) in the heavy chain and lysine 416 (K416) in the heavy chain; and/or the ratio of IR700 dye conjugated to lysines in the population of cetuximab molecules is about 2:1 to about 1:2, optionally about 1:1, between positions lysine 215 (K215) in the heavy chain and lysine 449 (K449) in the heavy chain.

72. A composition comprising a population of cetuximab molecules conjugated to IR700 dye, wherein: the ratio of IR700 dye conjugated to lysines in the population of cetuximab molecules is about 2:1 to about 1:2, optionally about 1:1, between positions lysine 292 (K292) in the heavy chain and lysine 336 (K336) in the heavy chain; the ratio of IR700 dye conjugated to lysines in the population of cetuximab molecules is about 2:1 to about 1:2, optionally about 1:1, between positions lysine 292 (K292) in the heavy chain and lysine 416 (K416) in the heavy chain; and/or the ratio of IR700 dye conjugated to lysines in the population of cetuximab molecules is about 2:1 to about 1:2, optionally about 1:1, between positions lysine 292 (K292) in the heavy chain and lysine 449 (K449) in the heavy chain.

73. A composition comprising a population of cetuximab molecules conjugated to IR700 dye, wherein: the ratio of IR700 dye conjugated to lysines in the population of cetuximab molecules is about 2:1 to about 1:2, optionally about 1:1, between positions lysine 336 (K336) in the heavy chain and lysine 416 (K416) in the heavy chain; and/or the ratio of IR700 dye conjugated to lysines in the population of cetuximab molecules is about 2:1 to about 1:2, optionally about 1:1, between positions lysine 336 (K336) in the heavy chain and lysine 449 (K449) in the heavy chain.

74. A composition comprising a population of cetuximab molecules conjugated to IR700 dye, wherein the ratio of IR700 dye conjugated to lysines in the population of cetuximab molecules is about 2:1 to about 1:2, optionally about 1:1, between positions lysine 416 (1(416) in the heavy chain and lysine 449 (K449) in the heavy chain.

75. A composition comprising a population of cetuximab molecules conjugated to IR700 dye, wherein the ratio of IR700 dye conjugated to lysines in the population of cetuximab molecules is about 1:1:1:1 among positions lysine 145 (K145) in the light chain, lysine 215 (K215) in the heavy chain, lysine 292 (K292) in the heavy chain, and lysine 416 (1(416) in the heavy chain.

76. A composition comprising a population of cetuximab molecules conjugated to IR700 dye, wherein the ratio of IR700 dye conjugated to lysines in the population of cetuximab molecules is about 1:1:1:1:1:1 among positions lysine 145 (K145) in the light chain, lysine 215 (K215) in the heavy chain, lysine 292 (K292) in the heavy chain, lysine 336 (K336) in the heavy chain, lysine 416 (K416) in the heavy chain, and lysine 449 (K449) in the heavy chain.

77. The composition of any one of embodiments 71-76, wherein the ratio is measured by mass spectrometry.

78. A method of killing a tumor or cancer cell, comprising: administering a pharmaceutical composition comprising the conjugate or composition of any one of embodiments 1-77 to a site at or proximal to the tumor or cancer cell; and irradiating an area proximal to the tumor cell at a wavelength of about 600 nm to about 850 nm at a dose of from about 25 J cm⁻² to about 400 J cm⁻² or from about 25 J/cm of fiber length to about 500 J/cm of fiber length, thereby killing the tumor or cancer cell.

79. A method of treating a disease or condition, e.g., a tumor or cancer, in a subject, comprising: administering a pharmaceutical composition comprising the conjugate or composition of any one of embodiments 1-78 to the subject; and irradiating an area proximal to a lesion (e.g., due to the tumor or cancer) in the subject at a wavelength of about 600 nm to about 850 nm at a dose of from about 25 J cm⁻² to about 400 J cm⁻² or from about 25 J/cm of fiber length to about 500 J/cm of fiber length, thereby treating the disease or condition.

80. The method of embodiment 78 or embodiment 79, further comprising providing the pharmaceutical composition prior to the administering step.

81. The method of any one of embodiments 78-80, wherein the irradiating step is carried out at a wavelength of 690±50 nm or at a wavelength of or about 690±20 nm.

82. The method of any one of embodiments 78-81, wherein the irradiating step is carried out at a wavelength of about 690 nm.

83. The method of any one of embodiments 78-82, wherein the tumor is a carcinoma of the bladder, pancreas, colon, ovary, lung, breast, stomach, prostate, cervix, esophagus or head and neck.

84. The method of any one of embodiments 78-83, wherein the cancer is located at the head and neck, breast, liver, colon, ovary, prostate, pancreas, brain, cervix, bone, skin, eye, bladder, stomach, esophagus, peritoneum, or lung.

85. The method of any one of embodiments 78-84, wherein the cancer is a cancer located at the head and neck.

86. A method of manufacturing a stable conjugate comprising: a) contacting a cetuximab with an IR700 dye under conditions to produce a conjugate comprising the IR700 dye linked to one or more lysines of the cetuximab selected from the group consisting of K145 (light chain), K215 (heavy chain), K292 (heavy chain), K336 (heavy chain), K416 (heavy chain), and K449 (heavy chain); b) subjecting the conjugate to a step during and/or subsequent to conjugation which substantially reduces the IR700 dye non-specifically associated with the cetuximab; c) formulating the conjugate in a pharmaceutically acceptable buffer, wherein in each of steps a)-c) the only light to which the dye and conjugate are exposed has a wavelength within a range of about 400 nm to about 650 nm or has an intensity of less than 500 lux.

87. The method of embodiment 86, wherein step b) comprises subjecting the conjugate to a quenching reaction.

88. The method of embodiment 86 or embodiment 87, wherein step b) comprises subjecting the conjugate to a glycine quenching reaction after completion of the conjugation reaction between the IR700 dye and the cetuximab.

89. The method of embodiment 88, wherein the quenching reaction is performed overnight or for a duration of greater than about 6 hours.

90. A stable conjugate manufactured by any one of embodiments 86-89.

91. The stable conjugate of embodiment 90, which comprises a population of cetuximab molecules conjugated to IR700 dye, wherein the ratio of IR700 dye conjugated to lysines of cetuximab molecules in the population is about 1:1:1:1 among positions lysine 145 (K145) in the light chain, lysine 215 (K215) in the heavy chain, lysine 292 (K292) in the heavy chain, and lysine 416 (K416) in the heavy chain.

92. A conjugate comprising at least two molecules of IR700 conjugated to at least two lysine (K) positions in a cetuximab, and wherein the at least two lysine positions are independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (1(75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab.

93. The conjugate of embodiment 92, comprising at least three molecules of IR700 conjugated to at least three lysine positions in the cetuximab.

94. The conjugate of embodiment 93, wherein the at least three lysine positions are independently selected from the group consisting of K107, K145, K188, K190, and K207 in the light chain and K5, K75, K215, K248, K292, K328, K336, K416, and K449 in the heavy chain.

95. The conjugate of any of embodiments 92-94, wherein at least one of the lysine positions conjugated to IR700 is selected from the group consisting of K145 in the light chain or K215, K416 or K449 in the heavy chain.

96. The conjugate of any of embodiments 92-95, wherein at least one molecule of IR700 is conjugated to a lysine in the light chain, and at least one molecule of IR700 is conjugated to a lysine in the heavy chain.

97. The conjugate of any of embodiments 92-96, wherein the conjugate is capable of being activated by light irradiation with a wavelength between 690 nm±50 nm and thereby exhibits a cell killing activity when the conjugate is bound to an epitope on the surface of the cell.

98. A composition comprising the conjugate of any of embodiments 92-97 and a pharmaceutically acceptable excipient.

99. A composition comprising a population of conjugates, wherein the conjugates in the population comprise IR700 conjugated to a cetuximab, wherein at least at or about 50%, 60%, 70%, 80%, 90%, or more than at or about 90% of the conjugates comprise at least two molecules of IR700 conjugated to at least two lysine (K) positions in the cetuximab, and wherein the two lysine positions are independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab.

100. The composition of embodiment 98 or 99, wherein at least at or about 50%, 60%, 70%, 80%, 90%, or more than at or about 90% of the conjugates comprise at least three molecules of IR700 conjugated to at least three lysine positions in the cetuximab.

101. The composition of embodiment 100, wherein the at least three lysine positions are independently selected from the group consisting of K107, K145, K188, K190, and K207 in the light chain and K5, K75, K215, K248, K292, K328, K336, K416, and K449 in the heavy chain.

102. The composition of any of embodiments 98-101, wherein at least at or about 50%, 60%, 70%, 80%, 90%, or more than at or about 90% of the conjugates comprise at least one molecule of IR700 conjugated to K145 in the light chain or K215, K416 or K449 in the heavy chain.

103. The composition of any of embodiments 98-102, wherein at least at or about 50%, 60%, 70%, 80%, 90%, or more than at or about 90% of the conjugates comprise a molecule of IR700 conjugated to K145 in the light chain and a molecule of IR700 conjugated to at least one of K215, K416 or K449 in the heavy chain.

104. The composition of any of embodiments 98-103, wherein the ratio of IR700 molecule to the cetuximab is between about 2:1 to about 4:1.

105. The composition of any of embodiments 98-104, wherein the ratio of IR700 molecule to the cetuximab is about 2.5:1, 2.6:1, 2.7:1, 2.8:1, 2.9:1, 3.0:1, 3.1:1, 3.2:1, 3.3:1 or 3.4:1.

106. The composition of any of embodiments 98-104, wherein the ratio of IR700 molecule to the cetuximab is between about 2.7:1 to about 3.2:1.

107. The composition of any of embodiments 98-106, wherein no more than at or about 15% of the cetuximab molecules in the composition are unconjugated with IR700.

108. The composition of any of embodiments 98-107, wherein less than at or about 10% of the cetuximab molecules in the composition are unconjugated with IR700.

109. The composition of any of embodiments 98-108, wherein the percentage of free dye in the composition is less than at or about 3%, less than at or about 2%, less than at or about 1%, or less than at or about 0.5%.

110. A composition comprising a population of cetuximab-IR700 conjugates, wherein a plurality of the conjugates in the composition each comprise IR700 conjugated to a cetuximab, at a lysine (K) in the light chain or the heavy chain of the cetuximab selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (1(5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab, and the composition comprises the features of:
(a) the ratio of IR700 molecules in the composition to the cetuximab molecules in the population is between about 2:1 and about 3:5,
(b) less than at or about 10% of the cetuximab molecules are unconjugated with IR700, and
(c) the percentage of free dye in the composition among all dye molecules in the composition is less than at or about 3%.

111. The composition of embodiment 110, wherein a plurality of the conjugates comprise a cetuximab conjugated with IR700 at K145 of the light chain.

112. The composition of embodiment 110 or 111, wherein a plurality of the conjugates comprise a cetuximab conjugated with IR700 at K215, K416 or K449 of the heavy chain.

113. The composition of any of embodiments 110-112, wherein a plurality of the conjugates comprise a cetuximab conjugated with at least three molecules of IR700.

114. The composition of any of embodiments 110-113, wherein the plurality comprises at least at or about 51%, at least at or about 55%, at least at or about 60%, at least at or about 70%, at least at or about 75% or at least at or about 80% of the conjugates in the composition.

115. The composition of any of embodiments 110-114, wherein the percentage of free dye in the composition is less than at or about 2%, less than at or about 1%, or less than at or about 0.5%.

116. The composition of any of embodiments 98-115, wherein the cetuximab comprises a heavy chain sequence set forth in SEQ ID NO: 1, a light chain sequence set forth in SEQ ID NO:2, or a combination thereof.

117. The composition of any of embodiments 98-116, wherein the percentage of free dye in the composition is substantially unchanged after storage for 6 months in dark or reduced light conditions.

118. The composition of any of embodiments 98-117, wherein the composition comprises at least at or about 95%, 96% 97% or 98% monomeric form of the conjugate.

119. The composition of any of embodiments 98-118, wherein the composition comprises less than at or about 5%, 4% or 3% high molecular weight species.

120. A method of killing a tumor or a cancer cell, the method comprising:
administering a pharmaceutical composition comprising the conjugate or the composition of any of embodiments 92-119 to a site at or proximal to the tumor or the cancer cell; and
irradiating an area proximal to the tumor or the cancer cell at a wavelength of about 600 nm to about 850 nm at a dose of from about 25 J cm⁻² to about 400 J cm⁻² or from about 25 J/cm of fiber length to about 500 J/cm of fiber length, thereby killing the tumor or the cancer cell.

121. A method of treating a disease or condition in a subject, the method comprising:
administering a pharmaceutical composition comprising the conjugate or the composition of any of embodiments 92-119 to a site at or proximal to the tumor or the cancer cell; and
irradiating an area proximal to a lesion or tumor in the subject at a wavelength of about 600 nm to about 850 nm at a dose of from about 25 J cm⁻² to about 400 J cm⁻² or from about 25 J/cm of fiber length to about 500 J/cm of fiber length, thereby treating the disease or condition.

122. The method of embodiment 120 or 121, wherein the irradiating step is carried out at a wavelength of 690±50 nm or at a wavelength of or about 690±20 nm.

123. The method of embodiment 122, wherein the irradiating step is carried out at a wavelength of about 690 nm.

124. The method of any of embodiments 121-123, wherein the disease or condition is a tumor or cancer.

125. The method of any of embodiments 120-124, wherein the tumor or the cancer cell comprises or the disease or condition is a tumor that is a carcinoma of the bladder, pancreas, colon, ovary, lung, breast, stomach, prostate, cervix, esophagus or head and neck.

126. The method of any of embodiments 120-124, wherein the tumor or the cancer cell comprises or the disease or condition is a cancer that is located at the head and neck, breast, liver, colon, ovary, prostate, pancreas, brain, cervix, bone, skin, eye, bladder, stomach, esophagus, peritoneum, or lung.

127. The method of embodiment 126, wherein the cancer is a head and neck cancer. 128. A method of manufacturing a stable conjugate, the method comprising:
a) contacting a cetuximab with an IR700 under conditions to produce a cetuximab-IR700 conjugate, wherein the conjugate comprises at least two lysine (K) positions conjugated to IR700 independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab;
b) subjecting the conjugate to a step during and/or subsequent to conjugation which substantially reduces IR700 non-specifically associated with the cetuximab; and
c) formulating the conjugate in a pharmaceutically acceptable buffer, wherein in each of steps a)-c), the only light to which the dye and conjugate are exposed has a wavelength within a range of about 400 nm to about 650 nm or has an intensity of less than at or about 500 lux.

129. The method of embodiment 128, wherein step b) comprises subjecting the conjugate to a glycine quenching reaction after completion of the conjugation reaction between IR700 and the cetuximab.

130. The method of embodiment 129, wherein the quenching reaction is performed overnight or for a duration of greater than at or about 6 hours.

131. A stable conjugate manufactured by the method of any of embodiments 128-130.

132. A composition comprising a plurality of conjugates, wherein the conjugates comprise IR700 conjugated to a cetuximab, wherein trypsin digestion of the composition produces a population of peptides comprising:
a) peptides of the heavy chain of cetuximab comprising an IR700 molecule conjugated to the lysine corresponding to position 215 (K215) of SEQ ID NO: 1;
b) peptides of the heavy chain of cetuximab comprising an IR700 molecule conjugated to the lysine corresponding to position 292 (K292) of SEQ ID NO: 1;
c) peptides of the heavy chain of cetuximab comprising an IR700 molecule conjugated to the lysine corresponding to position 416 (K416) of SEQ ID NO: 1; and
d) peptides of the light chain of cetuximab comprising an IR700 molecule conjugated to the lysine corresponding to position 145 (K145) of SEQ ID NO: 2.

133. The composition of embodiment 132, wherein the population of peptides further comprises:
e) peptides of the heavy chain of cetuximab comprising an IR700 molecule conjugated to the lysine corresponding to position 336 (K336) of SEQ ID NO: 1; and
f) peptides of the heavy chain of cetuximab comprising an IR700 molecule conjugated to the lysine corresponding to position 449 (K449) of SEQ ID NO: 1.

134. The composition of embodiment 132 or 133, wherein the population of peptides further comprises:
g) peptides of the light chain of cetuximab comprising an IR700 molecule conjugated to the lysine corresponding to position 107 (K107) of SEQ ID NO: 2;
h) peptides of the light chain of cetuximab comprising an IR700 molecule conjugated to the lysine corresponding to position 190 (K190) of SEQ ID NO: 2;
i) peptides of the heavy chain of cetuximab comprising an IR700 molecule conjugated to the lysine corresponding to position 5 (K5) of SEQ ID NO: 1; and
j) peptides of the heavy chain of cetuximab comprising an IR700 molecule conjugated to the lysine corresponding to position 75 (K75) of SEQ ID NO: 1.

135. The composition of any of embodiments 132-134, wherein the population of peptides further comprises one or more of:
k) peptides of the heavy chain of cetuximab comprising an IR700 molecule conjugated to the lysine corresponding to position 248 (K248) of SEQ ID NO: 1;
l) peptides of the heavy chain of cetuximab comprising an IR700 molecule conjugated to the lysine corresponding to position 328 (K328) of SEQ ID NO: 1;
m) peptides of the light chain of cetuximab comprising an IR700 molecule conjugated to the lysine corresponding to position 188 (K188) of SEQ ID NO: 2; and
n) peptides of the light chain of cetuximab comprising an IR700 molecule conjugated to the lysine corresponding to position 207 (K207) of SEQ ID NO: 2.

136. A composition comprising a plurality of conjugates, wherein the conjugates comprise IR700 conjugated to a cetuximab, wherein trypsin digestion of the composition produces a population of peptides comprising:
a) peptides of the heavy chain of cetuximab comprising an IR700 molecule conjugated to the lysine corresponding to position 5 (K5) of SEQ ID NO: 1;
b) peptides of the heavy chain of cetuximab comprising an IR700 molecule conjugated to the lysine corresponding to position 75 (K75) of SEQ ID NO: 1;
c) peptides of the heavy chain of cetuximab comprising an IR700 molecule conjugated to the lysine corresponding to position 215 (K215) of SEQ ID NO: 1;
d) peptides of the heavy chain of cetuximab comprising an IR700 molecule conjugated to the lysine corresponding to position 248 (K248) of SEQ ID NO: 1;
e) peptides of the heavy chain of cetuximab comprising an IR700 molecule conjugated to the lysine corresponding to position 292 (K292) of SEQ ID NO: 1;
f) peptides of the heavy chain of cetuximab comprising an IR700 molecule conjugated to the lysine corresponding to position 328 (K328) of SEQ ID NO: 1;
g) peptides of the heavy chain of cetuximab comprising an IR700 molecule conjugated to the lysine corresponding to position 336 (K336) of SEQ ID NO: 1;
h) peptides of the heavy chain of cetuximab comprising an IR700 molecule conjugated to the lysine corresponding to position 416 (K416) of SEQ ID NO: 1;
i) peptides of the heavy chain of cetuximab comprising an IR700 molecule conjugated to the lysine corresponding to position 449 (K449) of SEQ ID NO: 1;
j) peptides of the light chain of cetuximab comprising an IR700 molecule conjugated to the lysine corresponding to position 107 (K107) of SEQ ID NO: 2;
k) peptides of the light chain of cetuximab comprising an IR700 molecule conjugated to the lysine corresponding to position 145 (K145) of SEQ ID NO: 2;
l) peptides of the light chain of cetuximab comprising an IR700 molecule conjugated to the lysine corresponding to position 188 (K188) of SEQ ID NO: 2;
m) peptides of the light chain of cetuximab comprising an IR700 molecule conjugated to the lysine corresponding to position 190 (K190) of SEQ ID NO: 2; and
n) peptides of the light chain of cetuximab comprising an IR700 molecule conjugated to the lysine corresponding to position 207 (K207) of SEQ ID NO: 2.

137. The composition of any of embodiments 132-136, wherein the peptides are detected by positive ion mode mass spectrometry.

138. The composition of embodiment 137, wherein when extracted ion chromatograms (EIC) are generated for the peptides detected by the positive ion mode mass spectrometry:
the integrated area of the EIC peaks corresponding to peptides of a) is between at or about 3% and at or about 5% of the sum of the integrated area of the EIC peaks of the corresponding unmodified peptides and the integrated area of the EIC peaks corresponding the peptides of a);
the integrated area of the EIC peaks corresponding to peptides of b) is between at or about 3% and at or about 5% of the sum of the integrated area of the EIC peaks of the corresponding unmodified peptide and the integrated area of the EIC peaks corresponding the peptides of b);
the integrated area of the EIC peaks corresponding to peptides of c) is between at or about 8% and at or about 11% of the sum of the integrated area of the EIC peaks of the corresponding unmodified peptide and the integrated area of the EIC peaks corresponding the peptides of c);
the integrated area of the EIC peaks corresponding to peptides of d) is between at or about 0.5% and at or about 2.5% of the sum of the integrated area of the EIC peaks of the corresponding unmodified peptide and the integrated area of the EIC peaks corresponding the peptides of d);
the integrated area of the EIC peaks corresponding to peptides of e) is between at or about 8% and at or about 12% of the sum of the integrated area of the EIC peaks of the corresponding unmodified peptide and the integrated area of the EIC peaks corresponding the peptides of e);
the integrated area of the EIC peaks corresponding to peptides of f) is between at or about 0.2% and at or about 2.5% of the sum of the integrated area of the EIC peaks of the corresponding unmodified peptide and the integrated area of the EIC peaks corresponding the peptides of f);
the integrated area of the EIC peaks corresponding to peptides of g) is between at or about 4.5% and at or about 7% of the sum of the integrated area of the EIC peaks of the corresponding unmodified peptide and the integrated area of the EIC peaks corresponding the peptides of g);
the integrated area of the EIC peaks corresponding to peptides of h) is between at or about 9.5% and at or about 13% of the sum of the integrated area of the EIC peaks of the corresponding unmodified peptide and the integrated area of the EIC peaks corresponding the peptides of h);
the integrated area of the EIC peaks corresponding to peptides of i) is between at or about 6% and at or about 10% of the sum of the integrated area of the EIC peaks of the corresponding unmodified peptide and the integrated area of the EIC peaks corresponding the peptides of i);
the integrated area of the EIC peaks corresponding to peptides of j) is between at or about 2% and at or about 5% of the sum of the integrated area of the EIC peaks of the corresponding unmodified peptide and the integrated area of the EIC peaks corresponding the peptides of j);
the integrated area of the EIC peaks corresponding to peptides of k) is between at or about 7% and at or about 11% of the sum of the integrated area of the EIC peaks of the corresponding unmodified peptide and the integrated area of the EIC peaks corresponding the peptides of k);
the integrated area of the EIC peaks corresponding to peptides of l) is between at or about 0.5% and at or about 4% of the sum of the integrated area of the EIC peaks of the corresponding unmodified peptide and the integrated area of the EIC peaks corresponding the peptides of l);
the integrated area of the EIC peaks corresponding to peptides of m) is between at or about 1.5% and at or about 5% of the sum of the integrated area of the EIC peaks of the corresponding unmodified peptide and the integrated area of the EIC peaks corresponding the peptides of m); and
the integrated area of the EIC peaks corresponding to peptides of n) is between at or about 0.5% and at or about 4% of the sum of the integrated area of the EIC peaks of the corresponding unmodified peptide and the integrated area of the EIC peaks corresponding the peptides of n).

139. The composition of embodiment 138, wherein:
the integrated area of the EIC peaks corresponding to peptides of a) is about 3.8±1% of the sum of the integrated area of the EIC peaks of the corresponding unmodified peptides and the integrated area of the EIC peaks corresponding the peptides of a);
the integrated area of the EIC peaks corresponding to peptides of b) is about 3.5±1% of the sum of the integrated area of the EIC peaks of the corresponding unmodified peptides and the integrated area of the EIC peaks corresponding the peptides of b);
the integrated area of the EIC peaks corresponding to peptides of c) is about 10.0±1% of the sum of the integrated area of the EIC peaks of the corresponding unmodified peptides and the integrated area of the EIC peaks corresponding the peptides of c);
the integrated area of the EIC peaks corresponding to peptides of d) is about 1.7±1% of the sum of the integrated area of the EIC peaks of the corresponding unmodified peptides and the integrated area of the EIC peaks corresponding the peptides of d);
the integrated area of the EIC peaks corresponding to peptides of e) is about 10.2±1% of the sum of the integrated area of the EIC peaks of the corresponding unmodified peptides and the integrated area of the EIC peaks corresponding the peptides of e);
the integrated area of the EIC peaks corresponding to peptides of f) is about 1.3±1% of the sum of the integrated area of the EIC peaks of the corresponding unmodified peptides and the integrated area of the EIC peaks corresponding the peptides of f);
the integrated area of the EIC peaks corresponding to peptides of g) is about 5.9±1% of the sum of the integrated area of the EIC peaks of the corresponding unmodified peptides and the integrated area of the EIC peaks corresponding the peptides of g);
the integrated area of the EIC peaks corresponding to peptides of h) is about 11.2±1% of the sum of the integrated area of the EIC peaks of the corresponding unmodified peptides and the integrated area of the EIC peaks corresponding the peptides of h);
the integrated area of the EIC peaks corresponding to peptides of i) is about 7.6±1% of the sum of the integrated area of the EIC peaks of the corresponding unmodified peptides and the integrated area of the EIC peaks corresponding the peptides of i);
the integrated area of the EIC peaks corresponding to peptides of j) is about 3.4±1% of the sum of the integrated area of the EIC peaks of the corresponding unmodified peptides and the integrated area of the EIC peaks corresponding the peptides of j);
the integrated area of the EIC peaks corresponding to peptides of k) is about 9.3±1% of the sum of the integrated area of the EIC peaks of the corresponding unmodified peptides and the integrated area of the EIC peaks corresponding the peptides of k);
the integrated area of the EIC peaks corresponding to peptides of l) is about 2.1±1% of the sum of the integrated area of the EIC peaks of the corresponding unmodified peptides and the integrated area of the EIC peaks corresponding the peptides of l);
the integrated area of the EIC peaks corresponding to peptides of m) is about 3.5±1% of the sum of the integrated area of the EIC peaks of the corresponding unmodified peptides and the integrated area of the EIC peaks corresponding the peptides of m); and the integrated area of the EIC peaks corresponding to peptides of n) is about 2.0±1% of the sum of the integrated area of the EIC peaks of the corresponding unmodified peptides and the integrated area of the EIC peaks corresponding the peptides of n).

140. The composition of any of embodiments 132-139, wherein:
the amino acid sequence of the peptides of a) corresponds to amino acids 1-38 of SEQ ID NO: 1;
the amino acid sequence of the peptides of b) corresponds to amino acids 72-81 of SEQ ID NO: 1;
the amino acid sequence of the peptides of c) corresponds to amino acids 213-216 of SEQ ID NO: 1;
the amino acid sequence of the peptides of d) corresponds to amino acids 225-250 of SEQ ID NO: 1;
the amino acid sequence of the peptides of e) corresponds to amino acids 291-294 of SEQ ID NO: 1;
the amino acid sequence of the peptides of f) corresponds to amino acids 325-336 of SEQ ID NO: 1;
the amino acid sequence of the peptides of g) corresponds to amino acids 329-340 of SEQ ID NO: 1;
the amino acid sequence of the peptides of h) corresponds to amino acids 412-418 of SEQ ID NO: 1;
the amino acid sequence of the peptides of i) corresponds to amino acids 442-449 of SEQ ID NO: 1;
the amino acid sequence of the peptides of j) corresponds to amino acids 104-108 of SEQ ID NO: 2;
the amino acid sequence of the peptides of k) corresponds to amino acids 143-149 of SEQ ID NO: 2;
the amino acid sequence of the peptides of l) corresponds to amino acids 184-190 of SEQ ID NO: 2;
the amino acid sequence of the peptides of m) corresponds to amino acids 189-207 of SEQ ID NO: 2; and
the amino acid sequence of the peptides of n) corresponds to amino acids 191-211 of SEQ ID NO: 2.

141. A composition comprising a plurality of conjugates, wherein the conjugates comprise IR700 conjugated to cetuximab, and wherein trypsin digestion of the composition produces peptides that generate mass spectra comprising extracted ion chromatogram (EIC) peaks corresponding to:
peptides comprising an IR700 molecule conjugated to a lysine corresponding to position 215 (K215) of SEQ ID NO: 1, wherein the percent area of the conjugated EIC peak is at least at or about 9% of the total area of EIC peaks of the corresponding modified and unmodified polypeptide;
peptides comprising an IR700 molecule conjugated to a lysine corresponding to position 292 (K292) of SEQ ID NO: 1, wherein the percent area of the conjugated EIC peak is at least at or about 8% of the total area of EIC peaks of the corresponding modified and unmodified polypeptide;
peptides comprising an IR700 molecule conjugated to a lysine corresponding to position 416 (K416) of SEQ ID NO: 1, wherein the percent area of the conjugated EIC peak is at least at or about 8% of the total area of EIC peaks of the corresponding modified and unmodified polypeptide;
peptides comprising an IR700 molecule conjugated to a lysine corresponding to position 145 (K145) of SEQ ID NO: 2, wherein the percent area of the conjugated EIC peak is at least at or about 8% of the total area of EIC peaks of the corresponding modified and unmodified polypeptide.

142. The composition of embodiment of 141, wherein trypsin digestion of the composition further produces mass spectra comprising extracted ion chromatogram (EIC) peaks corresponding to one or more of:
peptides comprising an IR700 molecule conjugated to a lysine corresponding to position 449 (K449) of SEQ ID NO: 1, wherein the percent area of the conjugated EIC peak is at least at or about 5% of the total area of EIC peaks of the corresponding modified and unmodified polypeptide; and/or peptides comprising an IR700 molecule conjugated to a lysine corresponding to position 336 (K336) of SEQ ID NO: 1, wherein the percent area of the conjugated EIC peak is at least at or about 3.5% of the total area of EIC peaks of the corresponding modified and unmodified polypeptide.

143. The composition of embodiment 141 or 142, wherein trypsin digestion of the composition further produces mass spectra comprising extracted ion chromatogram (EIC) peaks corresponding to one or more of:
peptides comprising an IR700 molecule conjugated to a lysine corresponding to position 5 (K5) of SEQ ID NO: 1, wherein the percent area of the conjugated EIC peak is at least at or about 2% of the total area of EIC peaks of the corresponding modified and unmodified polypeptide;
peptides comprising an IR700 molecule conjugated to a lysine corresponding to position 75 (K75) of SEQ ID NO: 1, wherein the percent area of the conjugated EIC peak is at least at or about 2% of the total area of EIC peaks of the corresponding modified and unmodified polypeptide;
peptides comprising an IR700 molecule conjugated to a lysine corresponding to position 248 (K248) of SEQ ID NO: 1, wherein the percent area of the conjugated EIC peak is at least at or about 0.5% of the total area of EIC peaks of the corresponding modified and unmodified polypeptide;
peptides comprising an IR700 molecule conjugated to a lysine corresponding to position 328 (K328) of SEQ ID NO: 1, wherein the percent area of the conjugated EIC peak is at least at or about 0.5% of the total area of EIC peaks of the corresponding modified and unmodified polypeptide;
peptides comprising an IR700 molecule conjugated to a lysine corresponding to position 107 (K107) of SEQ ID NO: 2, wherein the percent area of the conjugated EIC peak is at least at or about 2% of the total area of EIC peaks of the corresponding modified and unmodified polypeptide;
peptides comprising an IR700 molecule conjugated to a lysine corresponding to position 188 (K188) of SEQ ID NO: 2, wherein the percent area of the conjugated EIC peak is at least at or about 0.5% of the total area of EIC peaks of the corresponding modified and unmodified polypeptide;
peptides comprising an IR700 molecule conjugated to a lysine corresponding to position 190 (K190) of SEQ ID NO: 2, wherein the percent area of the conjugated EIC peak is at least at or about 2% of the total area of EIC peaks of the corresponding modified and unmodified polypeptide; and/or
peptides comprising an IR700 molecule conjugated to a lysine corresponding to position 207 (K207) of SEQ ID NO: 2, wherein the percent area of the conjugated EIC peak is at least at or about 0.5% of the total area of EIC peaks of the corresponding modified and unmodified polypeptide.

144. A composition comprising a plurality of conjugates, wherein the conjugates comprise IR700 conjugated to cetuximab, and wherein trypsin digestion of the composition produces peptides that generate mass spectra comprising extracted ion chromatogram (EIC) peaks corresponding to:
peptides comprising an IR700 molecule conjugated to a lysine corresponding to position 5 (K5) of SEQ ID NO: 1, wherein the percent area of the conjugated EIC peak is at least at or about 2.5% of the total area of EIC peaks of the corresponding modified and unmodified polypeptide;
peptides comprising an IR700 molecule conjugated to a lysine corresponding to position 75 (K75) of SEQ ID NO: 1, wherein the percent area of the conjugated EIC peak is at least at or about 2.5% of the total area of EIC peaks of the corresponding modified and unmodified polypeptide;
peptides comprising an IR700 molecule conjugated to a lysine corresponding to position 215 (K215) of SEQ ID NO: 1, wherein the percent area of the conjugated EIC peak is at least at or about 9%, of the total area of EIC peaks of the corresponding modified and unmodified polypeptide;
peptides comprising an IR700 molecule conjugated to a lysine corresponding to position 248 (K248) of SEQ ID NO: 1, wherein the percent area of the conjugated EIC peak is at least at or about 0.5% of the total area of EIC peaks of the corresponding modified and unmodified polypeptide; and/or
peptides comprising an IR700 molecule conjugated to a lysine corresponding to position 292 (K292) of SEQ ID NO: 1, wherein the percent area of the conjugated EIC peak is about at least at or about 8.5%, of the total area of EIC peaks of the corresponding modified and unmodified polypeptide;
peptides comprising an IR700 molecule conjugated to a lysine corresponding to position 328 (K328) of SEQ ID NO: 1, wherein the percent area of the conjugated EIC peak is at least at or about 0.5% of the total area of EIC peaks of the corresponding modified and unmodified polypeptide;
peptides comprising an IR700 molecule conjugated to a lysine corresponding to position 336 (K336) of SEQ ID NO: 1, wherein the percent area of the conjugated EIC peak is at least at or about 4.5% of the total area of EIC peaks of the corresponding modified and unmodified polypeptide;
peptides comprising an IR700 molecule conjugated to a lysine corresponding to position 416 (K416) of SEQ ID NO: 1, wherein the percent area of the conjugated EIC peak is at least at or about 9%, of the total area of EIC peaks of the corresponding modified and unmodified polypeptide;
peptides comprising an IR700 molecule conjugated to a lysine corresponding to position 449 (K449) of SEQ ID NO: 1, wherein the percent area of the conjugated EIC peak is at least at or about 7% of the total area of EIC peaks of the corresponding modified and unmodified polypeptide;
peptides comprising an IR700 molecule conjugated to a lysine corresponding to position 107 (K107) of SEQ ID NO: 2, wherein the percent area of the conjugated EIC peak is at least at or about 2.5% of the total area of EIC peaks of the corresponding modified and unmodified polypeptide;
peptides comprising an IR700 molecule conjugated to a lysine corresponding to position 145 (K145) of SEQ ID NO: 1, wherein the percent area of the conjugated EIC peak is at least at or about 8.5%, of the total area of EIC peaks of the corresponding modified and unmodified polypeptide;
peptides comprising an IR700 molecule conjugated to a lysine corresponding to position 188 (K188) of SEQ ID NO: 2, wherein the percent area of the conjugated EIC peak is at least at or about 1% of the total area of EIC peaks of the corresponding modified and unmodified polypeptide;
peptides comprising an IR700 molecule conjugated to a lysine corresponding to position 190 (K190) of SEQ ID NO: 2, wherein the percent area of the conjugated EIC peak is at least at or about 2.5% of the total area of EIC peaks of the corresponding modified and unmodified polypeptide; and
peptides comprising an IR700 molecule conjugated to a lysine corresponding to position 207 (K207) of SEQ ID NO: 2, wherein the percent area of the conjugated EIC peak is at least at or about 1% of the total area of EIC peaks of the corresponding modified and unmodified polypeptide.

145. The composition of any of embodiments 141-144, wherein:
the percent area of the conjugated EIC peak is about 3.8±1% for the peptides comprising an IR700 molecule conjugated to a lysine corresponding to position 5 (K5) of SEQ ID NO: 1;
the percent area of the conjugated EIC peak is about 3.5±1% for the peptides comprising an IR700 molecule conjugated to a lysine corresponding to position 75 (1(75) of SEQ ID NO: 1;
the percent area of the conjugated EIC peak is about 10.0±1%, for the peptides comprising an IR700 molecule conjugated to a lysine corresponding to position 215 (K215) of SEQ ID NO: 1;
the percent area of the conjugated EIC peak is about 1.7±1% for the peptides comprising an IR700 molecule conjugated to a lysine corresponding to position 248 (K248) of SEQ ID NO: 1;
the percent area of the conjugated EIC peak is about 10.2±1% for the peptides comprising an IR700 molecule conjugated to a lysine corresponding to position 292 (K292) of SEQ ID NO: 1;
the percent area of the conjugated EIC peak is about 1.3±1% for the peptides comprising an IR700 molecule conjugated to a lysine corresponding to position 328 (K328) of SEQ ID NO: 1;
the percent area of the conjugated EIC peak about 5.9±1% for the peptides comprising an IR700 molecule conjugated to a lysine corresponding to position 336 (K336) of SEQ ID NO: 1;
the percent area of the conjugated EIC peak is about 11.2±1%, for the peptides comprising an IR700 molecule conjugated to a lysine corresponding to position 416 (K416) of SEQ ID NO: 1;
the percent area of the conjugated EIC peak about 7.6±1% for the peptides comprising an IR700 molecule conjugated to a lysine corresponding to position 449 (K449) of SEQ ID NO: 1;
the percent area of the conjugated EIC peak is about 3.4±1% for the peptides comprising an IR700 molecule conjugated to a lysine corresponding to position 107 (K107) of SEQ ID NO: 2;
the percent area of the conjugated EIC peak is about 9.3±1%, for the peptides comprising an IR700 molecule conjugated to a lysine corresponding to position 145 (K145) of SEQ ID NO: 2;
the percent area of the conjugated EIC peak is about 2.1±1% for the peptides comprising an IR700 molecule conjugated to a lysine corresponding to position 188 (K188) of SEQ ID NO: 2;
the percent area of the conjugated EIC peak is about 3.5±1% for the peptides comprising an IR700 molecule conjugated to a lysine corresponding to position 190 (K190) of SEQ ID NO: 2; and the percent area of the conjugated EIC peak is about 2±1% for the peptides comprising an IR700 molecule conjugated to a lysine corresponding to position 207 (K207) of SEQ ID NO: 2.

146. The composition of any of embodiments 141-145, wherein the peptides comprising an IR700 molecule conjugated to a lysine comprise one or more amino acid sequences selected from among:
the sequence of amino acids corresponding to amino acids 1-38, amino acids 72-81 amino acids 213-216, amino acids 225-250, amino acids 291-294, amino acids 325-336, amino acids 329-340, amino acids 412-418, and amino acids 442-449 of SEQ ID NO: 1, the sequence of amino acids corresponding to amino acids 104-108, amino acids 143-149, amino acids 184-190, amino acids 189-207, and amino acids 191-211 of SEQ ID NO: 2.

### VIII. EXAMPLES

The following examples are included for illustrative purposes only and are not intended to limit the scope of the invention.

### Example 1: Generation of Cetuximab-IR700 Conjugate

In this example, cetuximab-IR700 conjugates were generated using methods that limit the exposure of the dye and conjugate to light due to the photosensitivity of the dye, which included the use of low levels of green light having a wavelength from 425 to 575 nm and an intensity of less than 200 Lux in the manufacturing facility. The following buffers were used for conjugation: conjugation buffer (100 mM sodium phosphate, pH 8.65), quenching buffer (1.0 M glycine, pH 9) and final phosphate buffered saline (PBS) formulation buffer: (5.60 mM Na₂HPO₄, 1.058 mM KH₂PO₄, 154 mM NaCl, pH 7.1).

A. Preparation of Dye and Cetuximab

1. Cetuximab Preparation

Prior to conjugation, cetuximab was filtered through a 0.5/0.2 μm PES Millipore SHC filter, pooled, and stored at 2-8° C.

A concentration and buffer exchange step was then performed by ultrafiltration/diafiltration (UF/DF). The UF/DF device was cleaned and equilibrated with 100 mM sodium phosphate, pH 8.65 buffer. Prior to UF/DF operations, the pooled, filtered Cetuximab was warmed by placing it in an incubator at 25° C. for 120-150 min. The material was first concentrated to a target of 17-20 g/L and then diafiltered into PBS pH 7.1±0.2 buffer. The diafiltered Cetuximab product concentration was determined and then adjusted to a target concentration of 30-38 g/L. The resulting solution was filtered through a 0.5/0.2 μm PES Millipore SHC filter with a final concentration of 20-40 g/L. Prior to conjugation, the antibody solution was adjusted to a concentration of 10 g/L in 100 mM Sodium Phosphate pH 8.65±0.15 buffer, with a final target pH of 8.0-8.6.

2. Dye Preparation

Prior to conjugation, the IRDye 700DX NHS Ester (dye; Cat. No. 929-70011; Li-COR, Lincoln, Nebr.) was prepared by dissolving it to a concentration of 10 mg/mL in anhydrous DMSO. The steps were performed under green light (e.g., wavelength from 425 to 575 nm and an intensity of less than 200 Lux) to protect the dye from the wavelengths of light that are strongly absorbed by the dye.

B. Conjugation

The conjugation and quenching steps were performed in a vessel or tank containing diafiltered Cetuximab, wrapped in aluminum foil or similar for light protection. The steps were performed at room temperature under green light (e.g., wavelength from 425 to 575 nm and an intensity of less than 200 Lux) to protect the conjugate from photo-degradation.

The conjugation reaction was performed with IRDye 700DX NHS ester in DMSO, at a final molar ratio of 4:1 (IRDye 700DX NHS ester: cetuximab), to achieve incorporation of approximately 2-3 dye residues per cetuximab molecule. The IRDye 700DX NHS ester was added to the carboys containing cetuximab and mixed on a stir plate for 10-15 min. The conjugation reaction then proceeded for 60-90 min by placing the vessels in a 25° C. incubator (range 23-27° C.).

The conjugation reaction was quenched by mixing with 1.0 M glycine pH 9.0±0.2 to a final target concentration of 20 mM glycine. The vessels were incubated for an additional 16-24 hours at 25° C. (range 23-27° C.).

A final UF/DF step was performed to exchange the conjugated product into the final PBS formulation buffer. The quenched conjugate was transferred to the UF/DF system and was first concentrated to 9-11 g/L followed by diafiltration with 8-12 diavolumes of 10 mM Sodium Phosphate pH 7.1±0.2. The protein concentration was determined and if needed, further dilution with buffer was performed to reach a final target product concentration of 9-11 g/L.

A filtration through a 0.5/0.2 μm PES Millipore SHC filter was performed and the cetuximab-IR700 conjugate was stored in the dark at 2-8° C. in a vessel covered with aluminum foil to protect the contents from light. The steps were performed at room temperature under green light to protect the cetuximab-IR700 conjugate. The conjugate was diluted to a concentration of 6.7 g/L in 10 mM Sodium Phosphate pH 7.1±0.2. This conjugate solution was then diluted to a concentration of 5 g/L in 4-fold Concentrated Excipient Buffer: 10 mM Sodium Phosphate, 36% Trehalose, 0.06% PS-80 pH 7.1±0.2 (w/v), 0.5 to 0.8 mg/mL polysorbate-80. A final filtration step was performed using a 0.2 um PVDF Millipore Durapore filter and the resulting conjugate was formulated as 5 g/L in 10 mM Sodium Phosphate, 9% Trehalose, 0.02% polysorbate-80, pH 7.1±0.2.

The resulting conjugate was submitted for SEC-HPLC analysis to determine concentration, dye to antibody ratio (DAR), identity and purity, and to determine appearance, pH, bioburden, and endotoxin levels.

### Example 2: Mapping of the Conjugation Positions of the Cetuximab-IR700 Conjugate

A. IR700 Site Occupancy by Peptide Mapping

The cetuximab-IR700 conjugate was reduced using DTT in denaturing buffer (6M guanidine-HCl, 0.1M tris pH 8.0) followed by alkylation with iodoacetamide. Reduced and alkylated samples were desalted into trypsin digestion buffer (25 mM tris, 10 mM calcium chloride pH 7.5) and treated with protease at a ratio of 1:20 (protease:protein) and incubated at 37° C. overnight. To determine sites of IR700 conjugation, trypsin digested samples were treated with 1% TFA and heated at 37° C. for an additional two hours. TFA treated samples were analyzed by RP-HPLC analysis on a C18 column (Waters), with 220 nm and 690 nm detection, and MS detection, under conditions summarized in Table E1.

TABLE E1

| | Method | |
|---|---|---|
| | Parameter | Condition/Requirement |
| HPLC | Mobile | Mobile Phase A: 0.05% TFA in Water |
| Settings | Phase | Mobile Phase B: 0.04% TFA in Acetonitrile |
| | Flow Rate | 0.3 mL/min |
| | Column | XSelect CSH C18, 3.5 μm, 2.1 × 150 mm |
| | Detector | 220 nm, 690 nm |
| | Wavelength | |
| | Elution | Gradient |
| | Mode | |
| | Column | 50° C. |
| | Temperature | |
| Mass | Ion Polarity | Positive |
| Spectrometry | Gas | 325° C. |
| Settings | Temperature | |
| | Drying Gas | 9 L/min |
| | Nebulizer | 25 psig |
| | VCap | 3500 V |
| | Voltage | |
| | Fragmentor | 175 V |
| | Voltage | |
| | Collision | Slope—3.4 |
| | Energy | Offset—2.7 |

IR700 conjugation was analyzed at 690 nm and confirmed by mass spectrometry in positive ion mode. Quantitation of IR700 conjugation was determined by integrations of extracted ion chromatogram peaks correlating to conjugated peptides and unmodified peptides, using the formula below to determine conjugation levels:

$$Conjugation \% = \frac{Area of conjugated peptide}{Area of unmodified + conjugated peptide} * 100$$

Conjugation positions of IR700 dye on the light chain and heavy chain of cetuximab were mapped in three separately produced batches of the conjugate, and the results, retention times (RT), and masses (m/z) of the selected conjugated peptides and corresponding unmodified peptide(s) are shown Table E2 (light chain) and Table E3 (heavy chain).

TABLE E2

| Conjugation | RT | Conjugated | Unmodified | Conjugation % | | | |
|---|---|---|---|---|---|---|---|
| Site | (min) | Peptide (m/z) | Peptide (m/z) | Batch 1 | Batch 2 | Batch 3 | Avg |
| K49 | 58.5 | 1243.09 | 486.36, 633.82 | 0.5% | 0.4% | 0.5% | 0.5% |
| K107 | 54 | 714.34 | 502.32 | 3.7% | 3.0% | 3.4% | 3.4% |
| K126 | 50.2 | 1119.78 | 899.45, 973.52 | 0.4% | 0.5% | 0.4% | 0.4% |
| K145 | 53.5 | 829.36 | 347.19, 560.32 | 9.7% | 9.1% | 9.2% | 9.3% |
| K149 | 54.5 | 862.37 | 560.32, 1068.49 | 0.1% | 0.1% | 0.1% | 0.1% |
| K188 | 46 | 415.17 | 313.15, 625.28 | 1.7% | 2.3% | 2.3% | 2.1% |
| K190 | 43 | 970.76 | 938.47 | 3.5% | 3.2% | 3.7% | 3.5% |
| K207 | 50 | 1050.48 | 938.47, 523.26 | 2.0% | 1.9% | 2.1% | 2.0% |

TABLE E3

| Conjugation | | Conjugated | Native Peptide | Conjugation % | | | |
|---|---|---|---|---|---|---|---|
| Site | RT | peptide (m/z) | (m/z) | Batch 1 | Batch 2 | Batch 3 | Avg |
| K5 | 62 | 1224.84 | 598.38, 1188.26 | 3.7% | 3.8% | 3.9% | 3.8% |
| K43 | 65.8 | 1273.9 | 516.28, 1285.62 | 0.8% | 0.8% | 0.8% | 0.8% |
| K75 | 58.5 | 984.91 | 378.21, 462.21 | 3.3% | 3.7% | 3.5% | 3.5% |
| K81 | 64.5 | 1373.31 | 1333.85, 378.21 | 0.2% | 0.3% | 0.2% | 0.2% |
| K135 | 59 | 1086.84 | 593.83, 661.34 | 0.1% | 0.1% | 0.1% | 0.1% |
| K215 | 51 | 643.76 | 361.21, 1679.34 | 9.8% | 9.6% | 10.6% | 10.0% |
| K248 | 60 | 1205.22 | 711.87 | 1.7% | 1.7% | 1.6% | 1.7% |
| K250 | 57.5 | 1108.26 | 418.22, 711.87 | 0.2% | 0.2% | 0.1% | 0.2% |
| K276 | 60 | 914.2 | 839.4, 1070.02 | 0.4% | 0.4% | 0.4% | 0.4% |
| K290 | 52.5 | 892.71 | 839.4 | 0.4% | 0.7% | 0.6% | 0.6% |
| K292 | 49.5 | 424.16 | 501.31, 720.7 | 9.2% | 11.0% | 10.3% | 10.2% |
| K319 | 60 | 999.8 | 904.51 | 0.6% | 0.7% | 0.6% | 0.6% |
| K328 | 54 | 1018.47 | 419.76, 447.26 | 1.3% | 1.3% | 1.2% | 1.3% |
| K336 | 56 | 1018.98 | 419.76 | 5.9% | 6.1% | 5.8% | 5.9% |
| K362 | 57 | 850.37 | 319.15, 581.32 | 0.5% | 0.5% | 0.5% | 0.5% |
| K394 | 62 | 1288.31 | 937.46, 1272.57 | 0.2% | 0.4% | 0.3% | 0.3% |
| K416 | 52.5 | 529.89 | 575.34 | 10.2% | 12.3% | 11.1% | 11.2% |
| K449 | 61.5 | 779.33 | 394.73 | 7.4% | 8.1% | 7.2% | 7.6% |

B. Unconjugated Antibody by Strong-Anion Exchange (SAX) HPLC

Strong-anion exchange (SAX) HPLC was used to separate components in a sample based on charge. Conjugation between the cetuximab and the IRDye 700 DX (IR700) changed the charge of the antibody such that the conjugated and unconjugated antibody components have different charge profiles. For this reason, the SAX column was used to separate these two components from each other and to measure content of unconjugated antibody in the bulk drug substance and drug product. Content was reported as area percent relative to total peak area. Detection was by Absorbance at 280 nm with correction at Absorbance of 690 nm. This exemplary method was used for release and stability testing.

Samples were diluted with low conductivity buffer and applied onto a SAX HPLC column, under conditions shown in Table E4. The significant charge contributions from the IR700 dye provide resolution of the unconjugated cetuximab antibody to the IR700-conjugate by SAX-HPLC. As shown in Table E5, the percentage of unconjugated antibodies ranged from 4.6% to 7.1% in the various batches.

TABLE E4

| Method Parameter | Condition/Requirement |
|---|---|
| Mobile Phase | Equilibration: low conductivity, high pH |
| | Gradient: increasing sodium chloride concentration |
| | and reduction in pH |
| Flow Rate | 0.5 mL/min |
| Column | Thermo ProPac SAX 10, bioLC Analytical 4 mm × |
| | 250 mm |
| Detector | 280 nm^{a} |
| Wavelength | |
| Elution Mode | Gradient |
| Column | 30° C. |
| Temperature | |

TABLE E5

| Cetuximab-IR700 Conjugate Sample | % Unconjugated Antibody |
|---|---|
| Batch 1, 350 g scale | 6.9% |
| Batch 2, 350 g scale | 7.1% |
| Batch 3, 2 kg scale | 4.6% |

### Example 3: Assessment of Cetuximab-IR700 Conjugates

The cetuximab-IR700 conjugate batches were tested for EGFR binding and photoimmunotherapy (PIT) activities compared to a reference standard.

A. EGFR Binding

The binding of cetuximab-IR700 conjugate for human epidermal growth factor receptor (EGFR), relative to a reference standard, was measured by ELISA for each of the three batches of conjugates described in Example 2 above. The conjugates exhibited 103%, 97%, and 101% relative EGFR binding, respectively, compared to the reference standard.

B. Pit

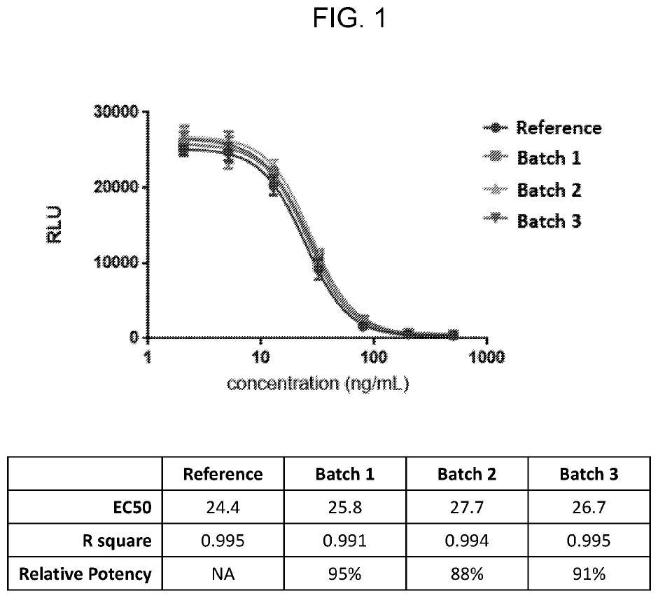

BxPC3 cells were plated in microtiter plate(s), leaving some wells cell-free (controls). The cells are allowed to adhere to the microtiter plate with overnight incubation at 37° C., 5% CO₂. Reference material and samples of the cetuximab-IR700 conjugate were serially diluted and applied to the cells in the wells of the plate. Complete cell culture media was added to the cell-free wells. After an hour of incubation at 37° C., 5% CO₂, the microtiter plates were irradiated with 690 nm light at a power density of 150 mW/cm², varying exposure time to achieve the desired light fluence (J/cm²). Following light exposure, the microtiter plate(s) containing the light treated cells was incubated at 37° C., 5% CO₂ for 22+/−2 hours. Cell viability was determined by the addition of a luminescent cell viability reagent, CellTiter-Glo 2.0, following overnight incubation. Results of the cell-based photoimmunotherapy (PIT) analysis for the different batches are shown in FIG. 1, and the relative potency of each of the batches is shown in Table E6 below. The raw luminescent units were collected and plotted to a 4-parameter logistic curve, demonstrating the cell-killing activity of the sample(s) relative to a reference standard.

TABLE E6

| Cetuximab-IR700 Conjugate Sample | % Relative Potency |
|---|---|
| Batch 1, 350 g scale | 95% |
| Batch 2, 350 g scale | 88% |
| Batch 3, 2 kg scale | 91% |

### Example 4: Residual Free IR700 Measurement by Size-Exclusion (SEC) HPLC of Cetuximab-IR700 Conjugate

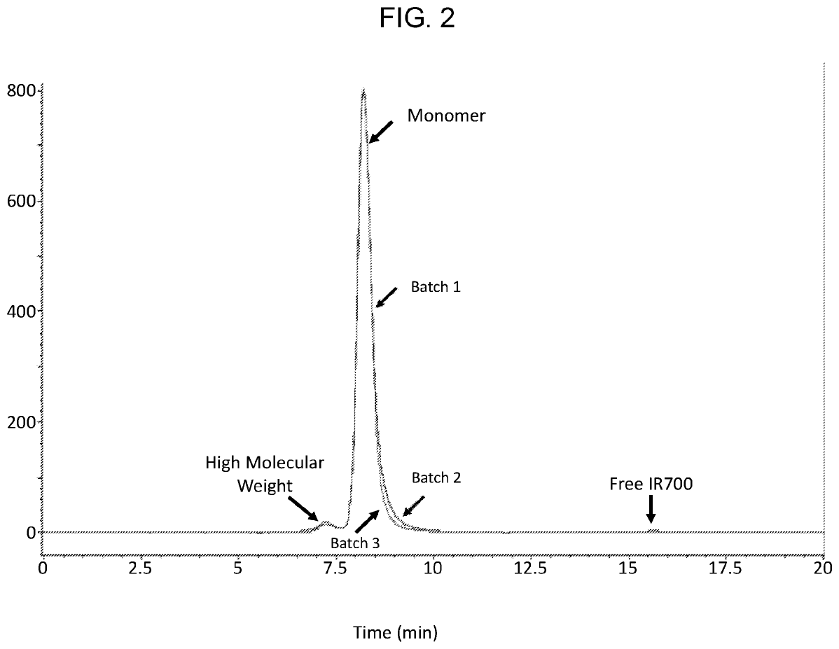

SEC-HPLC was employed to fractionate samples based on molecular weight and hydrodynamic ratio. The method was carried out under isocratic conditions. Detection of dye was by absorbance at 690 nm (A690) and 280 (A280) nm, and content was reported as area percent relative to total peak area. The use of the two wavelengths allowed for assessment of identity (at A690), product concentration (at A280 with background correction), purity and impurity content (at A690 and A280), free IR700 content (at A690) and dye-to-antibody ratio (DAR; A690 and A280). The correlates with stability-indicating measures, as increases in high molecular weight species and a decrease in main peak quantitation occur in samples exposed to light. This method was used to assess the 3 batches of conjugate (Batches 1-3) under conditions shown in Table E7, and results are shown in FIG. 2, where blue trace indicates batch No. 1, red trace indicates batch No. 2, and green trace indicates batch No. 3 of the cetuximab-IR700 conjugate. This method may be used for release and stability testing of bulk drug substance and drug product.

TABLE E7

| Method Parameter | Condition/Requirement |
|---|---|
| Mobile Phase | Phosphate buffered saline, pH 7.1 |
| Flow Rate | 0.5 mL/min |
| Column | Shodex Protein KW-803 |
| Nominal Column Load | 100 μg |
| Detector Wavelength | 280 nm and 690 nm |
| Run Time | 20 min |
| Elution Mode | Isocratic |
| Temperature | 25 ± 2° C. |
| Autosampler Temperature | 4 ± 2° C. |

As shown in Table E8 below, all 3 batches of the cetuximab-IR700 conjugate exhibited at least 97% monomer, less than 3% high molecular weight species (HMW), and less than or equal to 0.3% free IR700 dye (e.g., unconjugated dye).

TABLE E8

| | Cetuximab- | | | | | |
|---|---|---|---|---|---|---|
| | IR700 | | | | % | |
| Detection | Conjugate | % | % | % | Free | |
| Wavelength | Sample | Monomer | HMW | LMW | IR700 | DAR |
| 280 nm | Batch 1, | 98.4% | 1.6% | ND | NA | NA |
| | 350 g scale | | | | | |
| | Batch 2, | 98.3% | 1.7% | ND | NA | NA |
| | 350 g scale | | | | | |
| | Batch 3, | 97.8% | 2.2% | ND | NA | NA |
| | 2 kg scale | | | | | |
| 690 nm | Batch 1, | 98.0% | 2.0% | ND | 0.3% | 2.8 |
| | 350 g scale | | | | | |
| | Batch 2, | 97.9% | 2.1% | ND | 0.3% | 2.8 |
| | 350 g scale | | | | | |
| | Batch 3, | 97.0% | 3.0% | ND | 0.2% | 3.0 |
| | 2 kg scale | | | | | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A conjugate comprising at least two molecules of IR700 conjugated to at least two lysine (K) positions in a cetuximab, and wherein the at least two lysine positions are independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (1(207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab.

2. The conjugate of claim 1, comprising at least three molecules of IR700 conjugated to at least three lysine positions in the cetuximab.

3. The conjugate of claim 2, wherein the at least three lysine positions are independently selected from the group consisting of K107, K145, K188, K190, and K207 in the light chain and K5, K75, K215, K248, K292, K328, K336, K416, and K449 in the heavy chain.

4. The conjugate of any of claims 1-3, wherein at least one of the lysine positions conjugated to IR700 is selected from the group consisting of K145 in the light chain or K215, K416 or K449 in the heavy chain.

5. The conjugate of any of claims 1-4, wherein at least one molecule of IR700 is conjugated to a lysine in the light chain, and at least one molecule of IR700 is conjugated to a lysine in the heavy chain.

6. The conjugate of any of claims 1-5, wherein the conjugate is capable of being activated by light irradiation with a wavelength between 690 nm±50 nm and thereby exhibits a cell killing activity when the conjugate is bound to an epitope on the surface of the cell.

7. A composition comprising the conjugate of any of claims 1-6 and a pharmaceutically acceptable excipient.

8. A composition comprising a population of conjugates, wherein the conjugates in the population comprise IR700 conjugated to a cetuximab, wherein at least at or about 50%, 60%, 70%, 80%, 90%, or more than at or about 90% of the conjugates comprise at least two molecules of IR700 conjugated to at least two lysine (K) positions in the cetuximab, and wherein the two lysine positions are independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab.

9. The composition of claim 7 or 8, wherein at least at or about 50%, 60%, 70%, 80%, 90%, or more than at or about 90% of the conjugates comprise at least three molecules of IR700 conjugated to at least three lysine positions in the cetuximab.

10. The composition of claim 9, wherein the at least three lysine positions are independently selected from the group consisting of K107, K145, K188, K190, and K207 in the light chain and K5, K75, K215, K248, K292, K328, K336, K416, and K449 in the heavy chain.

11. The composition of any of claims 7-10, wherein at least at or about 50%, 60%, 70%, 80%, 90%, or more than at or about 90% of the conjugates comprise at least one molecule of IR700 conjugated to K145 in the light chain or K215, K416 or K449 in the heavy chain.

12. The composition of any of claims 7-11, wherein at least at or about 50%, 60%, 70%, 80%, 90%, or more than at or about 90% of the conjugates comprise a molecule of IR700 conjugated to K145 in the light chain and a molecule of IR700 conjugated to at least one of K215, K416 or K449 in the heavy chain.

13. The composition of any of claims 7-12, wherein the ratio of IR700 molecule to the cetuximab is between about 2:1 to about 4:1.

14. The composition of any of claims 7-13, wherein the ratio of IR700 molecule to the cetuximab is about 2.5:1, 2.6:1, 2.7:1, 2.8:1, 2.9:1, 3.0:1, 3.1:1, 3.2:1, 3.3:1 or 3.4:1.

15. The composition of any of claims 7-13, wherein the ratio of IR700 molecule to the cetuximab is between about 2.7:1 to about 3.2:1.

16. The composition of any of claims 7-15, wherein no more than at or about 15% of the cetuximab molecules in the composition are unconjugated with IR700.

17. The composition of any of claims 7-16, wherein less than at or about 10% of the cetuximab molecules in the composition are unconjugated with IR700.

18. The composition of any of claims 7-17, wherein the percentage of free dye in the composition is less than at or about 3%, less than at or about 2%, less than at or about 1%, or less than at or about 0.5%.

19. A composition comprising a population of cetuximab-IR700 conjugates, wherein a plurality of the conjugates in the composition each comprise IR700 conjugated to a cetuximab, at a lysine (K) in the light chain or the heavy chain of the cetuximab selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab, and the composition comprises the features of:
(a) the ratio of IR700 molecules in the composition to the cetuximab molecules in the population is between about 2:1 and about 3:5,
(b) less than at or about 10% of the cetuximab molecules are unconjugated with IR700, and
(c) the percentage of free dye in the composition among all dye molecules in the composition is less than at or about 3%.

20. The composition of claim 19, wherein a plurality of the conjugates comprise a cetuximab conjugated with IR700 at K145 of the light chain.

21. The composition of claim 19 or 20, wherein a plurality of the conjugates comprise a cetuximab conjugated with IR700 at K215, K416 or K449 of the heavy chain.

22. The composition of any of claims 19-21, wherein a plurality of the conjugates comprise a cetuximab conjugated with at least three molecules of IR700.

23. The composition of any of claims 19-22, wherein the plurality comprises at least at or about 51%, at least at or about 55%, at least at or about 60%, at least at or about 70%, at least at or about 75% or at least at or about 80% of the conjugates in the composition.

24. The composition of any of claims 19-23, wherein the percentage of free dye in the composition is less than at or about 2%, less than at or about 1%, or less than at or about 0.5%.

25. The composition of any of claims 7-24, wherein the cetuximab comprises a heavy chain sequence set forth in SEQ ID NO: 1, a light chain sequence set forth in SEQ ID NO:2, or a combination thereof.

26. The composition of any of claims 7-25, wherein the percentage of free dye in the composition is substantially unchanged after storage for 6 months in dark or reduced light conditions.

27. The composition of any of claims 7-26, wherein the composition comprises at least at or about 95%, 96% 97% or 98% monomeric form of the conjugate.

28. The composition of any of claims 7-27, wherein the composition comprises less than at or about 5%, 4% or 3% high molecular weight species.

29. A method of killing a tumor or a cancer cell, the method comprising:
administering a pharmaceutical composition comprising the conjugate or the composition of any of claims 1-28 to a site at or proximal to the tumor or the cancer cell; and
irradiating an area proximal to the tumor or the cancer cell at a wavelength of about 600 nm to about 850 nm at a dose of from about 25 J cm⁻² to about 400 J cm⁻² or from about 25 J/cm of fiber length to about 500 J/cm of fiber length, thereby killing the tumor or the cancer cell.

30. A method of treating a disease or condition in a subject, the method comprising:
administering a pharmaceutical composition comprising the conjugate or the composition of any of claims 1-28 to a site at or proximal to the tumor or the cancer cell; and
irradiating an area proximal to a lesion or tumor in the subject at a wavelength of about 600 nm to about 850 nm at a dose of from about 25 J cm⁻² to about 400 J cm⁻² or from about 25 J/cm of fiber length to about 500 J/cm of fiber length, thereby treating the disease or condition.

31. The method of claim 29 or 30, wherein the irradiating step is carried out at a wavelength of 690±50 nm or at a wavelength of or about 690±20 nm.

32. The method of claim 31, wherein the irradiating step is carried out at a wavelength of about 690 nm.

33. The method of any of claims 30-32, wherein the disease or condition is a tumor or cancer.

34. The method of any of claims 29-33, wherein the tumor or the cancer cell comprises or the disease or condition is a tumor that is a carcinoma of the bladder, pancreas, colon, ovary, lung, breast, stomach, prostate, cervix, esophagus or head and neck.

35. The method of any of claims 29-33, wherein the tumor or the cancer cell comprises or the disease or condition is a cancer that is located at the head and neck, breast, liver, colon, ovary, prostate, pancreas, brain, cervix, bone, skin, eye, bladder, stomach, esophagus, peritoneum, or lung.

36. The method of claim 35, wherein the cancer is a head and neck cancer.

37. A method of manufacturing a stable conjugate, the method comprising:
a) contacting a cetuximab with an IR700 under conditions to produce a cetuximab-IR700 conjugate, wherein the conjugate comprises at least two lysine (K) positions conjugated to IR700 independently selected from the group consisting of the lysine corresponding to position 107 (K107), the lysine corresponding to position 145 (K145), the lysine corresponding to position 188 (K188), the lysine corresponding to position 190 (K190), and the lysine corresponding to position 207 (K207) in the light chain of the cetuximab and the lysine corresponding to position 5 (K5), the lysine corresponding to position 75 (K75), the lysine corresponding to position 215 (K215), the lysine corresponding to position 248 (K248), the lysine corresponding to position 292 (K292), the lysine corresponding to position 238 (K328), the lysine corresponding to position 336 (K336), the lysine corresponding to position 416 (K416), and the lysine corresponding to position 449 (K449) in the heavy chain of the cetuximab;
b) subjecting the conjugate to a step during and/or subsequent to conjugation which substantially reduces IR700 non-specifically associated with the cetuximab; and
c) formulating the conjugate in a pharmaceutically acceptable buffer,
wherein in each of steps a)-c), the only light to which the dye and conjugate are exposed has a wavelength within a range of about 400 nm to about 650 nm or has an intensity of less than at or about 500 lux.

38. The method of claim 37, wherein step b) comprises subjecting the conjugate to a glycine quenching reaction after completion of the conjugation reaction between IR700 and the cetuximab.

39. The method of claim 38, wherein the quenching reaction is performed overnight or for a duration of greater than at or about 6 hours.

40. A stable conjugate manufactured by the method of any of claims 37-39.

41. A composition comprising a plurality of conjugates, wherein the conjugates comprise IR700 conjugated to a cetuximab, wherein trypsin digestion of the composition produces a population of peptides comprising:
a) peptides of the heavy chain of cetuximab comprising an IR700 molecule conjugated to the lysine corresponding to position 215 (K215) of SEQ ID NO: 1;
b) peptides of the heavy chain of cetuximab comprising an IR700 molecule conjugated to the lysine corresponding to position 292 (K292) of SEQ ID NO: 1;
c) peptides of the heavy chain of cetuximab comprising an IR700 molecule conjugated to the lysine corresponding to position 416 (K416) of SEQ ID NO: 1; and
d) peptides of the light chain of cetuximab comprising an IR700 molecule conjugated to the lysine corresponding to position 145 (K145) of SEQ ID NO: 2.

42. The composition of claim 41, wherein the population of peptides further comprises:
e) peptides of the heavy chain of cetuximab comprising an IR700 molecule conjugated to the lysine corresponding to position 336 (K336) of SEQ ID NO: 1; and
f) peptides of the heavy chain of cetuximab comprising an IR700 molecule conjugated to the lysine corresponding to position 449 (K449) of SEQ ID NO: 1.

43. The composition of claim 41 or 42, wherein the population of peptides further comprises:
g) peptides of the light chain of cetuximab comprising an IR700 molecule conjugated to the lysine corresponding to position 107 (K107) of SEQ ID NO: 2;
h) peptides of the light chain of cetuximab comprising an IR700 molecule conjugated to the lysine corresponding to position 190 (K190) of SEQ ID NO: 2;
i) peptides of the heavy chain of cetuximab comprising an IR700 molecule conjugated to the lysine corresponding to position 5 (K5) of SEQ ID NO: 1; and
j) peptides of the heavy chain of cetuximab comprising an IR700 molecule conjugated to the lysine corresponding to position 75 (K75) of SEQ ID NO: 1.

44. The composition of any of claims 41-43, wherein the population of peptides further comprises one or more of:
k) peptides of the heavy chain of cetuximab comprising an IR700 molecule conjugated to the lysine corresponding to position 248 (K248) of SEQ ID NO: 1;
l) peptides of the heavy chain of cetuximab comprising an IR700 molecule conjugated to the lysine corresponding to position 328 (K328) of SEQ ID NO: 1;
m) peptides of the light chain of cetuximab comprising an IR700 molecule conjugated to the lysine corresponding to position 188 (K188) of SEQ ID NO: 2; and
n) peptides of the light chain of cetuximab comprising an IR700 molecule conjugated to the lysine corresponding to position 207 (K207) of SEQ ID NO: 2.

45. A composition comprising a plurality of conjugates, wherein the conjugates comprise IR700 conjugated to a cetuximab, wherein trypsin digestion of the composition produces a population of peptides comprising:
a) peptides of the heavy chain of cetuximab comprising an IR700 molecule conjugated to the lysine corresponding to position 5 (K5) of SEQ ID NO: 1;
b) peptides of the heavy chain of cetuximab comprising an IR700 molecule conjugated to the lysine corresponding to position 75 (K75) of SEQ ID NO: 1;
c) peptides of the heavy chain of cetuximab comprising an IR700 molecule conjugated to the lysine corresponding to position 215 (K215) of SEQ ID NO: 1;
d) peptides of the heavy chain of cetuximab comprising an IR700 molecule conjugated to the lysine corresponding to position 248 (K248) of SEQ ID NO: 1;
e) peptides of the heavy chain of cetuximab comprising an IR700 molecule conjugated to the lysine corresponding to position 292 (K292) of SEQ ID NO: 1;
f) peptides of the heavy chain of cetuximab comprising an IR700 molecule conjugated to the lysine corresponding to position 328 (K328) of SEQ ID NO: 1;
g) peptides of the heavy chain of cetuximab comprising an IR700 molecule conjugated to the lysine corresponding to position 336 (K336) of SEQ ID NO: 1;
h) peptides of the heavy chain of cetuximab comprising an IR700 molecule conjugated to the lysine corresponding to position 416 (K416) of SEQ ID NO: 1;
i) peptides of the heavy chain of cetuximab comprising an IR700 molecule conjugated to the lysine corresponding to position 449 (K449) of SEQ ID NO: 1;
j) peptides of the light chain of cetuximab comprising an IR700 molecule conjugated to the lysine corresponding to position 107 (K107) of SEQ ID NO: 2;
k) peptides of the light chain of cetuximab comprising an IR700 molecule conjugated to the lysine corresponding to position 145 (K145) of SEQ ID NO: 2;
l) peptides of the light chain of cetuximab comprising an IR700 molecule conjugated to the lysine corresponding to position 188 (K188) of SEQ ID NO: 2;
m) peptides of the light chain of cetuximab comprising an IR700 molecule conjugated to the lysine corresponding to position 190 (K190) of SEQ ID NO: 2; and
n) peptides of the light chain of cetuximab comprising an IR700 molecule conjugated to the lysine corresponding to position 207 (K207) of SEQ ID NO: 2.

46. The composition of any of claims 41-45, wherein the peptides are detected by positive ion mode mass spectrometry.

47. The composition of claim 46, wherein when extracted ion chromatograms (EIC) are generated for the peptides detected by the positive ion mode mass spectrometry:
the integrated area of the EIC peaks corresponding to peptides of a) is between at or about 3% and at or about 5% of the sum of the integrated area of the EIC peaks of the corresponding unmodified peptides and the integrated area of the EIC peaks corresponding the peptides of a);
the integrated area of the EIC peaks corresponding to peptides of b) is between at or about 3% and at or about 5% of the sum of the integrated area of the EIC peaks of the corresponding unmodified peptide and the integrated area of the EIC peaks corresponding the peptides of b);
the integrated area of the EIC peaks corresponding to peptides of c) is between at or about 8% and at or about 11% of the sum of the integrated area of the EIC peaks of the corresponding unmodified peptide and the integrated area of the EIC peaks corresponding the peptides of c);
the integrated area of the EIC peaks corresponding to peptides of d) is between at or about 0.5% and at or about 2.5% of the sum of the integrated area of the EIC peaks of the corresponding unmodified peptide and the integrated area of the EIC peaks corresponding the peptides of d);
the integrated area of the EIC peaks corresponding to peptides of e) is between at or about 8% and at or about 12% of the sum of the integrated area of the EIC peaks of the corresponding unmodified peptide and the integrated area of the EIC peaks corresponding the peptides of e);
the integrated area of the EIC peaks corresponding to peptides of f) is between at or about 0.2% and at or about 2.5% of the sum of the integrated area of the EIC peaks of the corresponding unmodified peptide and the integrated area of the EIC peaks corresponding the peptides of f);
the integrated area of the EIC peaks corresponding to peptides of g) is between at or about 4.5% and at or about 7% of the sum of the integrated area of the EIC peaks of the corresponding unmodified peptide and the integrated area of the EIC peaks corresponding the peptides of g);
the integrated area of the EIC peaks corresponding to peptides of h) is between at or about 9.5% and at or about 13% of the sum of the integrated area of the EIC peaks of the corresponding unmodified peptide and the integrated area of the EIC peaks corresponding the peptides of h);
the integrated area of the EIC peaks corresponding to peptides of i) is between at or about 6% and at or about 10% of the sum of the integrated area of the EIC peaks of the corresponding unmodified peptide and the integrated area of the EIC peaks corresponding the peptides of i);
the integrated area of the EIC peaks corresponding to peptides of j) is between at or about 2% and at or about 5% of the sum of the integrated area of the EIC peaks of the corresponding unmodified peptide and the integrated area of the EIC peaks corresponding the peptides of j);
the integrated area of the EIC peaks corresponding to peptides of k) is between at or about 7% and at or about 11% of the sum of the integrated area of the EIC peaks of the corresponding unmodified peptide and the integrated area of the EIC peaks corresponding the peptides of k);
the integrated area of the EIC peaks corresponding to peptides of l) is between at or about 0.5% and at or about 4% of the sum of the integrated area of the EIC peaks of the corresponding unmodified peptide and the integrated area of the EIC peaks corresponding the peptides of l);
the integrated area of the EIC peaks corresponding to peptides of m) is between at or about 1.5% and at or about 5% of the sum of the integrated area of the EIC peaks of the corresponding unmodified peptide and the integrated area of the EIC peaks corresponding the peptides of m); and
the integrated area of the EIC peaks corresponding to peptides of n) is between at or about 0.5% and at or about 4% of the sum of the integrated area of the EIC peaks of the corresponding unmodified peptide and the integrated area of the EIC peaks corresponding the peptides of n).

48. The composition of claim 47, wherein:
the integrated area of the EIC peaks corresponding to peptides of a) is about 3.8±1% of the sum of the integrated area of the EIC peaks of the corresponding unmodified peptides and the integrated area of the EIC peaks corresponding the peptides of a);
the integrated area of the EIC peaks corresponding to peptides of b) is about 3.5±1% of the sum of the integrated area of the EIC peaks of the corresponding unmodified peptides and the integrated area of the EIC peaks corresponding the peptides of b);
the integrated area of the EIC peaks corresponding to peptides of c) is about 10.0±1% of the sum of the integrated area of the EIC peaks of the corresponding unmodified peptides and the integrated area of the EIC peaks corresponding the peptides of c);
the integrated area of the EIC peaks corresponding to peptides of d) is about 1.7±1% of the sum of the integrated area of the EIC peaks of the corresponding unmodified peptides and the integrated area of the EIC peaks corresponding the peptides of d);
the integrated area of the EIC peaks corresponding to peptides of e) is about 10.2±1% of the sum of the integrated area of the EIC peaks of the corresponding unmodified peptides and the integrated area of the EIC peaks corresponding the peptides of e);
the integrated area of the EIC peaks corresponding to peptides of f) is about 1.3±1% of the sum of the integrated area of the EIC peaks of the corresponding unmodified peptides and the integrated area of the EIC peaks corresponding the peptides of f);
the integrated area of the EIC peaks corresponding to peptides of g) is about 5.9±1% of the sum of the integrated area of the EIC peaks of the corresponding unmodified peptides and the integrated area of the EIC peaks corresponding the peptides of g);
the integrated area of the EIC peaks corresponding to peptides of h) is about 11.2±1% of the sum of the integrated area of the EIC peaks of the corresponding unmodified peptides and the integrated area of the EIC peaks corresponding the peptides of h);
the integrated area of the EIC peaks corresponding to peptides of i) is about 7.6±1% of the sum of the integrated area of the EIC peaks of the corresponding unmodified peptides and the integrated area of the EIC peaks corresponding the peptides of i);
the integrated area of the EIC peaks corresponding to peptides of j) is about 3.4±1% of the sum of the integrated area of the EIC peaks of the corresponding unmodified peptides and the integrated area of the EIC peaks corresponding the peptides of j);
the integrated area of the EIC peaks corresponding to peptides of k) is about 9.3±1% of the sum of the integrated area of the EIC peaks of the corresponding unmodified peptides and the integrated area of the EIC peaks corresponding the peptides of k);
the integrated area of the EIC peaks corresponding to peptides of l) is about 2.1±1% of the sum of the integrated area of the EIC peaks of the corresponding unmodified peptides and the integrated area of the EIC peaks corresponding the peptides of l);
the integrated area of the EIC peaks corresponding to peptides of m) is about 3.5±1% of the sum of the integrated area of the EIC peaks of the corresponding unmodified peptides and the integrated area of the EIC peaks corresponding the peptides of m); and
the integrated area of the EIC peaks corresponding to peptides of n) is about 2.0±1% of the sum of the integrated area of the EIC peaks of the corresponding unmodified peptides and the integrated area of the EIC peaks corresponding the peptides of n).

49. A composition comprising a plurality of conjugates, wherein the conjugates comprise IR700 conjugated to cetuximab, and wherein trypsin digestion of the composition produces peptides that generate mass spectra comprising extracted ion chromatogram (EIC) peaks corresponding to:
peptides comprising an IR700 molecule conjugated to a lysine corresponding to position 5 (K5) of SEQ ID NO: 1, wherein the percent area of the conjugated EIC peak is at least at or about 2.5% of the total area of EIC peaks of the corresponding modified and unmodified polypeptide;
peptides comprising an IR700 molecule conjugated to a lysine corresponding to position 75 (K75) of SEQ ID NO: 1, wherein the percent area of the conjugated EIC peak is at least at or about 2.5% of the total area of EIC peaks of the corresponding modified and unmodified polypeptide;
peptides comprising an IR700 molecule conjugated to a lysine corresponding to position 215 (K215) of SEQ ID NO: 1, wherein the percent area of the conjugated EIC peak is at least at or about 9%, of the total area of EIC peaks of the corresponding modified and unmodified polypeptide;
peptides comprising an IR700 molecule conjugated to a lysine corresponding to position 248 (K248) of SEQ ID NO: 1, wherein the percent area of the conjugated EIC peak is at least at or about 0.5% of the total area of EIC peaks of the corresponding modified and unmodified polypeptide; and/or
peptides comprising an IR700 molecule conjugated to a lysine corresponding to position 292 (K292) of SEQ ID NO: 1, wherein the percent area of the conjugated EIC peak is about at least at or about 8.5%, of the total area of EIC peaks of the corresponding modified and unmodified polypeptide;
peptides comprising an IR700 molecule conjugated to a lysine corresponding to position 328 (K328) of SEQ ID NO: 1, wherein the percent area of the conjugated EIC peak is at least at or about 0.5% of the total area of EIC peaks of the corresponding modified and unmodified polypeptide.
peptides comprising an IR700 molecule conjugated to a lysine corresponding to position 336 (K336) of SEQ ID NO: 1, wherein the percent area of the conjugated EIC peak is at least at or about 4.5% of the total area of EIC peaks of the corresponding modified and unmodified polypeptide.
peptides comprising an IR700 molecule conjugated to a lysine corresponding to position 416 (K416) of SEQ ID NO: 1, wherein the percent area of the conjugated EIC peak is at least at or about 9%, of the total area of EIC peaks of the corresponding modified and unmodified polypeptide;
peptides comprising an IR700 molecule conjugated to a lysine corresponding to position 449 (K449) of SEQ ID NO: 1, wherein the percent area of the conjugated EIC peak is at least at or about 7% of the total area of EIC peaks of the corresponding modified and unmodified polypeptide;
peptides comprising an IR700 molecule conjugated to a lysine corresponding to position 107 (K107) of SEQ ID NO: 2, wherein the percent area of the conjugated EIC peak is at least at or about 2.5% of the total area of EIC peaks of the corresponding modified and unmodified polypeptide;
peptides comprising an IR700 molecule conjugated to a lysine corresponding to position 145 (K145) of SEQ ID NO: 1, wherein the percent area of the conjugated EIC peak is at least at or about 8.5%, of the total area of EIC peaks of the corresponding modified and unmodified polypeptide;
peptides comprising an IR700 molecule conjugated to a lysine corresponding to position 188 (K188) of SEQ ID NO: 2, wherein the percent area of the conjugated EIC peak is at least at or about 1% of the total area of EIC peaks of the corresponding modified and unmodified polypeptide;
peptides comprising an IR700 molecule conjugated to a lysine corresponding to position 190 (K190) of SEQ ID NO: 2, wherein the percent area of the conjugated EIC peak is at least at or about 2.5% of the total area of EIC peaks of the corresponding modified and unmodified polypeptide; and
peptides comprising an IR700 molecule conjugated to a lysine corresponding to position 207 (K207) of SEQ ID NO: 2, wherein the percent area of the conjugated EIC peak is at least at or about 1% of the total area of EIC peaks of the corresponding modified and unmodified polypeptide.

50. The composition of claim 49, wherein:
the percent area of the conjugated EIC peak is about 3.8±1% for the peptides comprising an IR700 molecule conjugated to a lysine corresponding to position 5 (K5) of SEQ ID NO: 1;
the percent area of the conjugated EIC peak is about 3.5±1% for the peptides comprising an IR700 molecule conjugated to a lysine corresponding to position 75 (K75) of SEQ ID NO: 1;
the percent area of the conjugated EIC peak is about 10.0±1%, for the peptides comprising an IR700 molecule conjugated to a lysine corresponding to position 215 (K215) of SEQ ID NO: 1;
the percent area of the conjugated EIC peak is about 1.7±1% for the peptides comprising an IR700 molecule conjugated to a lysine corresponding to position 248 (K248) of SEQ ID NO: 1;
the percent area of the conjugated EIC peak is about 10.2±1% for the peptides comprising an IR700 molecule conjugated to a lysine corresponding to position 292 (K292) of SEQ ID NO: 1;
the percent area of the conjugated EIC peak is about 1.3±1% for the peptides comprising an IR700 molecule conjugated to a lysine corresponding to position 328 (K328) of SEQ ID NO: 1;
the percent area of the conjugated EIC peak about 5.9±1% for the peptides comprising an IR700 molecule conjugated to a lysine corresponding to position 336 (K336) of SEQ ID NO: 1;
the percent area of the conjugated EIC peak is about 11.2±1%, for the peptides comprising an IR700 molecule conjugated to a lysine corresponding to position 416 (K416) of SEQ ID NO: 1;
the percent area of the conjugated EIC peak about 7.6±1% for the peptides comprising an IR700 molecule conjugated to a lysine corresponding to position 449 (K449) of SEQ ID NO: 1;
the percent area of the conjugated EIC peak is about 3.4±1% for the peptides comprising an IR700 molecule conjugated to a lysine corresponding to position 107 (K107) of SEQ ID NO: 2;
the percent area of the conjugated EIC peak is about 9.3±1%, for the peptides comprising an IR700 molecule conjugated to a lysine corresponding to position 145 (K145) of SEQ ID NO: 2;
the percent area of the conjugated EIC peak is about 2.1±1% for the peptides comprising an IR700 molecule conjugated to a lysine corresponding to position 188 (K188) of SEQ ID NO: 2;
the percent area of the conjugated EIC peak is about 3.5±1% for the peptides comprising an IR700 molecule conjugated to a lysine corresponding to position 190 (K190) of SEQ ID NO: 2; and
the percent area of the conjugated EIC peak is about 2±1% for the peptides comprising an IR700 molecule conjugated to a lysine corresponding to position 207 (K207) of SEQ ID NO: 2.
